(19) United States
(12) Patent Application Publication
(10) Pub. No.: US 2006/0041111 A1
(43) Pub. Date: Feb. 23, 2006

(54) Dock 3 tumor suppressor gene

(76) Inventors: Vijay Yajnik, Chestnut Hill, MA (US); Charles Paulding, Weymouth, MA (US); Andrea McClatchey, Concord, MA (US); Daniel Haber, Chestnut Hill, MA (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/480,330

(22) PCT Filed: Jun. 10, 2002
(86) PCT No.: PCT/US02/18507

§ 371 Date: Nov. 12, 2004

(51) Int. Cl. C07K 14/52; A01K 67/027; C07H 21/02; C12P 21/02

(52) U.S. Cl. 536/23.1; 530/351; 435/69.5; 435/320.1; 435/354; 800/18

(57) ABSTRACT

The invention relates to a newly identified tumor suppressor gene, designated DOS (for Deleted in Osteosarcoma and alternatively referred to herein as DOCK 3) which has been cloned from human and mouse cells. The DOS nucleic acid and protein molecules and their use in the diagnosing and treating disorders characterized by aberrant DOS molecule expression are described.

### RELATED APPLICATIONS

This application claims priority under 35 U.S.C. § 119 to U.S. provisional application Ser. No. 60/297,382, filed Jun. 11, 2001.

### GOVERNMENT SUPPORT

This invention was made in part with government support under grant number R01CA58596 and 5T32DK07191-26 from the NIH. The government may have certain rights in this invention.

### FIELD OF THE INVENTION

This invention relates to a novel tumor suppressor gene that has been cloned from human and mouse tissue. The invention is directed to the isolated tumor suppressor nucleic acid, the proteins encoded by these nucleic acids, binding agents that selectively bind thereto, and various diagnostic, therapeutic and research uses of these compositions.

### BACKGROUND OF THE INVENTION

Cancer progression is caused by accumulation of multiple mutations that provide selective advantage during cancer growth, invasion and metastasis (1, 2, 3). While gain of function mutations occur in oncogenes, many of the genetic events that underlie cancer appear to be inactivating, or loss of function mutations affecting tumor suppressor genes (1). Tumor suppressor gene identified to date exhibit diverse cellular functions (4). Functional studies on these tumor suppressor genes have supported the original hypothesis that these genes represent potential bottlenecks in wide variety of cellular pathways (4). These include proliferation, differentiation, apoptosis and response to DNA damage. For example p53 and WT1 are DNA binding transcription factors; RB1, APC and possibly BRCA1 indirectly modulate transcription; P16 is an inhibitor of kinases required for cell cycle progression; PTEN is a novel phosphatase; NF2 is a cell structural component; VHL is a potential mediator of mRNA processing. A large number of genes are believed to be genomic caretakers and mutations in these genes cause microsatellite instability (MSH2, MLH1, PMS1 and PMS2) or chromosomal instability (p53, possibly BRCA1 and BRCA2). To date, genes involved in advanced stages of cancer progression such as invasion, angiogenesis and metastasis have not been identified (4, 5). They are likely to become evident over time with large-scale genome wide analysis.

The vast majority of cancers result from sporadic genetic events and only rare cases (less than 1%) have an inherited component (7, 8). However, the isolation of tumor suppressor genes has typically originated from genetic analysis of such rare inherited cancer syndromes (7). Linkage analysis on large families with cancer present in multiple generations allows identification of markers that co-segregate with cancer. In some cases cytogenetic abnormalities could also be observed either in sporadic or in germline tumors (7,9). For example, a small fraction of retinoblastomas have a homozygous deletion of RB1 gene (7). Rare Wilms tumor and colon cancer have deletions of WT1 and APC, respectively. These germline or sporadic homozygous deletions have been instrumental in tumor suppressor gene cloning efforts (7).

Allelic losses in tumors are typically detected as “loss of heterozygosity” or “LOH”. This represents loss of a polymorphic marker, commonly resulting from a large interstitial deletion or chromosomal non-disjunction event. While LOH is a common event in cancer, it only allows rough mapping of tumor suppressor loci (9). The large size of the LOH region (>10 Mb) makes the identification of the specific tumor suppressor gene targeted by mutation difficult. In contrast, homozygous deletions in tumors are typically small (<100 Kb) since they are restricted by the deletion of the flanking genes. Homozygous deletions occur by diverse mechanisms, including a small deletion in one allele accompanied by LOH of the second allele, or even large deletion of each allele whose common region of overlap is small. Identification of such homozygous deletions can be a powerful approach to identify tumor suppressor genes (7).

Significant technological advances have been made to identify regions of chromosomes involved in tumor progression. Analyses of metaphase chromosomes show chromosomal rearrangements in leukemia and lymphomas (10). This is more difficult in solid tumors where karyotyping is less commonly performed. Fluorescence in situ hybridization (FISH) has greatly improved the sensitivity and specificity of detecting chromosome aberrations (11, 12). However, its application in human malignancies is still limited because of complex karyotypes seen in clinical samples. Comparative genome hybridization (CGH) uses both normal and tumor genomes to identify regions in tumor DNA that have undergone changes in copy number (13). In this technique, normal and tumor DNA are labeled with two different haptens that fluoresce at different wavelengths. The probes are then hybridized to metaphase chromosomes in the presence of excess Cot-1 DNA thus inhibiting hybridization of labeled repetitive sequences. The ratio of the amount of two genomes that hybridize to specific areas of the chromosomes indicates the copy number of the two samples. CGH is currently limited to a resolution of 10 to 20 Mb and more sensitive in detecting amplifications rather than a small deletion (9). An alternative method, Representational Difference Analysis (RDA) is a PCR based subtractive hybridization technique, that is particularly applicable in isolating homozygous deletions in tumors (14, 18, 19). It has already been successful in isolating tumor suppressor genes PTEN and DMBT1 and has played a significant role in cloning of BRCA2 (15, 16, 17).

In view of the foregoing, a need exists to identify novel tumor suppressor genes to detect and treat various cancers. Preferably, such suppressor genes will have unique sequences that will permit the targeting of therapeutic agents for treating such cancers and the development of agents for detecting such cancers.

### SUMMARY OF THE INVENTION

The invention provides novel human and mouse tumor suppressor genes and is based, in part, on the discovery that this novel gene is deleted in a mouse osteosarcoma cell line. Using Representational Difference Analysis (RDA) on a mouse tumor model, we found a region of homozygous deletion in the mouse cell line. This region of deletion is homologous to human chromosome 7q31. We have cloned the gene residing in the deleted segment (DOS, for deleted in osteosarcoma, alternatively referred to herein as DOCK 3) from both mouse and humans. Human and mouse DOS protein sequence is about 97% identical. The protein has limited homology (approximately 30% identity) with three known genes, namely, DOCK180, myoblast city, and Ced 5. These three proteins are evolutionary conserved in both sequence and function and regulate actin cytoskeleton during cell migration. Accordingly, although not wishing to be bound to any particular theory or mechanism, we believe the DOS gene plays a role in regulating actin cytoskeleton in cell growth and cancer. Thus, the invention is directed to novel compositions of the DOS nucleic acids and proteins encoded thereby, as well as to agents that selectively bind to these novel molecules, and to diagnostic, therapeutic, and research applications of these compositions.

According to one aspect of the invention, an isolated nucleic acid molecule is provided. The isolated nucleic acid molecule is selected from the group consisting of:

(a) nucleic acid molecules which hybridize under stringent conditions to a nucleic acid molecule having a nucleotide sequence set forth as SEQ ID NO:1 or SEQ ID NO:3, and which code for a DOS protein,

(b) deletions, additions and substitutions of the nucleic acid molecules of (a),

(c) nucleic acid molecules that differ from the nucleic acid molecules of (a) or (b) in codon sequence due to the degeneracy of the genetic code, and

(d) complements of (a), (b) or (c).

The preferred isolated nucleic acids of the invention are DOS nucleic acid molecules which encode a DOS protein. As used herein, a DOS protein refers to a protein which is encoded by a nucleic acid having SEQ ID NO:1 or SEQ ID NO:3, or a functional fragment thereof, provided that the functional fragment encodes a protein which plays a role in tumor suppression, cytoskeletal organization, cell proliferation, cell migration, cellular growth and development, and/or cell-cell interaction.

In the preferred embodiments, the isolated nucleic acid molecule is SEQ ID NO:1 or SEQ ID NO:3

According to another aspect of the invention, further isolated nucleic acid molecules that are based on the above-noted DOS nucleic acid molecules are provided. In this aspect, the isolated nucleic acid molecules are selected from the group consisting of:

(a) a unique fragment of the nucleotide sequence set forth as SEQ ID NO:1 or set forth as SEQ ID NO:3 between 12 and 115 nucleotides in length or more and

(b) complements of (a),

wherein the unique fragments exclude nucleic acids having nucleotide sequences that are contained within SEQ ID NO:1 or SEQ ID NO:3, and that are known as of the filing date of the priority application.

In one embodiment of the invention an isolated unique nucleic acid fragment comprising SEQ ID NO: 31 of SEQ ID NO: 1 is provided.

In yet another aspect of the invention, Mutant DOS nucleic acid molecules are provided. The Mutant DOS nucleic acid molecules contain a sequence which is identical to SEQ ID NO:1 or SEQ ID NO:3, with the exception that the sequence includes one or more mutations, e.g., point mutations, deletion mutations, such that the Mutant DOS nucleic acid molecule does not encode a functional DOS protein. Rather, the Mutant DOS nucleic acid molecules encode a Mutant DOS protein, i.e., a protein which does not exhibit a DOS protein functional activity.

In preferred embodiments, the binding polypeptide is an antibody or antibody fragment, more preferably, an Fab or F(ab)₂ fragment of an antibody. Typically, the fragment includes a CDR3 region that is selective for the DOS protein or Mutant DOS protein. Any of the various types of antibodies can be used for this purpose, including monoclonal antibodies, humanized antibodies and chimeric antibodies.

According to a further aspect of the invention, pharmaceutical compositions containing the nucleic acids, proteins, and binding polypeptides of the invention are provided. The pharmaceutical compositions contain any of the foregoing therapeutic agents in a pharmaceutically acceptable carrier. Thus, in a related aspect, the invention provides a method for forming a medicament that involves placing a therapeutically effective amount of the therapeutic agent in the pharmaceutically acceptable carrier to form one or more doses.

According to another aspect of the invention, various diagnostic methods are provided. In general, the methods are for diagnosing “a disorder characterized by aberrant expression of a DOS molecule”. As used herein, “aberrant expression” refers to either or both of a decreased expression (including no expression) of the DOS molecule (nucleic acid or protein) or an increased expression of a “Mutant DOS molecule”. A Mutant DOS molecule refers to a DOS nucleic acid molecule which includes a mutation (point mutation, deletion) or to a DOS protein molecule (e.g., gene product of mutant DOS nucleic acid molecule) which includes a mutation, provided that the mutation results in a mutant DOS protein that does not have the DOS functional activity that is exhibited by a DOS protein as described herein. The diagnostic methods of the invention can be used to detect the presence of a disorder associated with aberrant expression of a DOS molecule, as well as to assess the progression and/or regression of the disorder such as in response to treatment (e.g., chemotherapy, radiation).

According to this aspect of the invention, the method for diagnosing a disorder characterized by aberrant expression of a DOS molecule involves: detecting in a first biological sample obtained from a subject, expression of a DOS molecule or a Mutant DOS molecule; wherein decreased expression of a DOS molecule or the increased expression of a Mutant DOS molecule compared to a control sample indicates that the subject has a disorder characterized by aberrant expression of a DOS molecule.

As used herein, a “disorder characterized by aberrant expression of a DOS molecule” refers to a disorder in which there is a detectable difference in the expression levels of DOS molecule(s) and/or Mutant DOS molecule(s) in selected cells of a subject compared to the

According to another aspect of the invention, an isolated nucleic acid molecule is provided. The isolated nucleic acid molecule is selected from the group consisting of:

(a) nucleic acid molecules which hybridize under stringent conditions to a nucleic acid molecule having a nucleotide sequence selected from the group consisting of SEQ ID NOs:5, 7, 9, 11, 13, 15, 17, 19, 21, 23, 25, 27, or 29,

(b) deletions, additions and substitutions of the nucleic acid molecules of (a),

(c) nucleic acid molecules that differ from the nucleic acid molecules of (a) or (b) in codon sequence due to the degeneracy of the genetic code, and

(d) complements of (a), (b) or (c)

According to yet another aspect of the invention, an expression vector comprising any of the isolated nucleic acid molecules of the invention operably linked to a promoter are provided. In a related aspect, host cells transformed or transfected with such expression vectors also are provided.

According to still a further aspect of the invention, a transgenic non-human animal comprising an expression vector of the invention is provided. Also provided is a transgenic non-human animal which has reduced expression of a DOS nucleic acid molecule or of a Mutant DOS nucleic acid molecule.

According to another aspect of the invention, an isolated polypeptide encoded by any of the foregoing isolated nucleic acid molecules of the invention is provided. Preferably, the isolated polypeptide comprises a polypeptide sequence selected from the group, consisting of SEQ ID NO: 2, 4, 9, 8, 10, 12, 14, 16, 18, 20, 22, 24, 26, 28, or 30.

In one aspect of the invention a functional protein fragment of SEQ ID NO: 2 comprising SEQ ID NO: 32 is provided.

In yet a further aspect of the invention, binding polypeptides that selectively bind to a DOS molecule and/or to a Mutant DOS molecule are provided. According to this aspect, the binding polypeptides bind to an isolated nucleic acid or protein of the invention, including binding to unique fragments thereof. Preferably, the binding polypeptides bind to a DOS protein, a Mutant DOS protein, or a unique fragment thereof. In certain particularly preferred embodiments, the binding polypeptide binds to a DOS protein but does not bind to a Mutant DOS protein, i.e., the binding polypeptides are selective for binding to the Mutant protein and can be used in various assays to detect the presence of the Mutant DOS protein without detecting DOS protein. control levels of these molecules. Thus, a disorder characterized by aberrant expression of a DOS molecule embraces underexpression (including no expression) of a DOS nucleic acid molecule or a DOS protein compared to control levels of these molecules, as well as overexpression of a Mutant DOS nucleic acid molecule or Mutant DOS protein compared to control levels of these molecules. Such differences in expression levels can be determined in accordance with the diagnostic methods of the invention as disclosed herein. Exemplary disorders that are characterized by aberrant expression of a DOS molecule include: various cancers and disorders associated with abnormal cytoskeleton organization, cell proliferation, cell migration, cellular growth and development, and/or cell-cell interaction.

In certain embodiments, the methods of the invention are to diagnose a cancer including, but not limited to, biliary tract cancer, brain cancer (including glioblastomas and medulloblastomas), breast cancer; cervical cancer; choriocarcinoma, colon cancer, endometrial cancer, esophageal cancer, gastric cancer, hematological neoplasms, including acute lymphocytic and myelogenous leukemia, multiple myeloma, AIDS associated leukemias and adult T-cell leukemia lymphoma, intraepithelial neoplasms, including Bowen's disease and Paget's disease, liver cancer, lung cancer, lymphomas, including Hodgkin's disease and lymphocytic lymphomas, neuroblastomas, oral cancer, including squamous cell carcinoma, ovarian cancer, including those arising from epithelial cells, stromal cells, germ cells and mesenchymal cells, pancreatic cancer, prostate cancer, rectal cancer, renal cancer including adenocarcinoma and Wilms tumor, sarcomas, including leiomyosarcoma, rhabdomyosarcoma, liposarcoma, fibrosarcoma and osteosarcoma, skin cancer, including melanoma, Kaposi's sarcoma, basocellular cancer and squamous cell cancer, testicular cancer, including germinal tumors (seminomas, and non-seminomas such as teratomas and choriocarcinomas), stromal tumors and germ cell tumors, and thyroid cancer, including thyroid adenocarcinoma and medullary carcinoma. In the preferred embodiments, the methods of the invention are useful for diagnosing Wilms tumor, ovarian carcinoma, renal cell carcinoma, osteosarcoma fibrosarcoma, prostate cnacer, colon cancer, and brain cancer.

In yet other embodiments, the diagnostic methods are useful for diagnosing the progression of a disorder. According to these embodiments, the methods further involve: detecting in a second biological sample obtained from the subject, expression of a DOS molecule or a Mutant DOS molecule, and comparing the expression of the DOS molecule or the Mutant DOS molecule in the first biological sample and the second biological sample. In these embodiments, a decrease in the expression of the DOS molecule in the second biological sample compared to the first biological sample or an increase in the expression of the Mutant DOS molecule in the second biological sample compared to the first biological sample indicates progression of the disorder.

In yet other embodiments, the diagnostic methods are useful for diagnosing the regression of a disorder. According to these embodiments, the methods further involve: detecting in a second biological sample obtained from the subject, expression of a DOS molecule or a Mutant DOS molecule, and comparing the expression of the DOS molecule or the Mutant DOS molecule in the first biological sample and the second biological sample. In these embodiments, an increase in the expression of the DOS molecule in the second biological sample compared to the first biological sample or a decrease in the expression of the Mutant DOS molecule in the second biological sample compared to the first biological sample indicates regression of the disorder.

In certain embodiments, the diagnostic methods of the invention detect a DOS molecule that is a DOS nucleic acid molecule or a Mutant DOS nucleic acid molecule as described above. In yet other embodiments, the methods involve detecting a DOS protein or Mutant DOS protein as described above.

Various detection methods can be used to practice the diagnostic methods of the invention. For example, when the methods can involve contacting the biological sample with an agent that selectively binds to the DOS molecule or to the Mutant DOS molecule to detect these molecules. In certain embodiments, the DOS molecule is a nucleic acid and the method involves using an agent that selectively binds to the DOS molecule or to the Mutant DOS molecule, e.g., a nucleic acid that hybridizes under stringent conditions to a nucleic acid molecule selected from the group consisting of SEQ ID NOs: 1, 3, 5, 7, 9, 11, 13, 15, 17, 19, 21, 23, 25, 27, and 29. In yet other embodiments, the DOS molecule is a protein and the method involves using an agent that selectively binds to the DOS molecule or to the Mutant DOS molecule, e.g., a binding polypeptide, such as an antibody, that selectively binds to a polypeptide selected from the group consisting of SEQ ID NOs: 2, 4, 6, 8, 10, 12, 14, 16, 18, 20, 22, 24, 26, 28, and 30.

In yet another embodiment an agent that selectively binds a fragment of a DOS molecule or to a fragment of a Mutant DOS molecule is provided. In certain embodiments the fragment of the DOS molecule or the fragment of the DOS molecule or the fragment of the Mutant DOS molecule is a nucleic acid molecule. One preferred nucleic acid fragment of the DOS molecule compressers SEQ ID NO: 31. In other embodiments, the fragment of the DOS molecule or the fragment of the Mutant DOS molecule is a polypeptide. One preferred polypeptide of the DOS molecule comprises SEQ ID NO: 32.

According to still another aspect of the invention, kits for performing the diagnostic methods of the invention are provided. The kits include nucleic acid-based kits or protein-based kits. According to the former embodiment, the kits include: one or more nucleic acid molecules that hybridize to a DOS nucleic acid molecule or to a Mutant DOS nucleic acid molecule under stringent conditions; one or more control agents; and instructions for the use of the nucleic acid molecules, and agents in the diagnosis of a DOS tumor. As used herein, A DOS tumor is disorder associated with aberrant expression of a DOS molecule. Nucleic acid-based kits optionally further include a first primer and a second primer, wherein the first primer and the second primer are constructed and arranged to selectively amplify at least a portion of an isolated DOS nucleic acid molecule selected from the group consisting of SEQ ID NOs: 1, 3, 5, 7, 9, 11, 13, 15, 17, 19, 21, 23, 25, 27, and 29. Alternatively, protein based-kits are provided. Such kits include: one or more binding polypeptides that selectively bind to a DOS protein or a Mutant DOS protein; one or more control agents; and instructions for the use of the binding polypeptides, and agents in the diagnosis of a disorder associated with aberrant expression of a DOS molecule. In the preferred embodiments, the binding polypeptides are antibodies or antigen-binding fragments thereof, such as those described above. In these and other embodiments, certain of the binding polypeptides bind to the Mutant DOS protein but do not bind to the DOS protein to further distinguish the expression of these proteins in a biological sample.

The invention also provides treatment methods. In general, the treatment methods involve administering an agent to increase expression of a DOS molecule and/or reduce expression of a Mutant DOS molecule. Thus, these methods include gene therapy applications. In certain embodiments, the method for treating a subject with a disorder characterized by aberrant expression of a DOS molecule, involves administering to the subject an effective amount of a DOS nucleic acid molecule to treat the disorder. In yet other embodiments, the method for treatment involves administering to the subject an effective amount of an anti-sense molecule to inhibit (reduce/eliminate) expression of a Mutant DOS nucleic acid molecule and, thereby, treat the disorder. An exemplary molecule for inhibiting expression of a Mutant DOS nucleic acid molecule is an anti-sense molecule that is selective for the mutant nucleic acid and that does not inhibit expression of the DOS nucleic acid molecule. Alternatively, the method for treating a subject with a disorder characterized by aberrant expression of a DOS molecule involves administering to the subject an effective amount of a DOS protein to treat the disorder. In yet another embodiment, the treatment method involves administering to the subject an effective amount of a binding polypeptide to inhibit a Mutant DOS protein and, thereby, treat the disorder. In certain preferred embodiments, the binding polypeptide is an antibody or an antigen-binding fragment thereof; more preferably, the antibodies or antigen-binding fragments are labeled with one or more cytotoxic agents

The invention provides various research methods and compositions. Thus, according to one aspect of the invention, a method for producing a DOS protein is provided. The method involves providing a DOS nucleic acid molecule operably linked to a promoter, wherein the DOS nucleic acid molecule encodes the DOS protein or a fragment thereof; expressing the DOS nucleic acid molecule in an expression system; and isolating the DOS protein or a fragment thereof from the expression system. Preferably, the DOS nucleic acid molecule has SEQ ID NO:1 or SEQ ID NO:3. According to yet another aspect of the invention, a method for producing a Mutant DOS protein is provided. This method involves: providing a Mutant DOS nucleic acid molecule operably linked to a promoter, wherein the Mutant DOS nucleic acid molecule encodes the Mutant DOS protein or a fragment thereof; expressing the Mutant DOS nucleic acid molecule in an expression system; and isolating the Mutant DOS protein or a fragment thereof from the expression system. Preferably, the Mutant DOS nucleic acid molecule has SEQ ID NO:1 or SEQ ID NO:3, with one or more point mutations or deletions to encode a Mutant DOS protein.

These and other aspects of the invention, as well as various advantages and utilities, will be more apparent with reference to the detailed description of the preferred embodiments.

### DETAILED DESCRIPTION OF THE INVENTION

The present invention in one aspect involves the cloning of a cDNA encoding a DOS protein. The sequence of the human gene is presented as SEQ ID NO:1, and the predicted amino acid sequence of this gene's protein product is presented as SEQ ID NO:2. The sequence of the mouse gene is presented as SEQ ID NO:3, and the predicted amino acid sequence of this gene's protein product is presented as SEQ ID NO:4. Sequence analysis shows that the human and mouse DOS proteins are about 97% identical. The invention thus involves in one aspect DOS proteins, genes encoding those proteins, functional modifications and variants of the foregoing, useful fragments of the foregoing, as well as therapeutic and diagnostic products and methods relating thereto.

According to one aspect of the invention, an isolated nucleic acid molecule is provided. The isolated nucleic acid molecule is selected from the group consisting of:

(a) nucleic acid molecules which hybridize under stringent conditions to a nucleic acid molecule having a nucleotide sequence set forth as SEQ ID NO:1 or SEQ ID NO:3, and which code for a DOS protein,

(b) deletions, additions and substitutions of the nucleic acid molecules of (a),

(c) nucleic acid molecules that differ from the nucleic acid molecules of (a) or (b) in codon sequence due to the degeneracy of the genetic code, and

(d) complements of (a), (b), or (c).

The preferred isolated nucleic acids of the invention are DOS nucleic acid molecules which encode a DOS protein. As used herein, a DOS protein refers to a protein which is encoded by a nucleic acid having SEQ ID NO:1 or SEQ ID NO:3, or a functional fragment thereof, or a functional equivalent thereof (e.g., a nucleic acid sequence encoding the same protein as encoded by SEQ ID NO:1 or SEQ ID NO:3), provided that the functional fragment or equivalent encodes a protein which exhibits a DOS functional activity. As used herein, a DOS functional activity refers to the ability of a DOS protein to modulate one or more of the following parameters: cytoskeletal organization, cell growth, cell proliferation, cell migration, and/or cell-cell interactions. An exemplary DOS functional activity is a tumor suppressor activity such as suppressing and/or reducing tumor cell growth, proliferation, and/or metastasis. Although not wishing to be bound to any particular theory or mechanism, it is believed that the DOS protein may affect at least some of the above-noted cell functions by interacting with actin and, thereby, modulating cytoskeletal organization.

In the preferred embodiments, the isolated nucleic acid molecule is SEQ ID NO:1 or SEQ ID NO:3.

The invention provides nucleic acid molecules which code for DOS proteins and which hybridize under stringent conditions to a nucleic acid molecule consisting of the nucleotide set forth in SEQ ID NO:1 or SEQ ID NO:3. Such nucleic acids may be DNA, RNA, composed of mixed deoxyribonucleotides and ribonucleotides, or may also incorporate synthetic non-natural nucleotides. Various methods for determining the expression of a nucleic acid and/or a polypeptide in normal and tumor cells are known to those of skill in the art and are described further below and in the Examples. As used herein, the term protein is meant to include large molecular weight proteins and peptides and low molecular weight peptides or fragments thereof.

The term “stringent conditions” as used herein refers to parameters with which the art is familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH2PO4 (pH 7), 0.5% SDS, 2 mM EDTA). SSC is 0.15M sodium chloride/0.15M sodium citrate, pH 7; SDS is sodium dodecyl sulphate; and EDTA is ethylenediaminetetracetic acid. After hybridization, the membrane upon which the DNA is transferred is washed at 2×SSC at room temperature and then at 0.1×SSC/0.1×SDS at temperatures up to 68° C.

The foregoing set of hybridization conditions is but one example of stringent hybridization conditions known to one of ordinary skill in the art. There are other conditions, reagents, and so forth which can be used, which result in a stringent hybridization. The skilled artisan will be familiar with such conditions, and thus they are not given here. It will be understood, however, that the skilled artisan will be able to manipulate the conditions in a manner to permit the clear identification of homologs and alleles of DOS nucleic acid molecules of the invention. The skilled artisan also is familiar with the methodology for screening cells and libraries for expression of such molecules which then are routinely isolated, followed by isolation of the pertinent nucleic acid molecule and sequencing.

In general homologs and alleles typically will share at least 40% nucleotide identity and/or at least 50% amino acid identity to SEQ ID NOs:1 or 3 and SEQ ID NOs:2 or 4, respectively, in some instances will share at least 50% nucleotide identity and/or at least 65% amino acid identity and in still other instances will share at least 60% nucleotide identity and/or at least 75% amino acid identity. Preferred homologs and alleles share nucleotide and amino acid identities with SEQ ID NO:1 or SEQ ID NO:3 and SEQ ID NO:2 or SEQ ID NO:4, respectively, and encode polypeptides of greater than 80%, more preferably greater than 90%, still more preferably greater than 95% and most preferably greater than 99% identity. The percent identity can be calculated using various, publicly available software tools developed by NCBI (Bethesda, Md.) that can be obtained through the internet (ftp:/ncbi.nlm.nih.gov/pub/). Exemplary tools include the BLAST system available at http://www.ncbi.nlm.nih.gov, which uses algorithms developed by Altschul et al. (Nucleic Acids Res. 25:3389-3402, 1997). Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acid molecules also are embraced by the invention.

In screening for DOS proteins, a Southern blot may be performed using the foregoing conditions, together with a radioactive probe. After washing the membrane to which the DNA is finally transferred, the membrane can be placed against X-ray film to detect the radioactive signal.

The invention also includes degenerate nucleic acid molecules which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating DOS protein. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

According to another aspect of the invention, further isolated nucleic acid molecules that are based on the above-noted DOS nucleic acid molecules are provided. In this aspect, the isolated nucleic acid molecules are selected from the group consisting of:

(a) a unique fragment of the nucleotide sequence set forth as SEQ ID NO:1, set as SEQ ID NO:3 between 12 and 32 nucleotides in length or more and

(b) complements of (a),

wherein the unique fragments exclude nucleic acids having nucleotide sequences that are contained within SEQ ID NO:1 or SEQ ID NO:3, and that are known as of the filing date of this application.

The invention also provides isolated unique fragments of SEQ ID NOs:1 or 3 or complements of SEQ ID NOs:1 or 3. In one embodiment, an isolated unique nucleic acid fragment of SEQ ID NO:1 comprising SEQ ID NO:31 is provided. A unique fragment is one that is a ‘signature’ for the larger nucleic acid. It, for example, is long enough to assure that its precise sequence is not found in molecules outside of the DOS nucleic acid molecules defined above. Those of ordinary skill in the art may apply no more than routine procedures to determine if a fragment is unique within the human or mouse genome. Unique fragments, however, exclude fragments completely composed of the nucleotide sequences that are contained within SEQ ID NO: 1 or SEQ ID NO: 3 and that are known as of the filing date of this application.

Unique fragments can be used as probes in Southern blot assays to identify such nucleic acid molecules, or can be used in amplification assays such as those employing PCR. As known to those skilled in the art, large probes such as 200 nucleotides or more are preferred for certain uses such as Southern blots, while smaller fragments will be preferred for uses such as PCR. Unique fragments also can be used to produce fusion proteins for generating antibodies or determining binding of the polypeptide fragments, or for generating immunoassay components. Likewise, unique fragments can be employed to produce nonfused fragments of the DOS polypeptides useful, for example, in the preparation of antibodies, in immunoassays. Unique fragments further can be used as antisense molecules to inhibit the expression of DOS nucleic acids and polypeptides, particularly for therapeutic purposes as described in greater detail below.

As will be recognized by those skilled in the art, the size of the unique fragment will depend upon its conservancy in the genetic code. Thus, some regions of SEQ ID NO:1 and/or SEQ ID NO:3 and its complement will require longer segments to be unique while others will require only short segments, typically between 12 and 32 nucleotides or more in length (e.g. 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42,43, 44, 45, 46, 47, 48, 49, 50, 51, 52, 53, 54, 55, 56, 57, 58, 59, 60, 61, 62, 63 ,64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115 or more), up to the entire length of the disclosed sequence. Many segments of the polynucleotide coding region or complements thereof that are 18 or more nucleotides in length will be unique. Those skilled in the art are well versed in methods for selecting such sequences, typically on the basis of the ability of the unique fragment to selectively distinguish the sequence of interest from non-DOS nucleic acid molecules. A comparison of the sequence of the fragment to those on known data bases typically is all that is necessary, although in vitro confirmatory hybridization and sequencing analysis may be performed.

A unique fragment can be a functional fragment. A functional fragment of a nucleic acid molecule of the invention is a fragment which retains some functional property of the larger nucleic acid molecule, such as coding for a functional polypeptide, binding to proteins, regulating transcription of operably linked nucleic acid molecules, and the like. One of ordinary skill in the art can readily determine using the assays described herein and those well known in the art to determine whether a fragment is a functional fragment of a nucleic acid molecule using no more than routine experimentation.

In yet another aspect of the invention, Mutant DOS nucleic acid molecules are provided. The Mutant DOS nucleic acid molecules contain a sequence which is identical to SEQ ID NO:1 or SEQ ID NO:3, with the exception that the sequence includes one or more mutations, e.g., deletions, additions or substitutions, such that the Mutant DOS nucleic acid molecule does not encode a functional DOS protein. Rather, the Mutant DOS nucleic acid molecules encode a Mutant DOS protein, i.e., a protein which does not exhibit a DOS protein functional activity.

According to another aspect of the invention, an isolated nucleic acid molecule is provided. The isolated nucleic acid molecule is selected from the group consisting of:

(a) nucleic acid molecules which hybridize under stringent conditions to a nucleic acid molecule having a nucleotide sequence selected from the group consisting of SEQ ID NOs:5, 7, 9, 11, 13, 15, 17, 19, 21, 23, 25, 27, or 29,

(b) deletions, additions and substitutions of the nucleic acid molecules of (a),

(c) nucleic acid molecules that differ from the nucleic acid molecules of (a) or (b) in codon sequence due to the degeneracy of the genetic code, and

(d) complements of (a), (b) or (c)

As used herein with respect to nucleic acid molecules, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid molecule is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid molecule may be substantially purified, but need not be. For example, a nucleic acid molecule that is isolated within a cloning or expression vector is not pure in that it may comprise only a tiny percentage of the material in the cell in which it resides. Such a nucleic acid molecule is isolated, however, as the term is used herein because it is readily manipulable by standard techniques known to those of ordinary skill in the art. An isolated nucleic acid molecule as used herein is not a naturally occurring chromosome.

As mentioned above, the invention embraces antisense oligonucleotides that selectively bind to a Mutant DOS nucleic acid molecule encoding a Mutant DOS protein. This is desirable in medical conditions wherein an aberrant DOS expression is not desirable, e.g., cancer. As used herein, a “Mutant DOS nucleic acid molecule” refers to a DOS nucleic acid molecule which includes a mutation (addition, deletion, or substitution) such that the Mutant DOS nucleic acid molecule does not encode a functional DOS protein. Rather, the Mutant DOS nucleic acid molecule encodes a Mutant DOS protein, i.e., a protein which does not exhibit a DOS protein functional activity. A “Mutant DOS protein” refers to a DOS protein that is a gene product of a mutant DOS nucleic acid molecule which includes a mutation that affects the functional activity of the DOS molecule. As used herein, the term “aberrant” refers to decreased expression (including zero or reduced expression) of the natural DOS molecule (nucleic acid or protein) or increased expression of a Mutant DOS molecule (nucleic acid or protein).

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind selectively with the target under physiological conditions, i.e., to hybridize substantially more to the target sequence than to any other sequence in the target cell under physiological conditions. Based upon SEQ ID NOs:1 or 3, or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nature Biotechnology 14: 840-844, 1996)

Most preferably, the antisense oligonucleotides comprise a complementary sequence of 20-30 bases. Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind. The present invention also provides for antisense oligonucleotides which are complementary to genomic DNA and/or cDNA corresponding to SEQ ID Nos: 1 and 3. Antisense to allelic or homologous cDNAs and genomic DNAs are enabled without undue experimentation.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus, modified oligonucleotides may include a 2′-O-alkylated ribose group. In addition, modified oligonucleotides may include sugars such as arabinose instead of ribose. The present invention, thus, contemplates pharmaceutical preparations containing modified antisense molecules that are complementary to and hybridizable with, under physiological conditions, nucleic acid molecules encoding DOS proteins, together with pharmaceutically acceptable carriers.

Antisense oligonucleotides may be administered as part of a pharmaceutical composition. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a patient. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

According to yet another aspect of the invention, an expression vector comprising any of the isolated nucleic acid molecules of the invention, preferably operably linked to a promoter is provided. In a related aspect, host cells transformed or transfected with such expression vectors also are provided.

Thus, it will also be recognized from the examples that the invention embraces the use of the DOS nucleic acid molecules in expression vectors, as well as to transfect host cells and cell lines, be these prokaryotic (e.g., E. coli, or eukaryotic (e.g., CHO cells, COS cells, yeast expression systems and recombinant baculovirus expression in insect cells). Especially useful are mammalian cells such as mouse, hamster, pig, goat, primate, etc. They can be of a wide variety of tissue types, including mast cells, fibroblasts, oocytes and lymphocytes, and they may be primary cells or cell lines. Specific examples include dendritic cells, U293 cells, peripheral blood leukocytes, bone marrow stem cells and embryonic stem cells. The expression vectors require that the pertinent sequence, i.e., those nucleic acids described supra, be operably linked to a promoter.

As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

It will also be recognized that the invention embraces the use of the DOS cDNA sequences or Mutant DOS cDNA sequences in expression vectors, as well as to transfect host cells and cell lines, be these prokaryotic (e.g., E. coli), or eukaryotic (e.g., CHO cells, COS cells, yeast expression systems and recombinant baculovirus expression in insect cells). Especially useful are mammalian cells such as human, mouse, hamster, pig, goat, primate, etc. They may be of a wide variety of tissue types, and include primary cells and cell lines. Specific examples include keratinocytes, peripheral blood leukocytes, bone marrow stem cells and embryonic stem cells. The expression vectors require that the pertinent-sequence, i.e., those nucleic acids described supra, be operably linked to a promoter.

According to still a further aspect of the invention, a transgenic non-human animal comprising an expression vector of the invention is provided, including a transgenic non-human animal which has reduced expression of a DOS nucleic acid molecule or a Mutant DOS nucleic acid molecule elevated expression of a DOS nucleic acid molecule or a Mutant DOS nucleic acid molecule.

As used herein, “transgenic non-human animals” includes non-human animals having one or more exogenous nucleic acid molecules incorporated in germ line cells and/or somatic cells. Thus the transgenic animal include “knockout” animals having a homozygous or heterozygous gene disruption by homologous recombination, animals having episomal or chromosomally incorporated expression vectors, etc. Knockout animals can be prepared by homologous recombination using embryonic stem cells as is well known in the art. The recombination can be facilitated by the cre/lox system or other recombinase systems known to one of ordinary skill in the art. In certain embodiments, the recombinase system itself is expressed conditionally, for example, in certain tissues or cell types, at certain embryonic or post-embryonic developmental stages, inducibly by the addition of a compound which increases or decreases expression, and the like. In general, the conditional expression vectors used in such systems use a variety of promoters which confer the desired gene expression pattern (e.g., temporal or spatial). Conditional promoters also can be operably linked to DOS nucleic acid molecules to increase or decrease expression of a DOS molecule in a regulated or conditional manner. Trans-acting negative or positive regulators of DOS activity or expression also can be operably linked to a conditional promoter as described above. Such trans-acting regulators include antisense DOS nucleic acid molecules, nucleic acid molecules which encode dominant negative DOS molecules, ribozyme molecules specific for DOS nucleic acid molecules, and the like. The transgenic non-human animals are useful in experiments directed toward testing biochemical or physiological effects of diagnostics or therapeutics for conditions characterized by increased or decreased DOS molecule expression. Other uses will be apparent to one of ordinary skill in the art. Thus, the invention also permits the construction of DOS gene “knock-outs” in cells and in animals, providing materials for studying certain aspects of cytoskeletal organization, cell migration, cancer, and metastasis.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding DOS protein, fragment, or variant thereof. The heterologous DNA (RNA) is placed under operable control of transcriptional elements to permit the expression of the heterologous DNA in the host cell.

Preferred systems for mRNA expression in mammalian cells are those such as pRc/CMV (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen, Carlsbad, Calif.), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.P1A recombinant is disclosed by Warnier et al., in intradermal injection in mice for immunization against P1A (Int. J. Cancer, 67:303-310, 1996).

The invention also embraces so-called expression kits, which allow the artisan to prepare a desired expression vector or vectors. Such expression kits include at least separate portions of each of the previously discussed coding sequences. Other components may be added, as desired, as long as the previously mentioned sequences, which are required, are included.

According to another aspect of the invention, an isolated protein encoded by any of the foregoing isolated nucleic acid molecules of the invention is provided. Preferably, the isolated protein comprises a protein selected from the group consisting of SEQ ID NO: 2, 4, 6, 8, 10, 12, 14, 16, 18, 20, 22, 24, 26, 28, and 30. The invention also embraces Mutant DOS proteins, such as those described in the Examples.

The invention also provides isolated proteins, which include the proteins of SEQ ID NOs:2, 4, 6, 8, 10, 12, 14, 16, 18, 20, 22, 24, 26, 28, and 30 and unique fragments of thereof. Such proteins are useful, for example, alone or as fusion proteins to generate antibodies, as a component(s) of an immunoassay or for determining the binding specificity of HLA molecules and/or CTL clones for DOS proteins.

As used herein, a DOS protein refers to a protein which is encoded by a nucleic acid having SEQ ID NO:1 or SEQ ID NO:3, a functional fragment thereof, or a functional equivalent thereof (e.g., a nucleic acid sequence encoding the same protein as encoded by SEQ ID NO:1 or SEQ ID NO:3), provided that the functional fragment or equivalent encodes a DOS protein which exhibits a DOS functional activity. As used herein, a DOS functional activity refers to the ability of a DOS protein to modulate one or more of the following parameters: cytoskeletal organization, cell growth, cell proliferation, cell migration, and/or cell-cell interactions. An exemplary DOS functional activity is a tumor suppressor activity such as suppressing and/or reducing tumor cell growth, proliferation, and/or metastasis. Although not wishing to be bound to any particular theory or mechanism, it is believed that the DOS protein may affect at least some of the above-noted cell functions by interacting with actin and, thereby, modulating cytoskeletal organization.

In one aspect of the invention a functional protein fragment of SEQ ID NO: 2 comprising SEQ ID NO: 32 is provided.

Proteins can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short polypeptides, including antigenic peptides (such as are presented by MHC molecules on the surface of a cell for immune recognition) also can be synthesized chemically using well-established methods of peptide synthesis.

Thus, as used herein with respect to proteins, “isolated” means separated from its native environment and present in sufficient quantity to permit its identification or use. Isolated, when referring to a protein or polypeptide, means, for example: (i) selectively produced by expression of a recombinant nucleic acid or (ii) purified as by chromatography or electrophoresis. Isolated proteins or polypeptides may, but need not be, substantially pure. The term “substantially pure” means that the proteins or polypeptides are essentially free of other substances with which they may be found in nature or in vivo systems to an extent practical and appropriate for their intended use. Substantially pure proteins may be produced by techniques well known in the art. Because an isolated protein may be admixed with a pharmaceutically acceptable carrier in a pharmaceutical preparation, the protein may comprise only a small percentage by weight of the preparation. The protein is nonetheless isolated in that it has been separated from the substances with which it may be associated in living systems, e.g. isolated from other proteins.

A fragment of a DOS protein, for example, generally has the features and characteristics of fragments including unique fragments as discussed above in connection with nucleic acid molecules. As will be recognized by those skilled in the art, the size of a fragment which is unique will depend upon factors such as whether the fragment constitutes a portion of a conserved protein domain. Thus, some regions of DOS proteins will require longer segments to be unique while others will require only short segments, typically between 5 and 12 amino acids (e.g. 5, 6, 7, 8, 9, 10, 11, and 12 amino acids long).

Unique fragments of a protein preferably are those fragments which retain a distinct functional capability of the protein. Functional capabilities which can be retained in a fragment of a protein include interaction with antibodies, interaction with other proteins or fragments thereof, selective binding of nucleic acid molecules, and enzymatic activity. One important activity is the ability to act as a signature for identifying the polypeptide. Another is the ability to provoke in a human an immune response to a Mutant DOS molecule but not provoke an immune response to a DOS molecule.

Those skilled in the art are well versed in methods for selecting unique amino acid sequences, typically on the basis of the ability of the fragment to selectively distinguish the sequence of interest from non-family members. A comparison of the sequence of the fragment to those on known data bases typically is all that is necessary.

The invention embraces variants of the DOS proteins described herein. As used herein, a “variant” of a DOS protein is a protein which contains one or more modifications to the primary amino acid sequence of a DOS protein. Modifications which create a DOS protein variant can be made to a DOS protein 1) to produce, increase, reduce, or eliminate—an activity of the DOS protein or Mutant DOS protein; 2) to enhance a property of the DOS protein, such as protein stability in an expression system or the stability of protein-protein binding; 3) to provide a novel activity or property to a DOS protein, such as addition of an antigenic epitope or addition of a detectable moiety; or 4) to provide equivalent or better binding to an HLA molecule. Modifications to a DOS protein or to a Mutant DOS protein are typically made to the nucleic acid molecule which encodes the protein, and can include deletions, point mutations, truncations, amino acid substitutions and additions of amino acids or non-amino acid moieties. Alternatively, modifications can be made directly to the protein, such as by cleavage, addition of a linker molecule, addition of a detectable moiety, such as biotin, addition of a fatty acid, and the like. Modifications also embrace fusion proteins comprising all or part of the DOS amino acid sequences. One of skill in the art will be familiar with methods for predicting the effect on protein conformation of a change in protein sequence, and can thus “design” a variant DOS polypeptide according to known methods. One example of such a method is described by Dahiyat and Mayo in Science 278:82-87, 1997, whereby proteins can be designed de novo. The method can be applied to a known protein to vary only a portion of the protein sequence. By applying the computational methods of Dahiyat and Mayo, specific variants of a DOS protein can be proposed and tested to determine whether the variant retains a desired conformation.

In general, variants include DOS proteins which are modified specifically to alter a feature of the protein unrelated to its desired physiological activity. For example, cysteine residues can be substituted or deleted to prevent unwanted disulfide linkages. Similarly, certain amino acids can be changed to enhance expression of a DOS protein by eliminating proteolysis by proteases in an expression system (e.g., dibasic amino acid residues in yeast expression systems in which KEX2 protease activity is present).

Mutations of a nucleic acid molecule which encode a DOS protein preferably preserve the amino acid reading frame of the coding sequence, and preferably do not create regions in the nucleic acid which are likely to hybridize to form secondary structures, such a hairpins or loops, which can be deleterious to expression of the variant protein.

Mutations can be made by selecting an amino acid substitution, or by random mutagenesis of a selected site in a nucleic acid which encodes the protein. Variant proteins are then expressed and tested for one or more activities to determine which mutation provides a variant protein with the desired properties. Further mutations can be made to variants (or to non-variant DOS proteins) which are silent as to the amino acid sequence of the protein, but which provide preferred codons for translation in a particular host. The preferred codons for translation of a nucleic acid in, e.g., E. coli, are well known to those of ordinary skill in the art. Still other mutations can be made to the noncoding sequences of a DOS gene or cDNA clone to enhance expression of the protein. The activity of variants of DOS proteins can be tested by cloning the gene encoding the variant DOS protein into a bacterial or mammalian expression vector, introducing the vector into an appropriate host cell, expressing the variant DOS protein, and testing for a functional capability of the DOS protein as disclosed herein. For example, the variant DOS protein can be tested for reaction with autologous or allogeneic sera. Preparation of other variant proteins may favor testing of other activities, as will be known to one of ordinary skill in the art.

The skilled artisan will also realize that conservative amino acid substitutions may be made in DOS proteins to provide functional variants of the foregoing proteins, i.e, the variants S which the functional capabilities of the DOS proteins. As used herein, a “conservative amino acid substitution” refers to an amino acid substitution which does not alter the relative charge or size characteristics of the protein in which the amino acid substitution is made. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

For example, upon determining that a peptide derived from a DOS protein plays a role in tumor suppression, cytoskeletal organization, cell proliferation, and/or cell migration, one can make conservative amino acid substitutions to the amino acid sequence of the peptide. The substituted peptides can then be tested for one or more of the above-noted functions, in vivo or in vitro. These variants can be tested for improved stability and are useful, inter alia, in pharmaceutical compositions.

Functional variants of DOS proteins, i.e., variants of proteins which retain the function of the DOS proteins, can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functional variants of the DOS proteins include conservative amino acid substitutions of proteins encoded by SEQ ID NOs:2 or 4. Conservative amino-acid substitutions in the amino acid sequence of DOS proteins to produce functional variants of DOS proteins typically are made by alteration of the nucleic acid molecule encoding a DOS protein (e.g. SEQ ID NO:1 or SEQ ID NO:3). Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a DOS protein. Where amino acid substitutions are made to a small unique fragment of a DOS protein the substitutions can be made by directly synthesizing the peptide. The activity of functional variants or fragments of DOS protein can be tested by cloning the gene encoding the altered DOS protein into a bacterial or mammalian expression vector, introducing the vector into an appropriate host cell, expressing the altered DOS protein, and testing for a functional capability of the DOS proteins as disclosed herein.

The invention as described herein has a number of uses, some of which are described elsewhere herein. First, the invention permits isolation of the DOS protein molecules. A variety of methodologies well-known to the skilled practitioner can be utilized to obtain isolated DOS molecules. The polypeptide may be purified from cells which naturally produce the protein by chromatographic means or immunological recognition. Alternatively, an expression vector may be introduced into cells to cause production of the protein. In another method, mRNA transcripts may be microinjected or otherwise introduced into cells to cause production of the encoded protein. Translation of mRNA in cell-free extracts such as the reticulocyte lysate system also may be used to produce polypeptide. Those skilled in the art also can readily follow known methods for isolating DOS proteins. These include, but are not limited to, immunochromatography, HPLC, size-exclusion chromatography, ion-exchange chromatography and immune-affinity chromatography.

The isolation and identification of DOS nucleic acid molecules also makes it possible for the artisan to diagnose a disorder characterized by aberrant expression of a DOS nucleic acid molecule or protein. These methods involve determining the aberrant expression of one or more DOS nucleic acid molecules and/or Mutant DOS nucleic acid molecules, and/or encoded DOS proteins and/or Mutant DOS proteins. In the former two situations, such determinations can be carried out via any standard nucleic acid determination assay, including the polymerase chain reaction, or assaying with labeled hybridization probes. In the latter two situations, such determinations can be carried out by screening patient antisera for recognition of the polypeptide or by assaying biological samples with binding partners (e.g., antibodies) for DOS proteins or Mutant DOS proteins.

The invention also provides, in certain embodiments, “dominant negative” polypeptides derived from DOS proteins. A dominant negative polypeptide is an inactive variant of a protein, which, by interacting with the cellular machinery, displaces an active protein from its interaction with the cellular machinery or competes with the active protein, thereby reducing the effect of the active protein. Dominant negative polypeptides are useful, or example, for preparing transgenic non-human animals to further characterize the functions of the DOS molecules and Mutant DOS molecules disclosed herein. For example, a dominant negative receptor which binds a ligand but does not transmit a signal in response to binding of the ligand can reduce the biological effect of expression of the ligand. Likewise, a dominant negative catalytically-inactive kinase which interacts normally with target proteins but does not phosphorylate the target proteins can reduce phosphorylation of the target proteins in response to a cellular signal. Similarly, a dominant negative transcription factor which binds to a promoter site in the control region of a gene but does not increase gene transcription can reduce the effect of a normal transcription factor by occupying promoter binding sites without increasing transcription.

The end result of the expression of a dominant negative polypeptide in a cell is a reduction in function of active proteins. One of ordinary skill in the art can assess the potential for a dominant negative variant of a protein, and using standard mutagenesis techniques to create one or more dominant negative variant polypeptides. For example, one of ordinary skill in the art can modify the sequence of DOS proteins by site-specific mutagenesis, scanning mutagenesis, partial gene deletion or truncation, and the like. See, e.g., U.S. Pat. No. 5,580,723 and Sambrook et al., Molecular Cloning: A Laboratory Manual, Second Edition, Cold Spring Harbor Laboratory Press, 1989. The skilled artisan then can test the population of mutagenized proteins for diminution in a selected and/or for retention of such an activity. Other similar methods for creating and testing dominant negative variants of a protein will be apparent to one of ordinary skill in the art.

In yet a further aspect of the invention, binding polypeptides that selectively bind to a DOS molecule and/or to a Mutant DOS molecule are provided. According to this aspect, the binding polypeptides bind to an isolated nucleic acid or protein of the invention, including binding to unique fragments thereof. Preferably, the binding polypeptides bind to a DOS protein, a Mutant DOS protein, or a unique fragment thereof. In certain particularly preferred embodiments, the binding polypeptide binds to a Mutant DOS protein but does not bind to a DOS protein, i.e., the binding polypeptides are selective for binding to the Mutant DOS protein and can be used in various assays to detect the presence of the Mutant DOS protein without detecting DOS protein. Such Mutant DOS protein binding polypeptides also can be used to selectively bind to a Mutant DOS molecule in a cell (in vivo or ex vivo) for imaging and therapeutic applications in which, for example, the binding polypeptide is tagged with a detectable label and/or a toxin for targetted delivery to the Mutant DOS molecule.

In preferred embodiments, the binding polypeptide is an antibody or antibody fragment, more preferably, an Fab or F(ab)₂ fragment of an antibody. Typically, the fragment includes a CDR3 region that is selective for the DOS protein or Mutant DOS protein. Any of the various types of antibodies can be used for this purpose, including monoclonal antibodies, humanized antibodies and chimeric antibodies.

Thus, the invention provides agents which bind to DOS proteins or Mutant DOS proteins encoded by DOS nucleic acid molecules or Mutant DOS nucleic acid molecules, respectively, and in certain embodiments preferably to unique fragments of the DOS proteins or Mutant DOS proteins. Such binding partners can be used in screening assays to detect the presence or absence of a DOS protein or a Mutant DOS protein and in purification protocols to isolate such DOS proteins. Likewise, such binding partners can be used to selectively target drugs, toxins or other molecules to cells which express Mutant DOS proteins. In this manner, cells present in solid or non-solid tumors which express Mutant DOS proteins can be treated with cytotoxic compounds. Such agents also can be used to inhibit the native activity of the DOS polypeptides, for example, by binding to such polypeptides, to further characterize the functions of these molecules

The invention, therefore, provides antibodies or fragments of antibodies having the ability to selectively bind to Mutant DOS proteins, and preferably to unique fragments thereof. Antibodies include polyclonal, monoclonal, and chimeric antibodies, prepared, e.g., according to conventional methodology.

The antibodies of the present invention thus are prepared by any of a variety of methods, including administering protein, fragments of protein, cells expressing the protein or fragments thereof and the like to an animal to induce polyclonal antibodies. The production of monoclonal antibodies is according to techniques well known in the art. As detailed herein, such antibodies may be used for example to identify tissues expressing protein or to purify protein. Antibodies also may be coupled to specific labeling agents for imaging or to antitumor agents, including, but not limited to, methotrexate, radioiodinated compounds, toxins such as ricin, other cytostatic or cytolytic drugs, and so forth.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of nonspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies. Thus, the invention involves polypeptides of numerous size and type that bind specifically to mutant DOS proteins. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptides and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent a completely degenerate or biased array. One then can select phage-bearing inserts which bind to a DOS protein or a Mutant DOS protein. This process can be repeated through several cycles of reselection of phage that bind to a DOS protein or a Mutant DOS protein. Repeated rounds lead to enrichment of phage bearing particular sequences. DNA sequence analysis can be conducted to identify the sequences of the expressed polypeptides. The minimal linear portion of the sequence that binds to the DOS protein or the Mutant DOS protein can be determined. One can repeat the procedure using a biased library containing inserts containing part or all of the minimal linear portion plus one or more additional degenerate residues upstream or downstream thereof. Thus, the DOS proteins of the invention can be used to screen peptide libraries, including phage display libraries, to identify and select peptide binding partners of the DOS proteins of the invention. Such molecules can be used, as described, for screening assays, for diagnostic assays, for purification protocols or for targeting drugs, toxins and/or labeling agents (e.g. radioisotopes, fluorescent molecules, etc.) to cells which express mutant DOS genes such as cancer cells which have aberrant DOS expression. Such binding agent molecules can also be prepared to bind complexes of an DOS protein and an HLA molecule by selecting the binding agent using such complexes.

As detailed herein, the foregoing antibodies and other binding molecules may be used for example to identify tissues expressing mutant protein or to purify mutant protein. Antibodies also may be coupled to specific diagnostic labeling agents for imaging of cells and tissues with aberrant DOS expression or to therapeutically useful agents according to standard coupling procedures. Diagnostic agents include, but are not limited to, barium sulfate, iocetamic acid, iopanoic acid, ipodate calcium, diatrizoate sodium, diatrizoate meglumine, metrizamide, tyropanoate sodium and radiodiagnostics including positron emitters such as fluorine-18 and carbon-11, gamma emitters such as iodine-123, technitium-99m, iodine-131 and indium-111, nuclides for nuclear magnetic resonance such as fluorine and gadolinium. Other diagnostic agents useful in the invention will be apparent to one of ordinary skill in the art. As used herein, “therapeutically useful agents” include any therapeutic molecule which desirably is targeted selectively to a cell or tissue selectively with an aberrant DOS expression, including antineoplastic agents, radioiodinated compounds, toxins, other cytostatic or cytolytic drugs, and so forth. Antineoplastic therapeutics are well known and include: aminoglutethimide, azathioprine, bleomycin sulfate, busulfan, carmustine, chlorambucil, cisplatin, cyclophosphamide, cyclosporine, cytarabidine, dacarbazine, dactinomycin, daunorubicin, doxorubicin, taxol, etoposide, fluorouracil, interferon, lomustine, mercaptopurine, methotrexate, mitotane, procarbazine HCl, thioguanine, vinblastine sulfate and vincristine sulfate. Additional antineoplastic agents include those disclosed in Chapter 52, Antineoplastic Agents (Paul Calabresi and Bruce A. Chabner), and the introduction thereto, pp. 1202-1263, of Goodman and Gilman's, The Pharmacological Basis of Therapeutics, Eighth Edition, 1990, McGraw-Hill, Inc. (Health Professions Division). Toxins can be proteins such as, for example, pokeweed anti-viral protein, cholera toxin, pertussis toxin, ricin, gelonin, abrin, diphtheria exotoxin, or Pseudomonas exotoxin. Toxin moieties can also be high energy-emitting radionuclides such as cobalt-60.

According to a further aspect of the invention, pharmaceutical compositions containing the nucleic acid molecules, proteins, and binding polypeptides of the invention are provided. The pharmaceutical compositions contain any of the foregoing therapeutic agents in a pharmaceutically acceptable carrier. Thus, in a related aspect, the invention provides a method for forming a medicament that involves placing a therapeutically effective amount of the therapeutic agent in the pharmaceutically acceptable carrier to form one or more doses.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents.

The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

The therapeutics of the invention can be administered by any conventional route, including injection or by gradual infusion over time. The administration may, for example, be oral, intravenous, intraperitoneal, intramuscular, intracavity, subcutaneous, or transdermal. When antibodies are used therapeutically, a preferred route of administration is by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712). Those of skill in the art can readily determine the various parameters and conditions for producing antibody aerosols without resort to undue experimentation. When using antisense preparations of the invention, slow intravenous administration is preferred.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The preparations of the invention are administered in effective amounts. An effective amount is that amount of a pharmaceutical preparation that alone, or together with further doses, stimulates the desired response. In the case of treating cancer, the desired response is inhibiting the progression of the cancer. This may involve only slowing the progression of the disease temporarily, although more preferably, it involves halting the progression of the disease permanently. In the case of stimulating an immune response, the desired response is an increase in antibodies or T lymphocytes which are specific for the immunogen(s) employed. These responses can be monitored by routine methods or can be monitored according to diagnostic methods of the invention discussed herein.

Where it is desired to stimulate an immune response using a therapeutic composition of the invention (e.g. a Mutant DOS protein fragment which is a unique fragment of the Mutant DOS molecule), this may involve the stimulation of a humoral antibody response resulting in an increase in antibody titer in serum, a clonal expansion of cytotoxic lymphocytes, or some other desirable immunologic response. It is believed that doses of immunogens ranging from one nanogram/kilogram to 100 milligrams/kilogram, depending upon the mode of administration, would be effective. The preferred range is believed to be between 500 nanograms and 500 micrograms per kilogram. The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

According to another aspect of the invention, various diagnostic methods are provided. In general, the methods are for diagnosing “a disorder characterized by aberrant expression of a DOS molecule”. As used herein, “aberrant expression” refers to either or both of a decreased expression (including no expression) of a DOS molecule (nucleic acid or protein) or an increased expression of a “Mutant DOS molecule”. A Mutant DOS molecule refers to a DOS nucleic acid molecule which includes a mutation (deletion, addition, or substitution) or to a DOS protein molecule (e.g., gene product of Mutant DOS nucleic acid molecule) which includes a mutation, provided that the mutation results in a Mutant DOS protein that does not have a DOS protein functional activity. The diagnostic methods of the invention can be used to detect the presence of a disorder associated with aberrant expression of a DOS molecule, as well as to assess the progression and/or regression of the disorder such as in response to treatment (e.g., chemotherapy, radiation).

According to this aspect of the invention, the method for diagnosing a disorder characterized by aberrant expression of a DOS molecule involves: detecting in a first biological sample obtained from a subject, expression of a DOS molecule or a Mutant DOS molecule; wherein decreased expression of a DOS molecule or the increased expression of a Mutant DOS molecule compared to a control sample indicates that the subject has a disorder characterized by aberrant expression of a DOS molecule.

As used herein, a “disorder characterized by aberrant expression of a DOS molecule” refers to a disorder in which there is a detectable difference in the expression levels of DOS molecule(s) and/or Mutant DOS molecule(s) in selected cells of a subject compared to the control levels of these molecules. Thus, a disorder characterized by aberrant expression of a DOS molecule embraces underexpression (including no expression) of a DOS nucleic acid molecule or a DOS protein compared to control levels of these molecules, as well as overexpression of a Mutant DOS nucleic acid molecule or Mutant DOS protein compared to control levels of these molecules. Such differences in expression levels can be determined in accordance with the diagnostic methods of the invention as disclosed herein. Exemplary disorders that are characterized by aberrant expression of a DOS molecule include: various cancers and disorders associated with abnormal cytoskeleton organization, cell proliferation, cell migration, cellular growth and development, and/or cell-cell interaction.

In certain embodiments, the methods of the invention are to diagnose a cancer including, but not limited to, biliary tract cancer, brain cancer (including glioblastomas and medulloblastomas), breast cancer; cervical cancer; choriocarcinoma, colon cancer, endometrial cancer, esophageal cancer, gastric cancer, hematological neoplasms, including acute lymphocytic and myelogenous leukemia, multiple myeloma, AIDS associated leukemias and adult T-cell lymphoma/leukemia, intraepithelial neoplasms, including Bowen's disease and Paget's disease, liver cancer, lung cancer, lymphomas, including Hodgkin's disease and lymphocytic lymphomas, neuroblastomas, oral cancer, including squamous cell carcinoma, ovarian cancer, including those arising from epithelial cells, stromal cells, germ cells and mesenchymal cells, pancreatic cancer, prostate cancer, rectal cancer, renal cancer including adenocarcinoma and Wilms tumor, sarcomas, including leiomyosarcoma, rhabdomyosarcoma, liposarcoma, fibrosarcoma and osteosarcoma, skin cancer, including melanoma, Kaposi's sarcoma, basocellular cancer and squamous cell cancer, testicular cancer, including germinal tumors (seminomas, and non-seminomas such as teratomas and choriocarcinomas), stromal tumors and germ cell tumors, and thyroid cancer, including thyroid adenocarcinoma and medullary carcinoma. In the preferred embodiments, the methods of the invention are useful for diagnosing Wilms tumor, ovarian carcinoma, renal cell carcinoma, osteosarcoma fibrosarcoma, prostate cancer, colon cancer and brain cancer.

In yet other embodiments, the diagnostic methods are useful for diagnosing the progression of a disorder. According to these embodiments, the methods further involve: detecting in a second biological sample obtained from the subject, expression of a DOS molecule or a Mutant DOS molecule, and comparing the expression of the DOS molecule or the Mutant DOS molecule in the first biological sample and the second biological sample. In these embodiments, a decrease in the expression of the DOS molecule in the second biological sample compared to the first biological sample or an increase in the expression of the Mutant DOS molecule in the second biological sample compared to the first biological sample indicates progression of the disorder.

In yet other embodiments, the diagnostic methods are useful for diagnosing the regression of a disorder. According to these embodiments, the methods further involve: detecting in a second biological sample obtained from the subject, expression of a DOS molecule or a Mutant DOS molecule, and comparing the expression of the DOS molecule or the Mutant DOS molecule in the first biological sample and the second biological sample. In these embodiments, an increase in the expression of the DOS molecule in the second biological sample compared to the first biological sample or a decrease in the expression of the Mutant DOS molecule in the second biological sample compared to the first biological sample indicates regression of the disorder.

In certain embodiments, the diagnostic methods of the invention detect a DOS molecule that is a DOS nucleic acid molecule or a Mutant DOS nucleic acid molecule as described above. In yet other embodiments, the methods involve detecting a DOS protein or Mutant DOS protein as described above.

Various detection methods can be used to practice the diagnostic methods of the invention. For example, when the methods can involve contacting the biological sample with an agent that selectively binds to the DOS molecule or to the Mutant DOS molecule to detect these molecules. In certain embodiments, the DOS molecule is a nucleic acid and the method involves using an agent that selectively binds to the DOS molecule or to the Mutant DOS molecule, e.g., a nucleic acid that hybridizes under stringent conditions to a nucleic acid sequence selected from the group consisiting of SEQ ID NOs: 1, 3, 5, 7, 9, 11, 13, 15, 17, 19, 21, 23, 25, 27, and 29. In yet other embodiments, the DOS molecule is a protein and the method involves using an agent that selectively binds to the DOS molecule or to the Mutant DOS molecule, e.g., a binding polypeptide, such as an antibody, that selectively binds to a protein sequence selected from the group consisting of SEQ ID NOs: 2, 4, 6, 8, 10, 12, 14, 16, 18, 20, 22, 24, 26, 28, and 30.

According to still another aspect of the invention, kits for performing the diagnostic methods of the invention are provided. The kits include nucleic acid-based kits or protein-based kits. According to the former embodiment, the kits include: one or more nucleic acid molecules that hybridize to a DOS nucleic acid molecule or to a Mutant DOS nucleic acid molecule under stringent conditions; one or more control agents; and instructions for the use of the nucleic acid molecules, and agents in the diagnosis of a disorder associated with aberrant expression of a DOS molecule. Nucleic acid-based kits optionally further include a first primer and a second primer, wherein the first primer and the second primer are constructed and arranged to selectively amplify at least a portion of an isolated DOS nucleic acid molecule selected from the group consisting of SEQ ID NOs: 1, 3, 5, 7, 9, 11, 13, 15, 17, 19, 21, 23, 25, 27, and 29. Alternatively, protein based-kits are provided. Such kits include: one or more binding polypeptides that selectively bind to a DOS protein or a Mutant DOS protein; one or more control agents; and instructions for the use of the binding polypeptides, and agents in the diagnosis of a disorder associated with aberrant expression of a DOS molecule. In the preferred embodiments, the binding polypeptides are antibodies or antigen-binding fragments thereof, such as those described above. In these and other embodiments, certain of the binding polypeptides bind to the Mutant DOS protein but do not bind to the DOS protein to further distinguish the expression of these proteins in a biological sample.

As used herein, a subject is a human, non-human primate, cow, horse, pig, sheep, goat, dog, cat or rodent. In all embodiments human and mouse DOS molecules and human subjects are preferred.

The biological sample can be located in vivo or in vitro. For example, the biological sample can be a tissue in vivo and the agent specific for the tumor associated nucleic acid molecule or polypeptide can be used to detect the presence of such molecules in the hematopoietic tissue (e.g., for imaging portions of the tissue that express the tumor associated gene products). Alternatively, the biological sample can be located in vitro (e.g., a blood sample, tumor biopsy, tissue extract). In a particularly preferred embodiment, the biological sample can be a cell-containing sample, more preferably a sample containing tumor cells. Samples of tissue and/or cells for use in the various methods described herein can be obtained through standard methods. Samples can be surgical samples of any type of tissue or body fluid. Samples can be used directly or processed to facilitate analysis (e.g., paraffin embedding). Exemplary samples include a cell, a cell scraping, a cell extract, a blood sample, a tissue biopsy, including punch biopsy, a tumor biopsy, a bodily fluid, a tissue, or a tissue extract or other methods.

The invention also provides treatment methods. As used herein, “treatment” includes preventing, delaying, abating or arresting the clinical symptoms of a disorder characterized by aberrant expression of a DOS molecule. Treatment also includes reducing or preventing tumor cell growth, proliferation, and/or metastasis.

In general, the treatment methods involve administering an agent to increase expression of a DOS molecule and/or reduce expression of a Mutant DOS molecule. Thus, these methods include gene therapy applications. In certain embodiments, the method for treating a subject with a disorder characterized by aberrant expression of a DOS molecule, involves administering to the subject an effective amount of a DOS nucleic acid molecule to treat the disorder. In yet other embodiments, the method for treatment involves administering to the subject an effective amount of an anti-sense molecule to inhibit (reduce/eliminate) expression of a Mutant DOS nucleic acid molecule and, thereby, treat the disorder. An exemplary molecule for inhibiting expression of a Mutant DOS nucleic acid molecule is an anti-sense molecule that is selective for the mutant nucleic acid and that does not inhibit expression of the DOS nucleic acid molecule. Alternatively, the method for treating a subject with a disorder characterized by aberrant expression of a DOS molecule involves administering to the subject an effective amount of a DOS protein to treat the disorder. In yet another embodiment, the treatment method involves administering to the subject an effective amount of a binding polypeptide to inhibit a Mutant DOS protein and, thereby, treat the disorder. In certain preferred embodiments, the binding polypeptide is an antibody or an antigen-binding fragment thereof; more preferably, the antibodies or antigen-binding fragments are labeled with one or more cytotoxic agents

The invention also contemplates gene therapy. The procedure for performing ex vivo gene therapy is outlined in U.S. Pat. No. 5,399,346 and in exhibits submitted in the file history of that patent, all of which are publicly available documents. In general, it involves introduction in vitro of a functional copy of a gene into a cell(s) of a subject which contains a defective copy of the gene, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO95/00654. In vivo gene therapy using vectors such as adenovirus, retroviruses, herpes virus, and targeted liposomes is also contemplated according to the invention.

In preferred embodiments, a virus vector for delivering a nucleic acid molecule encoding a DOS protein is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol 26:1951-1959, 1996). In preferred embodiments, the virus vector is an adenovirus.

Another preferred virus for certain applications is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In general, other preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Adenoviruses and retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., Gene Transfer and Expression, A Laboratory Manual, W. H. Freeman Co., New York (1990) and Murry, E. J. Ed. Methods in Molecular Biology, vol. 7, Humana Press, Inc., Cliffton, N.J. (1991).

Preferably the foregoing nucleic acid delivery vectors: (1) contain exogenous genetic material that can be transcribed and translated in a mammalian cell and that can suppress tumor cell growth and/or proliferation, and/or abnormal cytoskeletal organization, and/or abnormal cell growth, cell proliferation, cell migration, and/or cell-cell interaction in a host, and preferably (2) contain on a surface a ligand that selectively binds to a receptor on the surface of a target cell, such as a mammalian cell, and thereby gains entry to the target cell.

Various techniques may be employed for introducing nucleic acid molecules of the invention into cells, depending on whether the nucleic acid molecules are introduced in vitro or in vivo in a host. Such techniques include transfection of nucleic acid molecule-CaPO₄ precipitates, transfection of nucleic acid molecules associated with DEAE, transfection or infection with the foregoing viruses including the nucleic acid molecule of interest, liposome mediated transfection, and the like. For certain uses, it is preferred to target the nucleic acid molecule to particular cells. In such instances, a vehicle used for delivering a nucleic acid molecule of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid molecule delivery vehicle. Especially preferred are monoclonal antibodies. Where liposomes are employed to deliver the nucleic acid molecules of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acid molecules into cells, as is known by those skilled in the art. Such systems even permit oral delivery of nucleic acid molecules.

The invention provides various research methods and compositions. Thus, according to one aspect of the invention, a method for producing a DOS protein is provided. The method involves providing a DOS nucleic acid molecule operably linked to a promoter, wherein the DOS nucleic acid molecule encodes the DOS protein or a fragment thereof; expressing the DOS nucleic acid molecule in an expression system; and isolating the DOS protein or a fragment thereof from the expression system. Preferably, the DOS nucleic acid molecule has SEQ ID NO:1 or SEQ ID NO:3. According to yet another aspect of the invention, a method for producing a Mutant DOS protein is provided. This method involves: providing a Mutant DOS nucleic acid molecule operably linked to a promoter, wherein the Mutant DOS nucleic acid molecule encodes the Mutant DOS protein or a fragment thereof; expressing the Mutant DOS nucleic acid molecule in an expression system; and isolating the Mutant DOS protein or a fragment thereof from the expression system. Preferably, the Mutant DOS nucleic acid molecule has SEQ ID NO:1 or SEQ ID NO:3 with one or more deletions, additions, or substitutions to encode a Mutant DOS protein.

The invention further provides efficient methods of identifying pharmacological agents or lead compounds for agents which mimic the functional activity of a DOS molecule. Such DOS functional activities include tumor suppression, cytoskeletal organization, and cell migration. Generally, the screening methods involve assaying for compounds which modulate (up- or down-regulate) a DOS functional activity.

A wide variety of assays for pharmacological agents can be used in accordance with this aspect of the invention, including, labeled in vitro protein-protein binding assays, electrophoretic mobility shift assays, immunoassays, cell-based assays such as two- or three-hybrid screens, expression assays, etc. The assay mixture comprises a candidate pharmacological agent. Typically, a plurality of assay mixtures are run in parallel with different agent concentrations to obtain a different response to the various concentrations.

Typically, one of these concentrations serves as a negative control, i.e., at zero concentration of agent or at a concentration of agent below the limits of assay detection. Candidate agents encompass numerous chemical classes, although typically they are organic compounds. Preferably, the candidate pharmacological agents are small organic compounds, i.e., those having a molecular weight of more than 50 yet less than about 2500, preferably less than about 1000 and, more preferably, less than about 500. Candidate agents comprise functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid molecule, the agent typically is a DNA or RNA molecule, although modified nucleic acid molecules as defined herein are also contemplated.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease, inhibitors, nuclease inhibitors, antimicrobial agents, and the like may also be used.

An exemplary binding assay is described herein. In general the mixture of the foregoing assay materials is incubated under conditions whereby, but for the presence of the candidate pharmacological agent, the DOS molecule or the Mutant DOS molecule specifically binds the binding agent (e.g., antibody, complementary nucleic acid). The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours.

After incubation, the presence or absence of specific binding between the DOS molecule or the Mutant DOS molecule and one or more binding agents is detected by any convenient method available to the user. For cell free binding type assays, a separation step is often used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximum signal to noise ratios, primarily to minimize background binding, as well as for ease of separation and cost.

Separation may be effected for example, by removing a bead or dipstick from a reservoir, emptying or diluting a reservoir such as a microtiter plate well, rinsing a bead, particle, chromotograpic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific bindings such as salts, buffer, detergent, non-specific protein, etc. Where the solid substrate is a magnetic bead, the beads may be washed one or more times with a washing solution and isolated using a magnet.

Detection may be effected in any convenient way for cell-based assays such as two- or three-hybrid screens. For cell free binding assays, one of the components usually comprises, or is coupled to, a detectable label. A wide variety of labels can be used, such as those that provide direct detection (e.g., radioactivity, luminescence, optical or electron density, etc). or indirect detection (e.g., epitope tag such as the FLAG epitope, enzyme tag such as horseseradish peroxidase, etc.). The label may be bound to a DOS binding partner (e.g., polypeptide), or incorporated into the structure of the binding partner.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, strepavidin-biotin conjugates, etc. Methods for detecting the labels are well known in the art.

The invention will be more fully understood by reference to the following examples. These examples, however, are merely intended to illustrate the embodiments of the invention and are not to be construed to limit the scope of the invention.

### EXAMPLES

### Example 1

A. Introduction

The accumulation of somatic mutations in tumor cells provides selective advantage which results in cancer progression and metastasis. Characterization of these genetic events in cancer progression is essential for the understanding of tumor biology and identification of novel therapeutic targets. Representational difference analysis (RDA) is a powerful technique which has been successfully used to identify tumor suppressor genes by mapping homozygous deletions. However, RDA on human tumors is limited by common polymorphisms. Gene deletions occur in inbred mouse tumor models, which can be readily isolated by RDA because these strains have low prevalence of polymorphisms. Human orthologs of mouse tumor suppressor genes are then identified using gene prediction programs.

On the basis of this hypothesis a homozygous deletion was identified on mouse chromosome 12 in an osteosarcoma cell line derived from a NF2/p53 heterozygous mouse. This region is syntenic to a sequenced contig on human chromosome 7q31. With the assistance of gene prediction programs, DOS (Deleted in Osteosarcoma), a novel gene encoding a protein of 1966 amino acids was cloned. The protein has sequence homology to three known proteins in the database, namely DOCK180, myoblast city and Ced 5. These genes have been implicated in the regulation of actin cytoskeleton remodeling and integrin signaling. Since DOS appears to be deleted in mouse osteosarcoma, we believe DOS is a tumor suppressor gene and/or is involved in regulation of actin cytoskeleton reorganization and cell migration (e.g., as a negative regulator).

Sceening for Mutations in DOS in Mouse Cancer

We have available to us fifty cancer cell lines derived from p53 and NF2/p53 mouse tumor model to validate the involvement of DOS mutations in mouse tumorigenesis. These cell lines are screened for mutations by sequencing. We believe that DOS is frequently targeted by mutations in the p53 and NF2/p53 model systems and/or is mutated in other mouse tumor models, which can be confirmed by including tumors from other genetic backgrounds.

Screening for Mutations in DOS in Human Cancer

The DOS cDNA in twenty-five human cancer cell lines derived from a wide range of tumors is sequenced to determine if DOS is mutated in human cancer. The DOS in sporadic human cancer and matched normal specimens is also sequenced to search for loss of function mutations. Furthermore, whether DOS mutations occur early or late in carcinogenesis is investigated by corroborating mutations with histopathology.

Biochemical and Functional Characterization of DOS

DOS belongs to a family of proteins that affect morphogenesis and cell migration. To further characterize the role of DOS in such fundamental processes, epitope tagged, full length and C-terminus DOS mutant bacterial and mammalian expression constructs are prepared for functional studies. In particular, the osteosarcoma cell line with a deletion in DOS is useful in functional studies since it lacks the native protein. Antibodies to DOS are produced to perform immunoprecipitation and immunoblotting studies. Signal transduction studies focussing on the reorganization of actin cytoskeleton via Rho, Rac and Cdc42 are performed. Protein interaction studies are performed by GST pull down assays with SH3 domains of proteins such as Crk, CAS, Src and Nck. The role of DOS in signaling (e.g., integrin signaling), cell migration, proliferation, transformation and apoptosis is further characterized.

Strategies to Detect Allelic Loss in Tumors

Significant technological advances have been made to identify regions of chromosomes involved in tumor progression. Analyses of metaphase chromosomes show chromosomal rearrangements in leukemia and lymphomas (10). This is more difficult in solid tumors where karyotyping is less commonly performed. Fluorescence in situ hybridization (FISH) has greatly improved the sensitivity and specificity of detecting chromosome aberrations (11, 12). However, its application in human malignancies is still limited because of complex karyotypes seen in clinical samples. Comparative genome hybridization (CGH) uses both normal and tumor genomes to identify regions in tumor DNA that have undergone changes in copy number (13). In this technique, normal and tumor DNA are labeled with two different haptens that fluoresce at different wavelengths. The probes are then hybridized to metaphase chromosomes in the presence of excess Cot-1 DNA thus inhibiting hybridization of labeled repetitive sequences. The ratio of the amount of two genomes that hybridize to specific areas of the chromosomes indicates the copy number of the two samples. CGH is currently limited to a resolution of 10 to 20 Mb and more sensitive in detecting amplifications rather than a small deletion (9).

RDA as a Technology to Study Cancer Genetics

RDA, a PCR based subtractive hybridization technique, is particularly applicable in isolating homozygous deletions in tumors (14, 18, 19). It has already been successful in isolating tumor suppressor genes PTEN and DMBT1 and has played a significant role in cloning of BRCA2 (15, 16, 17). A detailed description of RDA follows.

In principle, a homozygous deletion in tumor DNA (also referred as target sequence in this proposal) should be readily identified by subtractive hybridization with normal DNA. Unfortunately, traditional subtractive hybridization techniques have had limited success because the mammalian genome is complex, not allowing sufficient time for single copy sequences to anneal with their respective “partners”. Thus, only 10 to 100 fold enrichment of target sequence can be obtained which has limited its use in tumor genetics.

Lisistyn et al. (14) described a technique called Representational Difference Analysis (RDA) in which 10⁵ to 10⁶ fold enrichment of target sequences can be obtained after two or three rounds of hybridization. This makes it possible to isolate minor differences such as a small deletion in complex mammalian genomes. The first step in RDA is to isolate DNA from two closely related genomes such as a DNA from normal and tumor cells from the same patient. Normal DNA (referred to as “tester”) and the tumor DNA (referred to as “driver”) are digested separately with a restriction enzyme such as Bgl II. This creates an assortment of different sized fragments. Adaptors with cohesive Bgl II ends are then ligated to the ends of the Bgl II digested fragments. These adaptors called “R” adaptors are comprised of 24mer and 12mer oligonucleotides annealed together creating a Bgl II cohesive end. After the ligation, the ends of the fragments are filled in with Taq polymerase, and then undergo 20 cycles of PCR using the 24mer oligonucleotide as the PCR primer. This 20 cycle PCR product is called the amplicon. Because PCR preferentially amplifies smaller fragments, only fragments of one kilobase or less will be amplified. Consequently, the complexity of the genome is reduced and only a “representation” of the genome remains, specifically—Bgl II fragments smaller than 1 kb.

For both the tester and driver amplicons, each are separately digested with Bgl II to remove the R-adaptors. For the tester amplicon alone, a new set of adaptors, the J-adaptors are ligated to the Bgl II cohesive ends of the tester amplicon. They are then annealed with the driver amplicon (lacking adaptors) in a 1:100 ratio. Three different populations of annealed duplex DNA form; 1) driver:driver 2) driver:tester 3) tester:tester. The ends of the annealed DNA are then filled in and PCR is performed for 10 cycles using the J-24mer as the PCR primer. In this PCR step only the tester:tester DNA duplex will be amplified exponentially. The driver:driver duplex lacks the ligated ends and will not be amplified, and the driver:tester duplex will only undergo linear amplification since only one end of the duplex contains the ligated 24mer. The PCR products then undergo digestion with mung bean nuclease to remove any single stranded DNA, and then an additional 20-25 cycles of PCR are performed. This subtraction/hybridization amplification is repeated two more times to further eliminate amplified non-specific background products. In the second and third round the process of “kinetic enrichment”, i.e. increase in the concentration of target sequences relative to non-specific background sequences that have been amplified, further enhances the enrichment of target sequences. As the relative concentration of the target sequences increases, the kinetics of annealing at the hybridization step also increases. Consequently, enrichment in the second and third round have two components-subtraction, and increased kinetic annealing rates. RDA enriches for target sequences an estimated 10⁶ fold (18).

Tumor Specimens for RDA

The selection of appropriate starting material is believed to be an important step in RDA. Extremely pure DNA samples from normal and cancer cell are needed since contaminating normal cells within a tumor may become significant when tumor DNA is in excess compared with tester DNA. Using DNA from tumor and normal tissue from the same patient, we believe, is essential to reduce polymorphic differences. If sufficient amount of genomic DNA is available, Southern bolt analysis is performed to distinguish homozygous deletions from RFLP with LOH. Thus, human tumor cells in culture are excellent specimens for RDA (19). Once a tumor suppressor gene is identified, these cells are used to carry out functional studies. Unfortunately, for most cancer cell lines, normal cells from the same patient may not be available. In such instances, freshly isolated tumor samples is an excellent source for both tester and driver DNA because resections include normal tissue surrounding the tumor. However, the tumor bed usually contains normal stroma with endothelial and inflammatory cells. Transplanting these tumors in mice i.e. heterotransplants or xenografts, for >2 passages provides a relatively pure population of cancer cells and circumvents this problem. Mouse stromal DNA contamination of the driver is not a concern in RDA. Alternatively, laser capture micro-dissection can be used to isolate a pure population of cells directly from tumor (22). The use of OTC for freezing specimens allows efficient PCR amplification after micro-dissection, which has been reported to be a problem in paraffin embedded specimens. However, the downstream analysis of subtracted clones (e.g. Southern blots) may become difficult because of a limited amount of DNA and the absence of appropriate cell lines for functional studies.

Mouse models of human cancer are a powerful tool for RDA analysis, and have not been explored to date. A number of knock out models mimic human tumors and genetic lesions such as chromosomal aberrations and LOH have been reported during tumor progression (24, 25, 26). Tumor cells can be cultured easily and we use tail DNA from the same mouse as tester. Of great importance is the absence of polymorphisms within inbred mouse strains. We believe that this feature dramatically enhances the ability to detect pathological deletions in cancer.

B. Results

RDA Analysis

Salient features in RDA are the preparation of the amplicon DNAs and hybridization of normal amplicon with tumor amplicon in a molar ratio of 1:100 followed by PCR amplification of re-annealed normal DNA sequences. Amplicons were prepared with genomic DNA from both cells in culture and xenogrrafts as described by Lisitsyn and Wigler, 1995 (19). Samples obtained from laser capture micro-dissection were prepared as described by Michiels et. al., 1998 (23).

The general scheme for evaluating the PCR fragments obtained from the 3rd round of RDA is as follows. Third round RDA products are cloned and inserts are used to probe a Southern blot containing both tester and driver amplicon DNA. Clones that hybridize to both normal and tumor amplicons represent unsubtracted background. This is variable depending on individual subtractions and background clones can be as high as 50-75% of all third round PCR products. True subtracted products i.e., a clone that hybridizes selectively to the normal DNA amplicon, can be a result of either a homozygous deletion (a complete loss from tumor genome) or more frequently a result of LOH i.e., presence of a polymorphic Bgl II restriction site in one allele combined with allelic loss (LOH) in the tumor of the Bgl II site present in the second allele. These LOH products are distinguished from homozygous deletions on Southern blots using genomic DNA. Alternatively, the subtracted clone is sequenced and used to design PCR primers allowing amplification of genomic DNA from tumor cells in LOH cases but not in cases with homozygous deletions. This method is rapid, reliable and requires only a minute amount of genomic DNA. Therefore, it is suitable for laser capture micro-dissected specimens. RDA was successfully performed on several human tumors samples derived from cells in culture, tumor xenografts grown in nude mice and laser capture micro-dissected specimens. Wilms tumor, ovarian carcinoma and renal cell carcinoma results are summarized in Table 1.

TABLE 1
Summary of RDA results on human tumors

| | | | NUMBER OF |
|---|---|---|---|
| TUMOR | | NUMBER OF | HOMOZYGOUS |
| SPECIMEN | | LOH | DELETED |
| TYPE | SAMPLE | PRODUCTS | PRODUCTS |
| Cultured cell lines | Renal Cell | 5 | 12 |
| | Carcinoma | | |
| | Wilms 96W | 5 | 1 |
| Tumor xenografts | Wilms #8 | 6 | 0 |
| | Wilms #14 | 8 | 0 |
| | Wilms #11 | 1 | 0 |
| | Wilms #15 | 2 | 0 |
| Laser Capture | Ovarian #1 | 8 | 1 |
| Micro-Dissection | Ovarian #2 | 2 | 0 |
| | Ovarian #3 | 0 | 0 |
| | Ovarian #4 | 5 | Not determined |
| | Wilms #7107 | 1 | 0 |

Subtracted products validated by amplicon blots were seen in 10 of 11 tumor samples (ovarian carcinoma # 3 had none) implying that RDA worked in nearly all cases studied. The number of probes tested for subtraction from each tumor sample ranged from 4 to 20. PCR products representing homozygous deletions were fewer than the LOH products except in the case of renal cell carcinoma cell line. LOH products were not analyzed further.

Evaluation of Homozygous Deletions

Clones representing homozygous deletions were likely to contain a tumor suppressor gene. The chromosomal location of these clones were mapped by using one of the following: genomic database search, gene specific PCR on a radiation hybrid panel or on mouse/human somatic cell hybrids and FISH. Of the twelve probes representing homozygous deletions in the Renal Cell carcinoma cell line, five mapped to Xq22-q26. This was a very large deletion (>10 Mb) and was not further pursued given the large number of genes within this locus. The remaining 7 probes all mapped to the locus at chromosome 9p that contain the p16^{ink4A} tumor suppressor gene, known to be frequently deleted in human cancers. Although not novel, this result confirmed our ability to identify a homozygous deletion in a tumor suppressor gene locus.

PCR product representing a homozygous deletion in Wilms tumor cell line 96W mapped to 6q21 by FISH. Further characterization of the deletion revealed that it represents a common polymorphism in the population (allele frequency: 0.41, homozygous deletion frequency: 0.17). Similarly, PCR products representing homozygous deletion in laser capture micro-dissected ovarian carcinoma sample #1 was analyzed. It mapped on chromosome 17q22, and a small, <2 Kb, homozygous deletion was identified. Remarkably, this region also proved to represent polymorphic deletion in the human population (allele frequency: 0.17, homozygous deletion frequency: 0.03.

As evident by these results, even if performed on optimal human specimens, frequent polymorphic deletions were present in the human population. Unlike restriction enzyme polymorphisms, these are not readily excluded by RDA results, and require extensive analysis before they can be discarded (21). To circumvent the complications associated with human polymorphisms, we elected to work with the mouse cancer models using syngeneic strains. Our focus on mouse tumor suppressor loci allowed us to overcome the above described complications associated with human polymorphisms.

RDA in Mouse Tumor Models

RDA on an inbred system, such as mouse tumor cell lines, may decrease the probability of isolating polymorphic deletions. Mouse tumors accumulate sequential genetic lesions as seen in humans: mutations and allelic loss affecting both p53 and RB have been reported as have homozygous deletions of p16 (25, 26, 27). Loss of p53 in the mouse knockout model reportedly causes genetic instability leading to a variety of tumor types (28). This represents an optimal starting point in the selection of mouse tumor samples for genome wide analysis to isolate novel genes implicated in cancer progression.

We previously have shown that mice lacking both p53 and NF2 develop large tumors, primarily osteosarcomas, fibrosarcomas and hepatocellular carcinomas (29). NF2 and p53 are present on the same chromosome in the mouse, and NF2/p53 double heterozygotes survive for only five months before developing multiple tumors with LOH spanning both NF2 and p53 loci (29). Tumors in mice usually do not show aggressive behavior or metastasize, whereas tumors driven by loss of both p53 and NF2 are highly invasive and metastasize (29). We have cultured cells isolated from primary and metastatic tumors and have used those cells in our experiments.

As a first mouse experiment, DNA was isolated from two cancer cell lines (osteosarcoma 3442 and fibrosarcoma 3085, both are derived from p53/NF2 heterozygous mice) and tail DNA from SV129. A total of 18 subtracted clones were obtained from these cell lines after two rounds of RDA. All 18 clones were tested by PCR for homozygous deletions. All 18 clones represented restriction fragment length polymorphisms. This result shows that like human tumors there indeed is LOH in mouse tumors. Polymorphisms arise in what is believed to be an inbred population like laboratory mice after several generations.

To minimize population effects, tail DNA from the same mouse from which the tumor cell line was derived was used in RDA. The results from seven matched tail experiments are summarized in Table 2.

The genetic makeup of cancer cell line 3452 is as follows: the p53 and NF2 knock out alleles are present in trans on mouse chromosome 11. Products after three rounds of RDA were cloned and two of the four clones (#1 and #4) were subtracted on amplicon blots. These clones were then tested on a mouse Southern blot with normal tail DNA and DNA from four cancer cell lines digested with Bgl II. The blot was probed with both clones #1 and #4. Since genomic DNA was digested with Bgl II for both RDA and Southern blot, a band of the same size as the probe is expected and is seen in normal DNA and DNA from two cancer cell lines 3081 and 3678. No hybridization is seen with tumor DNA 3452. A similar result was seen with clone #4 confirming that both clones #1 and #4 are homozygous deletions in tumor 3452. Sequencing and blast search for these clones did not show any matches on the sequence database. Radiation hybrid mapping was performed to determine the chromosomal location of these clones. Clone 1 mapped to a marker, D4MIT300 on mouse chromosome 4 close to the interferon locus and Cdk2na locus. The Cdk2na locus encodes for the tumor suppressor gene p16 and deletions of these genes are commonly seen in several tumor types. These deletions often tend to be large and it would not be surprising that in tumor 3452 there is a large deletion, which inactivates p16 and the surrounding marker D4Mit300. To confirm this hypothesis, the presence of p16 exon 2 was tested in cancer cell line 3452. A p16 specific, 300 bp band is seen when tail DNA is used from SV 129 in PCR. Similarly, DNA from cancer cell lines 3081, 3775 and 3085 amplify a p16 specific band. When tail DNA from 3452 is used p16 amplification is seen, however, p16 is not amplified when DNA from cancer cell line 3452 is used. This suggests that there is a somatic loss of p16 in genome of tumor 3452. Since p16 is an established tumor suppressor gene, tumor cell line 3452 was not pursued any further. The results confirmed our hypothesis that mouse RDA is an optimal technique for isolating homozygous deletions leading to the identification of tumor suppressor genes.

TABLE 2
Summary of RDA results on mouse tumors with matched tails

| | | Number of | |
|---|---|---|---|
| | | deleted | |
| Tumor cell line | Sex | products | Result |
| 3452 | male | 2 | Deletion of p16 |
| 3081 | female | 4 | Deletion of a novel gene |
| 3442 | female | 0 | No deletions |
| 3085 | female | 0 | No deletions |
| 3872 | male | 6 | Y chromosome loss |
| 3775 | female | 4 | Not determined |
| 3678 | female | 0 | No deletions |

Deletion in Osteosarcoma Cell Line 3081

The next question to answer was that can novel tumor suppressor genes be identified using mouse RDA approach? The results from previous experiments show that not all NF2/p53 derived cancer cell lines lose p16 and therefore could be suitable for RDA to identify novel genes. DNA from an osteosarcoma cell line 3081. No hybridization of the probe is seen with Bgl II digested DNA from tumor 3081. Radiation hybrid mapping shows that Clone 11 is located close to a marker D12Mit148 on mouse chromosome 12. No known tumor suppressor genes are located on mouse chromosome 12. The deleted clones were sequenced and used for blast homology search of the high throughput genome sequence database where work in progress sequences from mouse and human genome are posted. Clone 11 mapped to mouse BAC clone AC 079370 and clone 13 mapped to a mouse BAC AC 079369. Both BACs mapped to mouse chromosome 12 and covered approximately 500,000 base pairs. The exact distance between the clones 11 and 12 was not determined since the BAC sequences are currently unordered.

To approximate the extent of the deletion in tumor cell line 3081, the markers surrounding D12Mit148 on chromosome 12 were examined. On the 5′ end of BAC AC 069370, a Zinc finger gene ZNF277 is present. A probe specific for this gene was used in a Southern Blot on Bgl II digested cancer cell line DNA. The results confirm that the ZNF277 gene is present in cancer cell line 3081. Similarly, the DLD (dihydrolipomate dehydrogenase, an E3 enzyme involved in the Kreb's cycle) gene is present towards the 3′ end of the deletion (3′ to BAC AC 069369). Its presence was ascertained by PCR. A 500 bp band specific to DLD gene is amplified from all three tumors confirming the presence of DLD in tumor 3081. The DLD gene is several hundred thousand base pairs from BAC 069369 and the sequence data from this region is currently unavailable on public databases. We believe that there are possibly 5-6 BACs between the two regions. Further blast searches revealed that BAC 084316 is just 3′ to BAC 069369 and a probe specific to it was used for Southern blot analysis. There is no hybridization of the BAC 084316 specific probe to tumor DNA 3081 suggesting that it is included in the genomic deletion on chromosome 12. We have not been able to further refine the ends of the deletion but it is clear from our data that the deletion on chromosome 12 in osteosarcoma cell line 3081 involves at least three BACs spanning a region of >500,000 bp.

From Deletion to a Novel Candidate Tumor Suppressor Gene

Using blast search, we identified a DNA contig on human chromosome 7 which is homologous to the deletion on mouse chromosome 12. Human 7q31 is extensively involved in LOH in several tumor types. We embarked on the positional cloning of the gene on human 7q31, focusing on the region, which is, homologous to the deletion in mouse osteosarcoma cell line 3081.

The human 7q31 contig is sequenced and therefore can be used as input data in gene prediction programs. Using such programs, we were able to predict a transcript made by fusion of several exons spanning about 500,000 bp genomic DNA. The start of transcription was predicted at BAC AC 003077 and terminates at BAC AC 005047. The 3′ end was confirmed by searching the EST database, which revealed several matches from multiple tissue types. These ESTs are 3′ end sequence reads from libraries that were made by oligo-dT priming method, thus they represent 3′ ends of transcripts. Using RT-PCR with primers from predicted exons we were able to amplify the predicted cDNA. Since, gene prediction programs are not able to predict the 5′ ends of transcripts, we performed RACE using a human placenta RACE library and reverse primers from the predicted 5′ end. The RACE results show that the transcription of this gene starts at BAC AC004111.1. This approach has enabled us to identify a novel gene, which is referred to as DOS for Deleted in Osteosarcoma. DOS is located on chromosome 7q31, with ZNF277 being telomeric while the DLD gene centromeric to it. The BAC AC 004001.1 is entirely an intron, and contains the LOH marker D7S523. A tissue Northern blot with a DOS probe shows that a transcript of 8.5 Kb is multiple tissue types. The level of expression is low and can be detected by RT-PCR from several different tissue types. The DOS mRNA encodes for a large protein of 1966 amino acids with predicted size of 225 KD. DOS has sequence homology to three proteins in the database, namely human DOCK180, Drosophila myoblast city and C. elegans Ced-5. These proteins are conserved in sequence and in function. They reportedly are involved in the initiation of cell migration by triggering actin polymerization at the cell edge via small GTPase Rac1. Therefore, these proteins have been reported to have a role in several fundamental biological processes such as morphogenesis, cell migration during development and engulfment of apoptotic cells. DOS, like DOCK180, has an N terminal SH3 domain and two conserved PFAM B (Sanger Centre, Cambridge, UK) domains namely, 9939 and 2818, suggesting extensive sequence identity. Additionally, there are regions in DOS where sequences are not homologous to DOCK180 such as the C terminal 300 amino acids. Both proteins have proline rich regions but these regions do not align with each other. We believe that DOS is involved in the regulation of actin polymerization, but is distinct in function from DOCK 180, and exhibits other functional activities that are related to its roles as a tumor suppressor gene and/or regulator of cytoskeletal organization, cell growth, cell proliferation, cell migration, and/or cell-cell interactions.

Osteosarcoma cell line 3081 was derived from a primary tumor in an NF2/p53 heterozygous mouse. Our data show that DOS is deleted in this particular cell line. It is likely that DOS will be targeted by mutations in other tumor isolates arising in the same genetic background since they have similar temporal relationship to tumor progression. If DOS is involved in cancer progression in general, it will be targeted by tumors of different genetic backgrounds.

Screening for DOS Mutations in Mouse NF2/p53 Cancer Cell Lines

About 50 cell lines derived from tumors developed in mice heterozygous for tumor suppressor genes p53 and NF2 and both NF2 and p53 have been isolated. Sequence analysis of DOS in these three different backgrounds is performed to determine the relative contributions of p53 and NF2 loss. Mice that are heterozygous for NF2 alone form tumors at about 9 months age. Mice heterozygous for both NF2 and p53 develop aggressive tumors and die by three months age. This is presumed to be due to the “genomic instability” acquired after p53 loss. Therefore, we believe there are more mutations in DOS when NF2 loss is coupled with p53 loss.

Screening for DOS Mutations Metastatic Verses Primary Cell Lines

We selected NF2/p53 tumor cell lines that have been derived from primary tumors and an equal number of cell lines derived from metastatic tumors to confirm that DOS is involved in tumor progression. Assuming that DOS functions as a tumor progression gene, we anticipate observing a higher frequency of mutations in DOS in metastatic cell lines.

Identification of the Boundaries of the Deletion in Tumor Cell Line 3081

Our results show that the 5′ end of the deletion is near the start of the DOS gene. Monthly homology searches are performed to find DNA segments 3′ to the DOS gene and 5′ to the DLD gene and, thereby, further define the boundaries of this deletion. These segments are tested on mouse Southern blots. This assists in cloning the breakpoint and determines the contribution of such a genetic loss in tumor biology.

D. Methods to Screen for Mutations in DOS in Human Cancer

Introduction and Hypothesis

Allelic losses in tumors are typically detected as “loss of heterozygosity” or “LOH”. While LOH is a common event in cancer, it only allows rough mapping of tumor suppressor loci (9). However, these studies do suggest that a tumor suppressor gene is nearby the LOH marker. Human DOS is located on 7q31, a region observed to have LOH in multiple tumor types including ovarian, prostate and breast cancer. A study on 22 primary ovarian cancers with 16 microsatellite markers showed that the minimal region of LOH was at 7q31.1 involving marker D7S523. Based on mouse deletion of DOS and human LOH studies, we believe that DOS is a tumor suppressor gene and is targeted by mutations in several tumor types.

Sequencing of DOS cDNA in Human Cancer Cell Lines

Available to us are a panel of human cancer cell lines obtained from either ATCC or NCI. Multiple samples are run in a very short period of time on Applied BioSystems 3100 Genetic analyzer (Applied Biosystems, Foster City, Calif.). Direct sequencing of RT-PCR fragments is the method of choice as DOS is a large gene with several small exons (average size 100 bp), therefore genomic DNA sequencing is not feasible. We perform primary RT-PCR using primers spanning the entire open reading frame of DOS with cDNA from various cancer cell lines. These primary PCR reactions are diluted, then used in secondary PCR reactions. A total of 13 secondary PCR reactions with an average size of 600 bp are required to accurately analyze every base pair of the open reading frame. Sequencing is performed on both strands using Dye-Terminator (Applied BioSystems, Foster City, Calif.) since we are primarily interested in homozygous changes. We anticipate two classes of mutations, one leading to premature chain termination and the other causing missense mutations. Premature termination almost always undoubtedly causes loss of function in gene expression and such results further validate DOS as a tumor suppressor gene. Missense mutations are more difficult to interpret since they represent single nucleotide polymorphism. The region of missense mutation in DOS from normal controls is sequenced eliminate this possibility. DNA and RNA from EBV immortalized normal peripheral leukocytes also are used in such studies. Missense mutations are likely to target conserved amino acid residues in mouse and human DOS. Search for mutations in cancer cell lines have several advantages in that cells derived from multiple tumor types can be screened rapidly. RNA from cell lines is relatively pure, free from contaminating normal tissue.

Screening for Mutations in DOS in Primary Human Tumors

A large tumor bank with most tumor types is available for our genetic studies. For most tumors, matched normal samples are available. Since DOS is a large gene, RNA isolated from laser capture micro dissected material is not particularly useful for RT-PCR. Surgical resections is the method of choice. Tumors are dissected from surrounding normal tissues and RNA is isolated using STAT-60. Primary and secondary RT-PCR reactions for sequencing are performed. If a mutation is seen in a particular sample, the matched normal is also sequenced in the same region. Sequencing normal genetic material from the same patient shows that the mutation is an acquired sporadic event.

Screening for Mutations in DOS in Human Primary Verses Metastatic Tumors

Whether these mutations are acquired early or late in carcinogenesis is next determined. For example, following identification of mutations in DOS in prostate cancer, the mutations are correlated with grade, stage and metastatic disease. Such results are consistent with a frequency of LOH at 7q31 in prostate cancer is higher in late stage tumors and correlates with aggressive behavior and metastasis.

E. Method for Biochemical and Functional Characterization of DOS

Introduction and Hypothesis

Initiation of cell migration is characterized by rapid actin polymerization to the migrating cell's leading edge. The result is a protrusion of a lamellipodium with membrane ruffles observed at the cell surface. These ruffles serve as sites for actin polymerization, endocytosis, protease activation and receptor tyrosine kinase signaling. Genetic and biochemical studies have shown that membrane ruffling requires at least three proteins, namely, Rac1, DOCK180 and CrkII. The protein DOCK180 was originally identified by its ability to interact with adaptor protein CrkII. It is now believed to signal downstream the integrin receptor αᵥβ₅ and initiate cell migration. In C elegans, there are at least six genes identified in engulfment of apoptotic cells; three of these genes, Ced-2, Ced-5 and Ced-10 are a part of one complementation group. Ced-5 codes for the ortholog of DOCK180, Ced-2 codes for ortholog of CrkII and Ced-10 encodes for Rac1. Myoblast city, a homologue of DOCK180 in Drosophila has been shown to be a mediator of Rac1 activity in several morphogenetic activitites during drosophila embryogenesis, including myogenesis, neural development and dorsal closure. Thus, these proteins are a part of complex biochemical signaling events leading to cell migration and loss of adhesion. We believe these proteins are involved in the process and several components in this pathway are yet to identified. We have a found a gene which has significant sequence identity to DOCK180. Accordingly, we believe that DOS is a component that, directly or indirectly, regulates small GTPases and actin polymerization. Since DOS genetically exhibits a loss of function in a mouse tumor, we believe that one of the DOS functional activities is as a negative regulator of actin polymerization and cell migration. Additional DOS functional activities are directed to tumor invasion and metastasis.

Tissue Expression of DOS by Northern Blots and in situ Hybridization

DOS tissue expression at the RNA level using Northern blots (Clontech, Palo Alto, Calif.) is assessed. These blots are probed with cDNA fragments from the 3′ and 5′ end to identify alternate splicing. In the mouse, developmental stages are assessed by Northern analysis. We have shown by RT-PCR that DOS is ubiquitously expressed. It is not surprising that DOS is present in every tissue because it is likely to be involved in fundamental processes such as, but not limited to, actin polymerization. DOS-specific, small RNA probes for in situ hybridization also are prepared and used to assess mouse developmental stages.

Generating Epitope Tagged Expression Vectors for DOS and DOS Mutants

Epitope tagged, full length construct for eukaryotic expression is preferably made in pCDNA3.1/V5-His TA TOPO vector (Invitrogen, Carlsbad, Calif.). This vector offers several advantages including versatile expression driven by CMV promoter and detection of the fusion protein with V5 antibody or His antibody. DOS cDNA is amplified in 2 Kb fragments by RT-PCR using PwoI DNA polymerase for low error rates. N-terminal SH3 domain and C-terminal proline rich domain deletion constructs are made. Detection of fusion proteins on Western blots is optimized.

Preparation of Antibodies to DOS

Polyclonal antibodies to the DOS protein are made for immunoprecipitation and immunofluorescence studies by cloning the C terminus into vector pGEX2T. The C-terminus has no homology to DOCK180 and is virtually identical in human and mouse DOS which makes it a good choice for antibody production. Antibodies are affinity purified and tested for detection of native DOS in both mouse and human cells.

Expression and Sub-Cellular Localization of DOS Protein in Osteosarcoma Cell Line 3081

Studies on DOS protein expression are performed after transfection of epitope tagged constructs in osteosarcoma cell line 3081. This mouse tumor cell line has no endogenous expression of DOS because of DOS gene deletion. After transient transfection of the full length and mutant constructs in 3081 cell line, its effects on cell growth, proliferation and cell shape are determined using conventional assays. Cells are stimulated by growth factors such as EGF, PDGF, FGF and insulin. Immunofluorescence studies with anti-epitope antibodies are used to determine DOS sub-cellular localization.

A number of studies have reported that activation of Rho GTPases requires membrane translocation. Transfection of wild type DOCK180 in NIH 3T3 cells show its accumulation in the cytoplasm. When DOCK 180 was fused at the C-terminus with farnesylation signal of ras, DOCK180 localized mostly to the membrane. There was a change in shape of the fibroblasts to a pancake morphology which is suggestive of activation of Rac1. In the case of transient transfection studies with DOS, it is conceivable that there is cytoplasmic staining in quiescent cells. The effect of growth factor receptors such as EGF, FGF, PDGF and insulin stimulation on cell shape and/or DOS localization is determined. Studies on cell shape and/or DOS localization are done after activation of integrin mediated signaling by plating cells on vitonectin or fibronecton and activation of non-integrin mediated cell adhesion by plating cells on polylysine. Farnesylation signal also is fused at the C terminus of DOS to mimic DOS activation and the effect on cell shape and/or DOS localization/activation is determined.

Effect(s) of Rho, Rac and CDC42 on DOS

Following the selection of a particular DOS expression phenotype, dominant negative constructs for Rho, Rac and Cdc42 are used in cotransfection studies to determine whether specific inhibitors of small GTPases affect the selected DOS function. Antibodies to DOS are used in immunofluorescence studies with confocal microscopy to assess DOS association with focal adhesion complexes, actin-myosin stress fibers, membrane ruffles, lamellipodia and/or filopodia. These structures are specific to activation of small GTPases.

Regulation of small GTPases occurs at the level of nucletotide exchange and is mediated by three distinct proteins families: 1) GEFs or guanine exchange factors which activate GTPases in response to extracellular signals, 2) GAPs which inhibit GTPases by maintaining it in predominantly GDP bound form, and 3) GDIs which bind to the GDP bound form and prevents both spontaneous and GEF mediated release of GDP. Thus, there seems to be an exquisite control for the activation of small GTPases. Co-transfecting DOS with either GEFs or GAPs is performed to assess the role of DOS in GTPase activation.

Protein Association Studies with DOS

Protein association in signal transduction often involves organization of multi protein complexes via modular components such as SH2, SH3 and PTB domains. SH3 domains bind proline rich peptides and several SH3 domains and their cognate proline rich sequences have been established. These interactions are involved not only in protein association but often target proteins to different sub-cellular destinations. DOCK180 has two consensus Crk SH3 binding sites in the C-terminus that are critical for the formation of the integrin mediated CrkII-DOCK180-Rac1 complex. DOCK180 and DOS have no significant sequence homology at the C-terminus. DOS shows sequence homology to 3BP1, a mouse protein that associates with Src, Abl and Grb2 via SH3 domains. Therefore, the leading candidates for DOS C-terminal binding are Src, Abl and Grb2. Although not wishing to be bound to any particular theory or mechanism, we believe that DOS has at least 3 functional domains which mediate its biological function:
C-terminus (amino acids 1701 to 1966) putative SH3 binding sites.
N-terminus (amino acids 10 to 100) an SH3 domain.
Region of homology to DOCK 180 (amino acids 110 to 1100) putative small GTPase binding site.

Protein binding studies require cloning the individual domains in GST fusion vectors. GST pull down assays have been used successfully to study the interaction of SH3 domains with proline rich residues. GST fusion proteins are incubated with cell lysates and associated proteins resolved by poly acrylamide gel electrophoresis and transferred to nitrocellulose membranes. Bound proteins are identified by Western blotting with specific antibodies to signaling proteins. Alternately, GST-fusion proteins from Crk, Src, Abl, Nck and Grb2 are incubated with cell lysates expressing epitope tagged full length and C-terminus deletion DOS constructs. Antibody to the epitope is used to detect binding of DOS to its cognitive SH3 domain. Also, DOS SH3 domain is used in such experiments. DOS N terminal SH3 domain may interact with DOS C-terminus leading to both intra and intermolecular interactions. Alternately, the SH3 domain binds to another molecule, thereby, affecting the sub cellular localization of DOS.

DOCK180, when cotransfected with the Rho specific GEF Vav, seems to increase Rac activity. This is believed to be due to the ability of DOCK180 to bind to nucleotide free Rac, an intermediate in guanine exchange reaction catalyzed by Vav. Using GST fusion proteins for Rho, Rac and CDC42, the binding for DOS is assessed. Binding of DOS to small GTPase may require addition of nucleotides GDP or GTP-γ-S. Since, there are differences in amino acid sequence, it is possible that DOS may interact with Rho or CDC42 but not Rac which has shown to be able to bind DOCK180.

The invention is not limited by the proposed mechanisms and/or theories disclosed herein. The above described experiments are based, in part, on the functional domains present in DOS that are disclosed herein; however, predicted interactions may not be observed experimentally for various reasons. Accordingly, additional experiments such as a more conventional yeast two hybrid approach is used to confirm one or more of the DOS functional activities disclosed herein. In this approach, individual domains of DOS are used as baits to clone interacting proteins. The binding of these proteins is further characterized in vivo in mammalian cells.

Physiological Studies on DOS

Experiments designed to address the physiological role of DOS expression in cells are determined by results from biochemical analysis and identification of the major signaling pathway. Since DOS was cloned as a deletion in tumor, we believe that loss of function of DOS offers survival advantage to tumors. The following experiments directed to tumor invasion and angiogenesis assays are performed to assess this DOS functional activity. Briefly, osteosarcoma cell line 3081, which has a deletion in DOS is transfected with full length and C terminal mutant DOS. Stable cell lines are generated and Western blots are done to assess the expression of DOS proteins. These cells are injected subcutaneously and mice are observed daily and sacrificed one week after injection. Tumors are fixed and sectioned and studied for their invasiveness. Similarly, angiogenesis is studied by sacrificing mice six weeks after injection. To study invasion, a G8 myoblast layer is fixed in six well plates. These layers are seeded with stable transfected 3081 cells. After one week, cells are observed for invasion by microscopy. Such experiments require optimal controls and are repeated with different isolates of stable transfectants. Following observation of a phenotype in these assays, different molecular components of DOS are further tested. In addition, other cell migration experiments are considered such as the Transwell migration assay with transfected COS-7 cells. This assay has been particularly useful in the identification of the Crk/Cas complex as a key switch in the initiation of cell migration and actin cytoskeleton reorganization. Transwell migration assay with COS-7 cells is more amenable to experimental manipulation and, therefore is used to further characterize the molecular events underlying in DOS functional activities.

Role of DOS in Human Tumorigenesis

Human cancer cell lines were screened for DOS mutations and missense mutations in the coding sequence of DOS were found in those cell lines. Tables 3 discloses exemplary Mutant DOS molecules. Data show the nucleotide position, and the nature of the nucleotide change in the human DOS nucleic acid molecule (SEQ ID NO: 1) and the corresponding amino acid change in the human DOS protein (SEQ ID NO: 2) generated by the mutations. To illustrate, SEQ ID NO: 5 refers to a guanine to adenine substitution at position 5650 in the human DOS nucleic acid molecule (SEQ ID NO:1); SEQ ID NO: 6 refers to the corresponding valine to methionine substitution in the human DOS protein (SEQ ID NO:2).

TABLE 3
DOS mutations in human cancer

| | | | | | | | | | | |
|---|---|---|---|---|---|---|---|---|---|---|
| | | | | SEQ | | Amino | | SEQ | Effect | |
| Cell | Tissue | Nuclcotide | | ID | | Acid | | ID | on | Freq. in |
| Line | Origin | Position | Mutation | NO: | Status | Position | Mutation | NO: | Protein | normals |

Mutations

| | | | | | | | | | | |
|---|---|---|---|---|---|---|---|---|---|---|
| U251 | CNS | 5650 | G to A | 5 | heterozygous | 1884 | Val to Met | 6 | missense | 0 of 187 |
| SNB19 | CNS | 5650 | G to A | 7 | heterozygous | 1884 | Val to Met | 8 | missense | 0 of 187 |
| HCT15 | colon | 260 | C to T | 9 | heterozygous | 87 | Thr to Ile | 10 | missense | 0 of 190 |
| HCT15 | colon | 3325 | A to C | 11 | heterozygous | 1109 | Lys to Thr | 12 | missense | 0 of 187 |
| HCT15 | colon | 5263 | T to C | 13 | heterozygous | 1755 | Ser to Pro | 14 | missense | 0 of 190 |
| OV1063 | ovarian | 5153 | C to T | 15 | homozygous | 1718 | Pro to Leu | 16 | missense | 0 of 190 |
| DU145 | prostate | 5153 | C to T | 17 | homozygous | 1718 | Pro to Leu | 18 | missense | 0 of 190 |

Polymorphisms

| | | | | | | | | | | |
|---|---|---|---|---|---|---|---|---|---|---|
| HT29 | colon | 5198 | C to G | 19 | heterozygous | 1733 | Pro to Ala | 20 | missense | 2 of 134 |
| MDA MB 231 | breast | 5777 | C to T | 21 | heterozygous | 1926 | Ser to Leu | 22 | missense | 4 of 63 |
| HCT116 | colon | 5750 | C to T | 23 | heterozygous | 1917 | Pro to Leu | 24 | missense | 2 of 63 |
| common | common | 5740 | G to A | 25 | — | 1914 | Val to Ile | 26 | missense | nd |
| MDA MB 145 | breast | 1816 | G to C | 27 | heterozygous | 606 | Glu to Gln | 28 | missense | 7 of 38 |

### Example 2

Introduction

We have found missense mutations in established human tumor cell lines derived from breast, prostate, ovarian and colon cancers. Like other CDM family members, DOS binds to the tyrosine phosphorylated adaptor protein, Crk. DOS harboring a proline mutation identified in human cancer does not bind to the adaptor protein Crk. DOS is unique since it is the only CDM family member that binds to c-Src and rescues the phagocytosis defect seen in ced-5 mutants C elegans. In addition, reconstitution of expression of DOS in the DOS null osteosarcoma cell line, altered cell shape and cells were contact inhibited. Therefore, our data support the hypothesis that DOS is a candidate tumor suppressor gene that appears to function as a bottleneck in cellular pathways responsible for modulation of actin cytoskeleton during morphogenesis, phagocytosis and cancer.

In order to understand how DOS functions in such fundamental cellular pathways we took the opportunity to compare the biological properties of DOS null osteosarcoma cells with cells reconstituted with either wild type or mutant DOS identified from human tumor cell lines. Understanding the protein network centered on DOS will help elucidate its function in cell biology, cancer and potentially identify novel therapeutic targets in oncology.

Background

The study of cancer cells in culture have led to the description of the “transformed phenotype” which alludes, in part, to the alterations in serum and adhesion dependant growth properties of cells. Furthermore, in transformation there were changes in cell morphology and these transformed cells exhibited loss of contact inhibition with uncontrolled cell migration. A vast majority of these changes have been attributed to alterations in the actin cytoskeleton. The small GTPases, Rho, Rac and CDC42, are believed to be responsible for the formation of actin mediated assemblies such as stress fibers, focal adhesions, lamellipodia and filipodia, in both normal and transformed cells. RDA on optimal human cancer specimens in our laboratory showed frequent polymorphic deletions in the human population. Since inbred mouse models of human cancers lack genetic polymorphism, we hypothesized that RDA on mouse tumor samples should dramatically enhance the ability to detect pathological deletions in cancer.

Using mouse RDA, a homozygous deletion was identified on mouse chromosome 12 in osteosarcoma cell line, 3081. This region is syntenic to a sequenced contig on human chromosome 7q31 and therefore could be used as input data for gene prediction programs. This approach has enabled us to identify a novel gene, which is referred to as DOS for Deleted in Osteosarcoma. DOS is located on chromosome 7q31near the LOH marker D7S523 which is frequently lost in advanced ovarian cancer. The deletion on mouse chromosome 12 was large but presumably only inactivates DOS because the predicted 5′ and 3′ genes, ZNF277 and DLD respectively, were intact based on Southern blot analysis.

DOS transcript is of 8.5 Kb in size and was universally expressed at low levels based on Clontech multiple tissue Northern blot. The DOS mRNA encoded for a large protein of 1966 amino acids (SEQ ID NO: 2)with predicted size of 225 KD. We have also identified an alternatively spliced form of DOS, which lacked an exon encoding 38 amino acids (SEQ ID NO: 32) very close to the C-terminus of the protein. This spliced out region contained the tyrosine kinase, c-Src SH3 domain binding consensus amino acid sequence PPVPPR. We, refer to the larger protein of 1966 amino acids as DOS+SSB (SEQ ID NO: 2) (with Src SH3 domain binding site) and to the smaller 1928 amino acid protein as DOS−SSB (SEQ ID NO: 30) (without Src SH3 domian binding). Using RT-PCR we observed that cells growing in culture express DOS+SSB only (>50 cell types tested). Expression of DOS−SSB can be observed when RT-PCR is performed on RNA derived from whole organs. It is likely that cells express only one isoform of DOS and the study of differential expression of the two isoforms in an organ by in situ hybridization helps understanding the function of DOS.

DOS is Mutated in Human Cancer

The human DOS gene is located on the locus 7q31. This locus is frequently involved in LOH in solid tumors of various histologies including breast, prostate renal and colon. As a preliminary screen to search for mutations in human cancer, thirty five established cell lines from breast, colon, ovarian and renal cancer available through either ATCC or NCI, were sequenced. A total of five cell lines derived from ovarian, prostate, brain and colon cancer were found to have missense mutations in DOS. The ovarian (OV1063) and prostate (DU145) cancer cell lines carry the same homozygous, missense mutation, changing the proline residue at amino acid 1718 to leucine (SEQ ID NO: 16 and SEQ ID NO: 18 respectively). The colon cancer cell line has a total of three missense mutations (SEQ ID NOs: 9, 11, and 13) in the DOS coding sequence. In this limited screen we have not found inactivating mutations in DOS. The missense mutations observed are unlikely to be polymorphisms since these changes were not found in 190 normal individuals. Also, the proline to leucine mutation showed a loss of binding to adaptor protein, Crk. These results support the hypothesis that DOS is a novel tumor suppressor gene.

Cloning of DOS

Using conventional positional cloning techniques, we were able to clone both, DOS+SSB and DOS−SSB in pcDNA3.1 under the CMV promoter. Also available, is the DOS+SSB with the proline to leucine mutation referred to as DOS+SSB P>L found in ovarian cancer cell line (SEQ ID NO: 16) and prostate cancer cell line (SEQ ID NO: 18).

DOS is a Novel Member of CDM Family of Proteins

DOS has sequence homology to four proteins in the database, namely human DOCK180, DOCK2, Drosophila myoblast city and C. elegans Ced-5. These proteins are conserved in sequence and in function. They are believed to be involved in the initiation of cell migration by triggering actin polymerization at the cell edge via small GTPase Rac1. Therefore, these proteins have been shown to have a role in several fundamental biological processes such as morphogenesis, cell migration during development and engulfment of apoptotic cells.

Based on sequence homology and functional studies, CDM family members seem to have evolved from the C elegans gene, ced-5. This gene along with ced-2 (CrkII) and ced-10 (Rac) were initially identified to play a role in phagocytosis of dying cells during programmed cell death. A second defect in pathfinding during the third phase of distal tip cell migration was later also observed in these ced mutants. Along with DOS there are two other human genes in the database that are CDM family members, and have also evolved from ced-5, namely, DOCK180 and DOCK2. The protein DOCK180 is universally expressed in all cell types except for lymphocytes, while DOCK2 is only expressed in lymphocyte. DOCK180 activates Rac leading to membrane ruffles and affects cell morphology and cell migration. It also rescues the ced-5 distal tip cell migration defect without affecting phagocytosis of dead cells. DOCK2 also activates Rac and plays a critical role in T and B cell migration. In contrast, our data showed that the rescue of ced-5 with DOS+SSB reversed the defect in phagocytosis of dead cells without affecting distal tip cell migration. DOS+SSB with proline to leucine mutation did not rescue either phagocytosis or distal tip cell migration suggesting that in this assay it is a loss of function allele. DOS−SSB, a splice variant also did not rescue the Ced 5 phenotype. With these results in mind, one can envision the possibility that during evolution, Ced-5 has diversified into at least three mammalian genes; DOCK180 and DOCK2, retain the regulation of cell migration function of Ced-5, while DOS retains the regulation of phagocytosis function of Ced-5.

SH3 Domains of Src and Crk Interact with DOS

In transient expression studies, with epitope tagged DOS constructs in both 293T cell and COS cells, a protein of the expected size is readily detected after cell lysis with buffers containing detergents, such as RIPA.

After incubating epitope tagged DOS lysates in SDS containing RIPA buffer with beads carrying GST-Crk-N terminal SH3 domain, DOS+SSB and DOS−SSB was “pulled down”. However, DOS+SSB with proline mutation was not “pulled down” suggesting a decreased binding to Crk. Similarly, GST-Src SH3 domain showed efficient binding to DOS+SSB. No binding to DOS−SSB was observed. DOS−SSB does not contain the sequence PPVPPR, the putative Src SH3 domain binding sequence, hence, does not bind to Src. Notably, there was decreased binding of DOS with the proline mutation. The proline rich, C terminus of DOS engages with Src and Crk and the loss of Crk binding is seen in human cancer. Furthermore, no binding to SH3 domain of Abl and Crk-C terminus was observed.

Cell Growth, Morphology and Actin Cytoskeleton Studies

After transient transfection of several established cell lines including fibroblast cell lines such as NIH3T3 or in 3081, DOS null, osteosarcoma cells, we were unable to detect DOS protein expression Therefore, with the aim to pursue functional studies, DOS null, osteosarcoma cell line was transfected with DOS constructs and several independent clones exhibiting stable expression of epitope tagged DOS or DOS mutant(s) were identified and selected for further studies. As a control, the parental cell line 3081 was transfected with vector only.

For cell growth studies, formal cell counts were done. Five thousand cells from two independent clones, representative for each construct were plated in duplicate on 10 cm tissue culture plates. Cells were counted daily on a hemocytometer. Growth curves were plotted on a semi-log graph. DOS null, parental tumor cell line grew with rapid doubling time. Cell growth was significantly retarded with DOS+SSB reconstitution. The cell reconstituted with DOS+SSB P>L had an intermediate phenotype. These data suggest that reconstitution of DOS altered cell growth when plated on a plastic substrate.

These studies were extended to include growth on soft agar or anchorage independent growth. Several tumor cells exhibited anchorage independent growth which can be reverted by expression of tumor suppressor genes. Parental, DOS null osteosarcoma cells made large colonies as early as 10 days after suspension in agar. Expression of DOS in these cells inhibited growth on soft agar while expression of proline mutation had a partial phenotype. The cell growth data, both on plastic and soft agar support the hypothesis that DOS is a tumor suppressor gene and proline mutation in DOS appears to produce a loss of DOS function.

Stable reconstitution of the DOS null osteosarcoma cell line 3081 with DOS+SSB, changed the shape of the cells from spindle shaped fibroblast like cells to a polygonal epithelioid appearing cells. These cells are large, flat and show increased stress fibers when stained with phalloidin. On the contrary, cells expressing DOS+SSB P>L are small cells with a decrease in actin containing stress fiber staining. The morphology of DOS expressing cells when compared to parental cells did not show increase in lamellipodia which is a marker for increase in Rac activity and has been observed with expression of DOS homologue, DOCK180. The increase in stress fiber does suggest an increase in cellular Rho activity. Biochemical studies to elucidate the role of GTPase activation by DOS were carried out and are described below.

DOS Activates RAP 1

Small GTPases such as Rho, Rac and CDC42 play a critical role in diverse cellular functions as cell growth, cell movement and signal transduction. These proteins shuttle between an inactive GTP bound state and active GTP bound state. To determine positive and negative regulators of GTPase activation cascade, biochemical assays with in vitro binding selectively to the GTP bound state after cell lysis and comparing it to the levels of total (GTP and GDP bound form) have been established. Typically, these assays require co-transfection of putative activator with construct encoding GTPase in 293T cells. Point mutations in GTPases, which are dominant-negative and persistently active, respectively, were also used in the assay as positive and negative controls.

Both isoforms of DOS, when co-transfected with Rho, Rac and CDC 42 did not lead to GTPase activation. Other known members of CDM protein family, DOCK2 and DOCK180, are activators of Rac1. Our C elegans data showed that DOS is distinct from DOCK180 and presumably mediated its activity via another cellular GTPase. We turned our attention on Rap1 for the following reasons. As described above, all CDM protein family members bind adaptor protein Crk. The vast majority of Crk was bound to a protein called C3G which upon being tyrosine phosphorylation activates GTPase Rap1. There is little information available on Rap1 function. Since, Rap1 was cloned as a suppressor of Ras transformed cells, it was proposed that Rap1 is an antagonist of Ras and is anti-mitogenic. In Drosophila, Rap1 is extensively studied and is thought to play a critical role in cell-cell adhesion junctions.

Rap1 was co-transfected with DOS in 293T cell and lysates were bound to GST fusion protein of Ra1GDS-RBD domain which has a high affinity for GTP-Rap1. Total lysates were run on SDS-PAGE separately and ratio of active GTP-Rap1 to total cellular Rap1 was determined. Rap1 was activated by both isoforms of DOS, however, DOS P>L mutation was unable to do so. Persistantly active Rap, Rap63E, and dominant-negative mutant Rap, RapN17 were used as controls. Activation of Rap1 by DOS suggests that the putative tumor suppressor function of DOS may be mediated by Rap via inactivation of the mitogenic MAP kinase pathway and activation of cell-cell junctions.

DOS and Adherens Junctions

The DOS reconstituted osteosarcoma cells were used to study adherens junctions. These junctions are formed by the interaction of E-cadherin on neighboring cells. The intra-cellular tails of E-cadherin are bound to both a and b catenin. These junctions can be readily visualized using immuno-fluorescence when antibodies to beta-catenin are used. The DOS null osteosarcoma cell lines did not form adherens junctions and exhibited a complete loss of contact inhibition. The DOS reconstituted cells formed adherens junctions. There was no adherens junction formation and the cells appeared similar to the parental line when these osteoparcome cells were reconsitituted with DOS P>L. These observations validated the biochemical data on Rap1 activation by DOS and suggested that adherens junctions, a marker for Rap1 activity, can be rescued in a cell line that has lost DOS expression during tumorigenesis.

DOS Rescues Tumor Invasion

Although, suppression of growth on soft agar is indicative of tumor suppressive function, these assays are not always reliable. To address tumor formation in nude mice, subcutaneous injections of DOS null osteosarcoma 3081, 3081 reconstituted with either wild type or mutant DOS were performed. Mice were sacrificed 6 weeks after injection. DOS null and DOS mutant cell lines formed large tumors which invade surrounding fat, skin and muscle layers. DOS expressing tumors formed small, non-invasive, well-circumscribed tumors with surrounding muscle, skin and fat intact.

Our results suggest that DOS has tumor suppressor function. DOS is targeted by mutations in human cancers such as prostate, ovarian, colon and brain. These mutations are missense and are not polymorphisms, at least, the proline to leucine mutation seen in prostate and ovarian cancer cell lines. DOS suppresses growth in soft agar and suppresses tumor volume in nude mice. DOS activates Rap1, leading to formation of cell-cell junctions. Loss of DOS results in disruption of these junctions and these junctions can be rescued by reconstitution of DOS expression. It is likely that loss of adherens junctions lead to cell detachment and subsequent tumor invasion.

We are using RNA interference on DOS in normal cells and studying loss of adherens junction formation. Also, in osteosarcoma 3081 reconstituted with DOS, we are disrupting Rap activity and studying cell junctions and tumor formation in mice.

A mouse knockout model is also generated to assess DOS functional activities.

All references disclosed herein are incorporated by reference in their entirety.

### REFERENCES

1. 1 Haber, D. A. and Fearon, E. R. The promise of cancer genetics. Lancet 351 Suppl 2:SII1-8, 1998.
2. Loeb, K. R. and Loeb, L. A., Significance of multiple mutations in cancer. Carcinogenesis 21(3):379-85, 2000
3. Loeb, L. A., Springgate, C. F., Battula, N. Errors in DNA replication as a basis of malignant changes. Cancer Research 34 (9):2311-21, 1974
4. Haber D. and Harlow E. Tumor-suppressor genes: evolving definitions in the genomic age. Nature Genetics 16 (4):320-2, 1997
5. Fearon, E. R. Cancer progression. Current Biology 9 (23):R873-5, 1999
6. Fearon, E. R. and Dang, C. V. Cancer genetics: tumor suppressor meets oncogene. Current Biology 9 (2):R62-5, 1999
7. Fearon, E. R. Human cancer syndromes: clues to the origin and nature of cancer. Science 278:1043-50, 1997.
8. Kinzler, K. W. and Vogelstein B. Lessons from hereditary colorectal cancer. Cell. 87 (2):159-70, 1996
9. Gray, J. W. and Collins, C. Genome changes and gene expression in human solid tumors. Carcinogenesis. 21 (3):443-52, 2000.
10. Rowley, J. D. The critical role of chromosomal translocations in human leukemias. Annu. Rev. Genet. 32:495-519
11. Pinkel, D., Landegent, J., Collins, C., Fuscoe, J., Segraves, R., Lucas, J., Gray, J. Fluorescence in situ hybridization with human chromosome specific libraries: detection of trisomy 21 and translocations of chromosomes 4. Proc. Natl. Acad. Sci. 85 9138-9142, 1988
12. Speicher, M. R. and Ward D. C. The coloring of cytogenetics. Nature Med. 2:1046-1048 1996
13. Kallioniemi, O. P., Kallioniemi, A., Sudar, D., Rutovitz, D., Gray, J. W., Waldman, F., Pinkel, D. Comparative genomic hybridization: a rapid new method for detecting and mapping DNA amplification in tumors. Seminars in Cancer Biology. 4 (1):41-6, 1993
14. Lisitsyn, N., Lisitsyn, N., Wigler M. Cloning the differences between two complex genomes. Science. 259 (5097):946-51, 1993
15. Mollenhauer, J., Wiemann, S., Scheurlen, W., Korn, B., Hayashi, Y., Wilgenbus, K. K., von Deimling, A., Poustka A. DMBT1, a new member of the SRCR superfamily, on chromosome 10q25.3-26.1 is deleted in malignant brain tumours. Nature Genetics. 17 (1):32-9, 1997
16. Li, J., Yen, c., Liaw, D., Podsypanina, K., Bose, S., Wang, S., Puc, J., Miliaresis, C., Rodgers, L., McCombie, Bigner, S., Giovanella, B., Ittmann, M.,Tycko, B., Hibshoosh, H., wigler, M., Parsons, R. PTEN, a Putative Protein Tyrosine Phosphatase Gene Mutated in Human Brain, Breast, and Prostate Cancer. Science 275, 1943-1947, 1997
17. Schutte, M., D A Costa, L., Hahn, S., Moskaluk, C., Hoque, A., Rozenblum, E., Weinstein, C., Bittner, M., Meltzer, P., Trent, J., Yeo, C., Hruban, R., Kern, S. Identification by representational difference analysis of a homozygous deletion in pancreatic carcinoma that lies within the BRCA2 region. Proc. Natl. Acad. Sci. 92, 5950-5954, 1995
18. Lisitsyn N A. Representational difference analysis: finding the differences between genomes. Trends in Genetics 11 (8):303-7, 1995
19. Lisitsy, N. and Wigler, M. Representational difference analysis in detection of genetic lesions in cancer. Methods in Enzymology. 254:291-304, 1995.
20. Lisitsyn, N. A., Lisitsyn, N. M., Dalbagni, G., Barker, P., Sanchez, C. A., Gnarra, J. Linehan, W. M., Reid, B. J., Wigler, M. H. Comparative genomic analysis of tumors: detection of DNA losses and amplification. Proc. Natl. Acad. Sci. 92 (1):151-5, 1995
21. Baldocchi, R. A., Flaherty, L. Isolation of genomic fragments from polymorphic regions by representational difference analysis. Methods. 13 (4):337-46, 1997
22. Bonner, R. F., Emmert-Buck, M., Cole K., Pohida, T., Chuaqui, R., Goldstein, S., Liotta, L. A. Laser capture microdissection: molecular analysis of tissue. Science 78:1481-1483, 1997
23. Michiels, L., Van Leuven, F., van den Oord, J. J., De Wolf-Peeters, C., Delabie, J., Representational difference analysis using minute quantities of DNA. Nucleic Acids Research. 26 (15):3608-10, 1998
24. McClatchey, A. I. and Jacks, T. Tumor suppressor mutations in mice: the next Current Opinion in Genetics & Development. 8 (3):304-10, 1998
25. Balmain, A. Harris, C C. Carcinogenesis in mouse and human cells: parallels and paradoxes. Carcinogenesis. 21 (3):371-7, 2000
26. Balmain, A. and Nagase, H. Cancer resistance genes in mice: models for the study of tumor modifiers. Trends in Genetics. 14 (4):139-44, 1998
27. Bremner, R and Balmain, A. Genetic changes in skin tumors progression: correlation between presence of a mutant ras gene and loss of heterozygosity on mouse chromosome 7. Cell 61:407-417
28. Attardi, L. D. and Jacks T. The role of p53 in tumor suppression: lessons from mouse models. Cellular & Molecular Life Sciences. 55 (1):48-63, 1999
29. McClatchey, A. I., Saotome, I., Mercer, K., Crowley, D., Gusella, J. F., Bronson, R. T., Jacks, T. Mice heterozygous for a mutation at the Nf2 tumor suppressor locus develop a range of highly metastatic tumors. Genes & Development. 12 (8):1121-33, 1998
30. Battey, J., Jordan, E., Cox, D., Dove, W. An action plan for mouse genomics Nature Genet. 21:73-75, 1999.
31. Hasegawa, H., Kiyokawa, E., Tanaka, S., Nagashima, K., Gotoh, N., Shibuya, H., Kurata, T., Matsuda, M. Dock180, a major CRK-binding protein, alters cell morphology upon translocation to the cell membrane. Molecular and Cellular Biology, 16 1770-1776, 1996
32. Hall, A. Rho GTPases and the actin cytoskeleton. Science 279 509-514, 1998
33. Nolan, K., Barrett, K., Lu, Y., Hu. K., Vincent, S., Settleman, J. Myoblast city, the Drosophila homolog of DOCK180/CED-5, is required in a Rac signaling pathway utilized for multiple developmental processes. Genes and Development 12 3337-3342, 1998
34. Kumar C., Signaling by integrin receptors. Oncogene 17 1365-1373, 1998
35. Reddien, P., Horovitz, R CED-2/CrkII and CED-5/Rac control phagocytosis and cell migration in Cenorhabditis elegans. Nature Cell biology 2 131-135, 2000
36. Kiyokawa, E., Hashimoto, Y., Kobayashi, S., Sugimura, H., Kurata, T., Matsuda, M.

Activation of Rac1 by a Crk SH3-binding protein, DOCK180. Genes and Development 12 3331-3336, 1998
37. Klemke, R, Leng, J., Molander, R., Brooks., Vuori, K., Cheresh, D. Cas/Crk coupling serves as a molecular switch for induction of cell migration. J. Cell Biol. 140 961-972 1998
38. Cheresh, D., Leng, J., Klemke, R. Regulation of cell contraction and membrane ruffling by distinct signals in migratory cells. J. Cell Biol. 146 1107-1116 1999
39. Cho, S., Klemke, R Exracellular-regulated kinase activation and CAS/Crk coupling regulate cell migration and suppress apoptosis during invasion of the extracellular matrix. J. Cell Biol. 149 223-236 2000
40. Albert, M., Kim, J., Birge, R. αᵥβ₅ integrin recruits the CrkII-Dock180-Rac1 complex for phagocytosis of apoptotic cells. Nature Cell biology 2 899-905, 2000
41. Symons M., Settleman, J. Rho family GTPases: more than simple switches. Trends in Cell Biology 10 415-419 2000
42. Yu, Q., Stamenkovic, I. Cell surface-localized matrix metaloprotinase-9 proteolytically activates TGF-β and promotes tumor invasion and angiogenesis Genes and Development 14 163-176, 2000
43. Koike, M., Takeyuchi, S., Yokota, J., Hatta, Y., Miller, C., Tsuruoka, N, Koeffler, H. Frequent loss of heterozygosity in the region of the D7S523 locus in advanced ovarian cancer. Genes, Chromosomes and Cancer 19 1-5 1997
44. Okahashi, S., Shan, A., Ritland, S., Delacey, K., Botswick, D., Lieber, M., Thibodeau S., Jenkins, R. Frequent loss of heterozygosity at 7q31.1 in primary prostate cancer is associated with tumor aggressiveness and progression. Cancer Res. 55 4114-4119 1995
45. Shridhar V., Sun, Q., Miller O., Kalemkerian, G., Petros, J., Smith D. Loss of heterozygosity in the long arm of chromosome 7 in sporadic renal cell carcinomas. Oncogene 15 2727-2733 1997

1. An isolated nucleic acid molecule selected from the group consisting of:
(a) nucleic acid molecules which hybridize under stringent conditions to a nucleic acid molecule having a nucleotide sequence set forth as SEQ ID NO:1 or SEQ ID NO:3, and which code for a DOS protein,
(b) deletions, additions and substitutions of the nucleic acid molecules of (a),
(c) nucleic acid molecules that differ from the nucleic acid molecules of (a) or (b) in codon sequence due to the degeneracy of the genetic code, and
(d) complements of (a), (b) or (c).

2. The isolated nucleic acid molecule of claim 1, wherein the isolated nucleic acid molecule comprises SEQ ID NO:1.

3. The isolated nucleic acid molecule of claim 1, wherein the isolated nucleic acid molecule comprises SEQ ID NO:3.

4. An isolated nucleic acid molecule selected from the group consisting of:
(a) a unique fragment of the nucleotide sequence set forth as SEQ ID NO:1 or set forth as SEQ ID NO:3 between 12 and 115 nucleotides in length or more, and
(b) complements of (a),
wherein the unique fragments exclude nucleic acids having nucleotide sequences that are contained within SEQ ID NO:1 or SEQ ID NO:3, and that are known as of the filing date of this application.

5. The isolated nucleic acid molecule of claim 4 wherein the isolated nucleic acid molecule comprises SEQ ID NO: 31.

6. An isolated nucleic acid molecule selected from the group consisting of:
(a) nucleic acid molecules which hybridize under stringent conditions to a nucleic acid molecule having a nucleotide sequence selected from the group consisting of SEQ ID NOs:5, 7, 9, 11, 13, 15, 17, 19, 21, 23, 25, 27, or 29,
(b) deletions, additions and substitutions of the nucleic acid molecules of (a),
(c) nucleic acid molecules that differ from the nucleic acid molecules of (a) or (b) in codon sequence due to the degeneracy of the genetic code, and
(d) complements of (a), (b) or (c).

7. An expression vector comprising the isolated nucleic acid molecule of claim 1 operably linked to a promoter.

8. A host cell transformed or transfected with the expression vector of claim 7.

9. A transgenic non-human animal comprising the expression vector of claim 7.

10. A transgenic non-human animal which has reduced expression of a DOS nucleic acid molecule or of a Mutant DOS nucleic acid molecule.

11. An isolated protein encoded by the isolated nucleic acid molecule of claim 1.

12. The isolated protein of claim 11, wherein the isolated protein comprises of the amino acid sequence of selected from the group consisting of SEQ ID NOs: 2, 4, 6, 8, 10, 12, 14, 16, 18, 20, 22, 24, 26, 28, and 30.

13. The isolated protein of claim 11 wherein the isolated protein comprises SEQ ID NO: 32.

14. A binding polypeptide that selectively binds to the isolated protein of claim 11.

15-17. (canceled)

18. A composition comprising:
the nucleic acid of claim 1, and
a pharmaceutically acceptable carrier.

19. A composition comprising:
the protein encoded by the isolated nucleic acid molecule of claim 1, and
a pharmaceutically acceptable carrier.

20. A composition comprising:
the binding polypeptide of claim 14, and
a pharmaceutically acceptable carrier.

21. A method for making a medicament, comprising:
placing an active agent selected from the group consisting of: (a) the isolated nucleic acid molecules of claim 1, (b) the isolated protein of claim 11, and (c) the binding polypeptides of claim 14,
in a pharmaceutically acceptable carrier.

22. The method of claim 21, wherein placing comprises placing a therapeutically effective amount of the active agent in the pharmaceutically acceptable carrier to form one or more doses.

23. A method for diagnosing a disorder characterized by aberrant expression of a DOS molecule, comprising:
detecting in a first biological sample obtained from a subject, expression of a DOS molecule or a Mutant DOS molecule,
wherein decreased expression of a DOS molecule or the increased expression of a Mutant DOS molecule compared to a control sample indicates that the subject has a disorder characterized by aberrant expression of a DOS molecule.

24-25. (canceled)

26. The method of claim 23, wherein the disorder characterized by aberrant expression of a DOS molecule is selected from the group consisting of: a cancer, a tumor, a cytoskeleton disorder, and a cell migration disorder.

27-43. (canceled)

44. A kit for diagnosing a disorder associated with aberrant expression of a DOS molecule, comprising:
one or more nucleic acid molecules that hybridize to a DOS nucleic acid molecule or to a Mutant DOS nucleic acid molecule under stringent conditions,
one or more control agents, and
instructions for the use of the nucleic acid molecules, and agents in the diagnosis of a disorder associated with aberrant expression of a DOS molecule.

45-46. (canceled)

47. A kit for diagnosing a DOS tumor in a subject comprising:
one or more binding polypeptides that selectively bind to a DOS protein or a Mutant DOS protein,
one or more control agents, and
instructions for the use of the binding polypeptides, and agents in the diagnosis of a disorder associated with aberrant expression of a DOS molecule.

48-50. (canceled)

51. A method for treating a subject with a disorder characterized by aberrant expression of a DOS molecule, comprising
administering to the subject an effective amount of a DOS nucleic acid molecule to treat the disorder.

52. A method for treating a subject with a disorder characterized by aberrant expression of a DOS molecule, comprising
administering to the subject an effective amount of an anti-sense molecule to a Mutant DOS nucleic acid molecule to treat the disorder.

53. (canceled)

54. A method for treating a subject with a disorder characterized by aberrant expression of a DOS molecule, comprising
administering to the subject an effective amount of a DOS protein to treat the disorder.

55. A method for treating a subject with a disorder characterized by aberrant expression of a DOS molecule, comprising
administering to the subject an effective amount of a binding polypeptide to a Mutant DOS protein to treat the disorder.

56-58. (canceled)

59. A method for producing a DOS protein comprising
providing a DOS nucleic acid molecule operably linked to a promoter, wherein the DOS nucleic acid molecule encodes the DOS protein or a fragment thereof,
expressing the DOS nucleic acid molecule in an expression system, and
isolating the DOS protein or a fragment thereof from the expression system.

60. (canceled)

61. A method for producing a Mutant DOS protein comprising
providing a Mutant DOS nucleic acid molecule operably linked to a promoter, wherein the Mutant DOS nucleic acid molecule encodes the Mutant DOS protein or a fragment thereof,
expressing the Mutant DOS nucleic acid molecule in an expression system, and
isolating the Mutant DOS protein or a fragment thereof from the expression system.

62. (canceled)


SEQUENCE LISTING (32 sequences)

SEQ ID NO: 1 - DNA, 5901 bp, Homo sapiens
atgtggatacctacggagcacgagaaatacggcgtggttattgccagtttccgaggaaccgttccatatggcctgtcattggaaattggagatacagttcagatcctggagaagtgtgatggctggtacagaggatttgccttaaaaaacccaaatatcaagggtatatttccttccagctacgttcacttgaaaaatgcctgtgtaaagaacaaaggacaatttgaaatggttattcccactgaagactctgttatcacagaaatgacatcaacattaagagactggggaaccatgtggaaacaactctatgtgcgtaatgaaggcgatctcttccaccggctgtggcacatcatgaatgaaatcctggacctgaggcggcaggtgctggtgggccacctcacccacgaccggatgaaggacgtgaagcgccacattactgcccggcttgactggggcaatgaacaactgggactggacctggtgcctaggaaagagtacgcaatggtggatccggaagacatcagcattactgagctctaccgattgatggaacatcgacatcggaagaaagacaccccggtgcaggccagcagtcaccacctctttgtccagatgaagagcctcatgtgttccaacctgggagaggagctggaggtcatcttctcactctttgacagtaaagagaaccggccaatcagtgagagatttttcttgaggctgaatagaaacgggcttcccaaagcccctgataaaccggaacgacattgctccctctttgtggatttgggcagcagtgagctaagaaaggacatttatatcaccgtgcacattatccgaatcggtcgaatgggagcaggagaaaaaaagaatgcctgtagtgtccagtaccgacgaccctttggctgtgcagttcttagcatcgctgacctgctaacaggagagacaaaggatgacctcattctgaaagtatacatgtgtaacacagagagtgagtggtaccaaatccatgagaacatcatcaaaaagctgaatgcacgttataacttgactggctccaatgcaggtttagcagtttccttacagctattgcacggagacattgaacaaatcagaagggaatattcatcagtattttctcatggagtatccataacaaggaagctgggattttcaaatattattatgcctggtgaaatgaggaatgatttatatatcactattgaaaggggagaatttgagaaaggagggaagagcgtggccagaaatgtggaagttacgatgttcattgtagacagtagtggccaaaccctgaaggattttatctccttcggctctggggagccaccagccagtgagtaccactcctttgtgctttaccataacaacagtcccaggtggtctgaactgctgaaacttcccattcctgtggataaattccggggtgcacacatccgcttcgagtttcggcattgttccacaaaggagaaaggagagaagaagttgtttgggttttcttttgtccctctgatgcaagaagatggtaggactcttccagatggcactcatgagctcatcgtgcataagtgtgaagaaaacacaaatcttcaggatactacccgctacctcaaacttcccttttccaagggcattttccttgggaataataatcaagccatgaaggccacaaaggagtccttttgtattacatcttttctctgttccacaaaactcacacaaaatggtgatatgcttgatcttttgaaatggagaacccacccagacaagatcactggctgtctctctaaattaaaagaaattgatggctcagagatagtaaagtttctgcaggatacactggataccttatttggaattttagatgaaaattcccaaaaatatgggtctaaagtgtttgattctttggttcacataataaatttgctgcaagatagcaaatttcatcattttaaacctgtaatggacacttacattgagagtcattttgctggggcacttgcatacagagatctcatcaaagtgctcaaatggtacgtggaccggatcacagaagcagagcggcaagagcatatccaggaggtgctgaaggcacaagaatacatttttaagtatatagttcaatctcgaaggctgttttcccttgccactggtgggcaaaacgaagaggagttccgctgctgcattcaggagcttctcatgtcagtccgtttctttctttcgcaagagagcaaagggtctggagcattatctcagtcacaggctgtgtttctgagctctttccctgccgtgtactcagaactgttgaagctctttgatgtccgggaagtagccaacttggtccaggacaccctgggcagtctgccgaccatcctgcatgtggatgattccctgcaggccatcaaactgcagtgcattggcaaaaccgtggaaagccagctttataccaacccagattcccgatacattcttctgcctgtcgtgttacatcacctccacattcacttgcaagaacagaaggacctgatcatgtgtgcacgtatccttagcaacgtattttgtcttatcaagaaaaatagctcagaaaaatctgtgctggaggaaatagatgtgatagtggccagcttgctggatattctgctgaggaccatattggagatcaccagccgacctcagccatccagctcagcaatgcggttccagttccaggatgtcactggggagtttgttgcttgtctcctgtccctattacgacaaatgacagatagacattatcaacagcttcttgatagttttaatacaaaggaagaactaagggatttcctgctgcagatatttactgtgttccgaatattgatacgcccggagatgtttccaaaggactggactgttatgcgcttggttgctaacaatgttattattacaacagttctatacctctcagatgcacttcgtaagaacttcttaaatgaaaactttgattataagatctgggattcctacttttacctcgcagtcatttttataaaccagttgtgtctgcagttggagatgttcacaccttccaagaagaaaaaggtgttagaaaagtatggtgacatgcgggtaacaatgggttgtgaaattttcagcatgtggcaaaacctaggagagcacaagcttcattttatccctgccctgattggccccttcctagaagtgaccttgataccccagccaggtcttcggaatgtcatgattccaatttttcatgatatgatggactgggagcagaggcggagtggcaactttaaacaggtggaagccaagctaattgacaaactggatagcctgatgtcagaaggcaaaggtgacgaaacataccgcgagctcttcaacagtattctactaaagaaaattgagcgggaaacatggcgggaaagtggcgtttcattaattgctactgtaactcgtctaatggagaggttgttagattacagggactgcatgaaaatgggagaggtagatggcaaaaagattggctgcacagttagccttctgaacttctataagactgaactgaacaaggaggagatgtatatacgctacattcacaaactctatgatctgcatctcaaagcacagaactttacagaagctgcatataccctcctcttatatgacgagctactggaatggtctgatcggcccctcagggagttcctgacctaccccatgcaaacagaatggcagcgcaaagagcacctgcacctcaccatcatccagaactttgacagaggcaaatgttgggagaatggcattatcttgtgccggaagattgcagagcagtatgagagttattatgactacagaaacctgagcaagatgcggatgatggaagcctctttgtatgacaaaattatggaccagcaacgtcttgaaccagagttcttcagagttggattttatggaaaaaaatttccatttttcttaagaaataaggagtttgtgtgtcgagggcatgactacgagaggctggaagccttccaacagagaatgctgaacgagttcccccatgccatcgccatgcagcacgccaaccagcccgatgagaccatcttccaggcagaagctcagtatttgcagatatatgctgtgactcccattccagagagccaggaggtcctgcagagagagggtgttccggacaacatcaaaagcttctataaagtgaatcacatctggaaattccgctatgaccgaccatttcacaaaggcacaaaagataaagagaatgaattcaagagtctctgggtggagagaacgtcattatacttggtgcagagtttgcctggcatctctcgctggtttgaagtggaaaagcgtgaagtggtagaaatgagtcctctggaaaatgcaattgaagtgctagaaaataagaatcagcagctgaagactctgattagtcagtgtcagacaagacagatgcagaatattaatcccctgactatgtgcctgaatggagttatagatgctgcagttaatggtggcgtttccaggtatcaagaggcattctttgtcaaagaatatatcttaagtcaccctgaagatggggagaaaattgcacgattaagagagctgatgcttgagcaggcacagattctggaatttggtttggccgtgcatgagaagtttgtacctcaagatatgagaccccttcacaaaaagctggttgaccaattctttgtgatgaagtcgagcttagggatacaggagttctctgcttgtatgcaagccagtcctgtccattttcctaatggaagccctcgtgtgtgtagaaactcagcacctgcttctgtgagcccagatggtaccagggtaattcctagacgcagcccgttaagttacccagctgtcaaccgatattcttcctcctcactgtcctcacaagcttctgctgaagtaagcaatattacagggcaatcagaaagctctgatgaagtctttaacatgcagccaagtccatctacctcaagcttgagttctactcactcggcttcacctaatgtgacaagttctgctccatcgagtgccagagcttctcctttgttgtctgacaaacacaaacattcccgagaaaactcttgcctgtcaccaagagagagaccatgcagtgccatctatccaacacctgtggagccttcgcagaggatgctgtttaatcatattggagacggggccttgccacgcagtgacccaaatctctctgcacctgaaaaagctgtgaaccccacccctagcagctggagcctggacagtgggaaggaagccaagaacatgtcggatagtgggaaacttatctctccccctgtccctccaagacccacacagactgcttcaccagcaagacacacgacatcagtatccccctcgcctgccgggcgatctccattgaagggctctgtgcagtctttcaccccctctccagtggagtaccactcgccaggactcatctccaactcccctgtcttgtcgggcagctacagcagtgggatttcttctctcagccggtgcagcacgtcggaaacctcaggctttgaaaatcaggtgaatgaacagtcggcccccctgccggtgccagtgccggtgcccgtgccgagctacggcggggaggagccagtgcgcaaggagagcaagactccgcccccgtacagcgtctacgagcggactctgcggcgccccgtcccgctacctcacagcctctccatccccgtcacgtcggagccgcccgcgctgccccccaagcctctggcagcgcgatccagccacctggagaatggggcccggaggactgaccccggcccgcggcccaggcccctgccccgcaaggtctctcagttataa

SEQ ID NO: 2 - protein, 1966 aa, Homo sapiens
MWIPTEHEKYGVVIASFRGTVPYGLSLEIGDTVQILEKCDGWYRGFALKNPNIKGIFPSSYVHLKNACVKNKGQFEMVIPTEDSVITEMTSTLRDWGTMWKQLYVRNEGDLFHRLWHIMNEILDLRRQVLVGHLTHDRMKDVKRHITARLDWGNEQLGLDLVPRKEYAMVDPEDISITELYRLMEHRHRKKDTPVQASSHHLFVQMKSLMCSNLGEELEVIFSLFDSKENRPISERFFLRLNRNGLPKAPDKPERHCSLFVDLGSSELRKDIYITVHIIRIGRMGAGEKKNACSVQYRRPFGCAVLSIADLLTGETKDDLILKVYMCNTESEWYQIHENIIKKLNARYNLTGSNAGLAVSLQLLHGDIEQIRREYSSVFSHGVSITRKLGFSNIIMPGEMRNDLYITIERGEFEKGGKSVARNVEVTMFIVDSSGQTLKDFISFGSGEPPASEYHSFVLYHNNSPRWSELLKLPIPVDKFRGAHIRFEFRHCSTKEKGEKKLFGFSFVPLMQEDGRTLPDGTHELIVHKCEENTNLQDTTRYLKLPFSKGIFLGNNNQAMKATKESFCITSFLCSTKLTQNGDMLDLLKWRTHPDKITGCLSKLKEIDGSEIVKFLQDTLDTLFGILDENSQKYGSKVFDSLVHIINLLQDSKFHHFKPVMDTYIESHFAGALAYRDLIKVLKWYVDRITEAERQEHIQEVLKAQEYIFKYIVQSRRLFSLATGGQNEEEFRCCIQELLMSVRFFLSQESKGSGALSQSQAVFLSSFPAVYSELLKLFDVREVANLVQDTLGSLPTILHVDDSLQAIKLQCIGKTVESQLYTNPDSRYILLPVVLHHLHIHLQEQKDLIMCARILSNVFCLIKKNSSEKSVLEEIDVIVASLLDILLRTILEITSRPQPSSSAMRFQFQDVTGEFVACLLSLLRQMTDRHYQQLLDSFNTKEELRDFLLQIFTVFRILIRPEMFPKDWTVMRLVANNVIITTVLYLSDALRKNFLNENFDYKIWDSYFYLAVIFINQLCLQLEMFTPSKKKKVLEKYGDMRVTMGCEIFSMWQNLGEHKLHFIPALIGPFLEVTLIPQPGLRNVMIPIFHDMMDWEQRRSGNFKQVEAKLIDKLDSLMSEGKGDETYRELFNSILLKKIERETWRESGVSLIATVTRLMERLLDYRDCMKMGEVDGKKIGCTVSLLNFYKTELNKEEMYIRYIHKLYDLHLKAQNFTEAAYTLLLYDELLEWSDRPLREFLTYPMQTEWQRKEHLHLTIIQNFDRGKCWENGIILCRKIAEQYESYYDYRNLSKMRMMEASLYDKIMDQQRLEPEFFRVGFYGKKFPFFLRNKEFVCRGHDYERLEAFQQRMLNEFPHAIAMQHANQPDETIFQAEAQYLQIYAVTPIPESQEVLQREGVPDNIKSFYKVNHIWKFRYDRPFHKGTKDKENEFKSLWVERTSLYLVQSLPGISRWFEVEKREVVEMSPLENAIEVLENKNQQLKTLISQCQTRQMQNINPLTMCLNGVIDAAVNGGVSRYQEAFFVKEYILSHPEDGEKIARLRELMLEQAQILEFGLAVHEKFVPQDMRPLHKKLVDQFFVMKSSLGIQEFSACMQASPVHFPNGSPRVCRNSAPASVSPDGTRVIPRRSPLSYPAVNRYSSSSLSSQASAEVSNITGQSESSDEVFNMQPSPSTSSLSSTHSASPNVTSSAPSSARASPLLSDKHKHSRENSCLSPRERPCSAIYPTPVEPSQRMLFNHIGDGALPRSDPNLSAPEKAVNPTPSSWSLDSGKEAKNMSDSGKLISPPVPPRPTQTASPARHTTSVSPSPAGRSPLKGSVQSFTPSPVEYHSPGLISNSPVLSGSYSSGISSLSRCSTSETSGFENQVNEQSAPLPVPVPVPVPSYGGEEPVRKESKTPPPYSVYERTLRRPVPLPHSLSIPVTSEPPALPPKPLAARSSHLENGARRTDPGPRPRPLPRKVSQL

SEQ ID NO: 3 - DNA, 5898 bp, Mus musculus
atgtggatacctacggagcacgagaaatacggcgtggttattgccagctttcgaggcaccgtcccgtacggcctgtcattggagatcggagacactgttcagatcctggagaagtgcgacggctggtacagaggatttgccttaaaaaatcccaacatcaagggtatatttccttccagctatgttcacttgaaaaatgcctgtgtaaagaacaaaggacaatttgaaatggttattcccactgaagattctgttatcaccgagatgacatcaactttaagggactggggaaccatgtggaaacagctctatgtcagaaatgaaggggatctcttccatcgcctgaggcacatcatgaatgaaatcctcgatctgcgacggcaggtgctagttggccatctcacccatgacaggatgaaggatgtgaagcgccacattactgcccgactggattggggaaatgaacaactgggactagacctggtgcctaggaaggagtacgcaatggtagacccagaggacatcagcatcactgagctgtaccgactgatggaacatcgacatcggaagaaagacacccctgtgcaggccagcagtcaccacctctttgtccagatgaagagcctcatgtgttccaacctgggagaggagctagaggtcatcttctcactctttgacagcaaagagaatcgcccaatcagtgagagatttttcttgagactgaatagaaatgggcttcccaaagccccggataagcctgaaagacactgttctctctttgtggacttgggtagcagtgaactcaggaaggacatctacattaccgtgcacattatccgaattggtcgaatgggagcaggagagaaaaagaatgcctgcagtgttcagtaccggcgaccttttggctgtgcagtccttagcattgcagatctcctcaccggagagacaaaggatgacctggtcctgaaagtgtacatgtgtaatacagagagcgagtggtaccagatccacgagaacatcatcaagaagctgaatgcgcgctataatttgactggctccaatgcaggattagcagtttccctccaactgttacacggagacattgaacagatcagaagagaatactcctcagtgttctctcacggagtgtccataacgcggaagctgggtttttctgacattatcatgcctggtgaaatgaggaatgatttatatatcaccgtagaaaggggggaatttgagaaaggaggcaagagtgtggccagaaatgtggaagttacaatgttcattgtagacagtaatggtcagcccttaaaggattttatctcctttggctctggagagccgcctgctagtgagtaccactcctttgtgctctatcataacaacagtcccaggtggtcagagctgctgaaacttcctattcctgtggacaaattccggggctcccacatccgctttgagttccggcattgttccacaaaggagaaaggagaaaagaagttgttcggtttctctttcgtgcccctgatgcaagaagacggaagaactctcccagatggcacccacgagctcatcgtgcataagtgtgaagaaaacacaaaccttcaggatactacccgctacctcaagctcccattttccaaggtcatcttccttgggaacaataatcaaaccatgaaggccacaaaggaatccttttggataacatcatttctctgttccacaaaactcactcaaaatggtgatatgcttgatcttttgaaatggagaacccacccagacaagatcacaggctgtctctctaagttaaaagaaatcgatggctcagagatagtaaagtttctccaagacacactggataccttgtttggaatattagatgaaaattcccaaaaatatgggtctaaagtgtttgattctttggtccacataataaatttgctacaagatagcaagttccatcattttaagcctgtcatggacacttacattgagagtcactttgctggggcacttgcatacagagatctcatcaaagtgctcaagtggtacgtggacaggataacggaagcagagcgccaggagcatatccaggaagtgttgaaggcacaagagtacatttttaagtatatagttcaatcccgaaggctgttttcccttgccaccggagggcagaatgaagaggaattccgctgctgcatccaggagctccttatgtccgtccgcttcttcctctcacaagagagcaaagggactggagctttatctcagtcgcaggctgtgttcttgagctcctttccagctgtgtactcagaactgttgaagctctttgatgtccgggaagtggccaacttggtccaggacaccctgggcagcctacctaccatcatgcatgtggacgattctctgcaggcaattaagcttcagtgcattggcaaaactgtcgagagccagctctataccaacccagactcgcggtacattctcctgcctgtcgtactgcatcacctccacatccacttgcaagaacagaaggacttgatcatgtgtgcacgtatccttagcaacgtgttctgtctcatcaagaaaaatagctcagaaaagtctgtgctggaggaaatagacgtgatagtggccagcttgctggatatcctgctcaggaccatattggagatcactagtcggcctcaagcatccagctccgccatgaggctgcagttccaggatgtcactggggaatttgttgcctgtctcctttccctattacgacaaatgacagatagacattatcagcaacttctgaatagtttcagtactaaagaggagctgagggatttcttgctgcagatttttaccgttttccgaatattgatacgcccagagatgtttcccaaggactggactgtcatgcgcctggttgctaacaatgttatcatcacaacagttctgtacctctccgatgcactccgtaagaacttcttaaatgaaaactttgattataagatctgggattcctacttttacctcgctgtcatttttataaaccagttgtgtctgcagctggagatgttcacaccttctaaaaagaaaaaggtgctagaaaagtatggtgatatgcgcgtcaccatgggttgtgaaatttttagcatgtggcaaaacctaggagaacacaaacttcatttcatccctgccctgattgggccctttctggaagtaaccttgataccccagccagatctccggaatgtcatgattcctatttttcacgatatgatggactgggagcaaagaaggagtggcaactttaaacaggtggaggccaagctaattgacaaattggacagcctgatgtcggagggcaaaggtgatgaaacgtaccgtgagctcttcaacagtattctactaaagaaaattgagcgggaaacatggagggaaagtggcgtttcattaattgctactgtaactcggctgatggagaggttgttagactacagggactgcatgaagataggagaggtggatggcaaaaagattggctgcacagttagccttttgaacttctataagacggagctgaacaaggaggagatgtatatccgctacatccacaaactctacgatctgcatctcaaggcgcagaacttcacagaggctgcctacactctcctgttgtatgacgagctgctggagtggtctgatcggcccctcagggagttcctgacctaccccatgcagaccgaatggcagcgcaaggagcacctgcacctcaccatcatccagaacttcgaccgaggcaaatgttgggagaatggcattatcttgtgccggaagattgcagagcaatatgagagttactatgactacaggaacctgagcaagatgaggatgatggaagcctccttgtatgacaaaatcatggaccagcagcgcctggaacctgaattcttcagagttggattttatgggaaaaagtttccatttttcttaagaaataaggagtttgtatgtcgagggcatgactatgaaagactggaggccttccaacagcgaatgctgaacgagttcccccatgccatcgccatgcagcatgccaaccagcccgacgagaccatcttccaggcagaggctcagtatttacagatatatgctgtgactcccattccagagagccaggaggtactgcagagagagggggtcccagacaacatcaaaagcttctataaagtgaaccacatttggaagttccgctacgacaggccattccacaaaggcgcaaaagacaaggagaacgaattcaagagtctctgggtggaaaggacatcgctgtacttggtccagagcttacccggcatctcccgctggttcgaagtggaaaagcgagaagtggtagagatgagtcctctggaaaacgccatcgaagtgttagaaaacaagaaccagcagctgaagaccctgatcagccagtgtcagacgaggcagatgcagaatatcaaccccttgaccatgtgcctcaatggagtcatagatgctgcagtgaacggtggggtctccaggtatcaagaggcattctttgtcaaagactatattttaagtcatcctgaagatggggagaagattgcacggttaagagaactgatgcttgagcaggcacagattctggaatttgggctggccgtgcacgagaagtttgtacctcaagatatgagaccccttcacaaaaaactggtggaccagttctttgtgatgaaatcgagctttgggatacaggagttccctgcttgtatccaggccagccctgtccattttcccaatggaagccctcgtgtgtgtagaaactcagcccctgcttccatgagtccagatggtaccagggtaatccctagacgcagcccattaagttacccagctgtcaaccggtattcctcgtcctcgctgtcctcacaggcctctgctgaagtaagcaatattacagggcaatcagaaagctctgatgaagtctttaacatgcagcccagtccgtctacctcaagcctgagttccactcactctgcttcacctaatgtgaccagttctgctccgtcgagtgccagagcttcgcctttgttgtctgacaaacacaaacattccagagaaaactcttgcctgtccccaagagatagaccgtgcagtgccatttacccaacacccgtggagccttcgcagaggatgctgtttaatcatattggagatggggccttgcctcgaagtgaccccaacctttctgcacctgaaagagctgtgaatcccacccctagcagctggagcctggacagtgggaaagaagccaagaacatgtcggacagtgggaaacttatctctccccctgtccctccaaggcccacacagactgcttcgccagcaagacacaccacatcagtgtccccctcgcctgctggacgttctccattgaagggctccgtacagtccttcaccccatctccagtggagtacaactccccaggactgagttcaaattccccagtcttgtccggcagctacagcagtgggatttcctcgcttagccggtgtagcacgtcggaaacctcgggctttgaaaaccaagccaatgaacagtcagtgcctgtgccggtgccggtgcccgtgcccagcttcagcgggtcggaggaacctgtacgcaaggagagcaaaaccccacccccctacagtgtctacgagcggactctgcggcgccctgtcccgctacctcacagcctctccatccctgtcacctctgagccacctgctctgccccccaagcccttggctgcgcggtccagccacctggagaatgggacccgtaggactgagcctggccctaggcccaggcccctgccccgtaaggtgtctcagctataa

SEQ ID NO: 4 - protein, 1965 aa, Mus musculus
MWIPTEHEKYGVVIASFRGTVPYGLSLEIGDTVQILEKCDGWYRGFALKNPNIKGIFPSSYVHLKNACVKNKGQFEMVIPTEDSVITEMTSTLRDWGTMWKQLYVRNEGDLFHRLRHIMNEILDLRRQVLVGHLTHDRMKDVKRHITARLDWGNEQLGLDLVPRKEYAMVDPEDISITELYRLMEHRHRKKDTPVQASSHHLFVQMKSLMCSNLGEELEVIFSLFDSKENRPISERFFLRLNRNGLPKAPDKPERHCSLFVDLGSSELRKDIYITVHIIRIGRMGAGEKKNACSVQYRRPFGCAVLSIADLLTGETKDDLVLKVYMCNTESEWYQIHENIIKKLNARYNLTGSNAGLAVSLQLLHGDIEQIRREYSSVFSHGVSITRKLGFSDIIMPGEMRNDLYITVERGEFEKGGKSVARNVEVTMFIVDSNGQPLKDFISFGSGEPPASEYHSFVLYHNNSPRWSELLKLPIPVDKFRGSHIRFEFRHCSTKEKGEKKLFGFSFVPLMQEDGRTLPDGTHELIVHKCEENTNLQDTTRYLKLPFSKVIFLGNNNQTMKATKESFWITSFLCSTKLTQNGDMLDLLKWRTHPDKITGCLSKLKEIDGSEIVKFLQDTLDTLFGILDENSQKYGSKVFDSLVHIINLLQDSKFHHFKPVMDTYIESHFAGALAYRDLIKVLKWYVDRITEAERQEHIQEVLKAQEYIFKYIVQSRRLFSLATGGQNEEEFRCCIQELLMSVRFFLSQESKGTGALSQSQAVFLSSFPAVYSELLKLFDVREVANLVQDTLGSLPTIMHVDDSLQAIKLQCIGKTVESQLYTNPDSRYILLPVVLHHLHIHLQEQKDLIMCARILSNVFCLIKKNSSEKSVLEEIDVIVASLLDILLRTILEITSRPQASSSAMRLQFQDVTGEFVACLLSLLRQMTDRHYQQLLNSFSTKEELRDFLLQIFTVFRILIRPEMFPKDWTVMRLVANNVIITTVLYLSDALRKNFLNENFDYKIWDSYFYLAVIFINQLCLQLEMFTPSKKKKVLEKYGDMRVTMGCEIFSMWQNLGEHKLHFIPALIGPFLEVTLIPQPDLRNVMIPIFHDMMDWEQRRSGNFKQVEAKLIDKLDSLMSEGKGDETYRELFNSILLKKIERETWRESGVSLIATVTRLMERLLDYRDCMKIGEVDGKKIGCTVSLLNFYKTELNKEEMYIRYIHKLYDLHLKAQNFTEAAYTLLLYDELLEWSDRPLREFLTYPMQTEWQRKEHLHLTIIQNFDRGKCWENGIILCRKIAEQYESYYDYRNLSKMRMMEASLYDKIMDQQRLEPEFFRVGFYGKKFPFFLRNKEFVCRGHDYERLEAFQQRMLNEFPHAIAMQHANQPDETIFQAEAQYLQIYAVTPIPESQEVLQREGVPDNIKSFYKVNHIWKFRYDRPFHKGAKDKENEFKSLWVERTSLYLVQSLPGISRWFEVEKREVVEMSPLENAIEVLENKNQQLKTLISQCQTRQMQNINPLTMCLNGVIDAAVNGGVSRYQEAFFVKDYILSHPEDGEKIARLRELMLEQAQILEFGLAVHEKFVPQDMRPLHKKLVDQFFVMKSSFGIQEFPACIQASPVHFPNGSPRVCRNSAPASMSPDGTRVIPRRSPLSYPAVNRYSSSSLSSQASAEVSNITGQSESSDEVFNMQPSPSTSSLSSTHSASPNVTSSAPSSARASPLLSDKHKHSRENSCLSPRDRPCSAIYPTPVEPSQRMLFNHIGDGALPRSDPNLSAPERAVNPTPSSWSLDSGKEAKNMSDSGKLISPPVPPRPTQTASPARHTTSVSPSPAGRSPLKGSVQSFTPSPVEYNSPGLSSNSPVLSGSYSSGISSLSRCSTSETSGFENQANEQSVPVPVPVPVPSFSGSEEPVRKESKTPPPYSVYERTLRRPVPLPHSLSIPVTSEPPALPPKPLAARSSHLENGTRRTEPGPRPRPLPRKVSQL

SEQ ID NO: 5 - DNA, 5901 bp, Homo sapiens
atgtggatacctacggagcacgagaaatacggcgtggttattgccagtttccgaggaaccgttccatatggcctgtcattggaaattggagatacagttcagatcctggagaagtgtgatggctggtacagaggatttgccttaaaaaacccaaatatcaagggtatatttccttccagctacgttcacttgaaaaatgcctgtgtaaagaacaaaggacaatttgaaatggttattcccactgaagactctgttatcacagaaatgacatcaacattaagagactggggaaccatgtggaaacaactctatgtgcgtaatgaaggcgatctcttccaccggctgtggcacatcatgaatgaaatcctggacctgaggcggcaggtgctggtgggccacctcacccacgaccggatgaaggacgtgaagcgccacattactgcccggcttgactggggcaatgaacaactgggactggacctggtgcctaggaaagagtacgcaatggtggatccggaagacatcagcattactgagctctaccgattgatggaacatcgacatcggaagaaagacaccccggtgcaggccagcagtcaccacctctttgtccagatgaagagcctcatgtgttccaacctgggagaggagctggaggtcatcttctcactctttgacagtaaagagaaccggccaatcagtgagagatttttcttgaggctgaatagaaacgggcttcccaaagcccctgataaaccggaacgacattgctccctctttgtggatttgggcagcagtgagctaagaaaggacatttatatcaccgtgcacattatccgaatcggtcgaatgggagcaggagaaaaaaagaatgcctgtagtgtccagtaccgacgaccctttggctgtgcagttcttagcatcgctgacctgctaacaggagagacaaaggatgacctcattctgaaagtatacatgtgtaacacagagagtgagtggtaccaaatccatgagaacatcatcaaaaagctgaatgcacgttataacttgactggctccaatgcaggtttagcagtttccttacagctattgcacggagacattgaacaaatcagaagggaatattcatcagtattttctcatggagtatccataacaaggaagctgggattttcaaatattattatgcctggtgaaatgaggaatgatttatatatcactattgaaaggggagaatttgagaaaggagggaagagcgtggccagaaatgtggaagttacgatgttcattgtagacagtagtggccaaaccctgaaggattttatctccttcggctctggggagccaccagccagtgagtaccactcctttgtgctttaccataacaacagtcccaggtggtctgaactgctgaaacttcccattcctgtggataaattccggggtgcacacatccgcttcgagtttcggcattgttccacaaaggagaaaggagagaagaagttgtttgggttttcttttgtccctctgatgcaagaagatggtaggactcttccagatggcactcatgagctcatcgtgcataagtgtgaagaaaacacaaatcttcaggatactacccgctacctcaaacttcccttttccaagggcattttccttgggaataataatcaagccatgaaggccacaaaggagtccttttgtattacatcttttctctgttccacaaaactcacacaaaatggtgatatgcttgatcttttgaaatggagaacccacccagacaagatcactggctgtctctctaaattaaaagaaattgatggctcagagatagtaaagtttctgcaggatacactggataccttatttggaattttagatgaaaattcccaaaaatatgggtctaaagtgtttgattctttggttcacataataaatttgctgcaagatagcaaatttcatcattttaaacctgtaatggacacttacattgagagtcattttgctggggcacttgcatacagagatctcatcaaagtgctcaaatggtacgtggaccggatcacagaagcagagcggcaagagcatatccaggaggtgctgaaggcacaagaatacatttttaagtatatagttcaatctcgaaggctgttttcccttgccactggtgggcaaaacgaagaggagttccgctgctgcattcaggagcttctcatgtcagtccgtttctttctttcgcaagagagcaaagggtctggagcattatctcagtcacaggctgtgtttctgagctctttccctgccgtgtactcagaactgttgaagctctttgatgtccgggaagtagccaacttggtccaggacaccctgggcagtctgccgaccatcctgcatgtggatgattccctgcaggccatcaaactgcagtgcattggcaaaaccgtggaaagccagctttataccaacccagattcccgatacattcttctgcctgtcgtgttacatcacctccacattcacttgcaagaacagaaggacctgatcatgtgtgcacgtatccttagcaacgtattttgtcttatcaagaaaaatagctcagaaaaatctgtgctggaggaaatagatgtgatagtggccagcttgctggatattctgctgaggaccatattggagatcaccagccgacctcagccatccagctcagcaatgcggttccagttccaggatgtcactggggagtttgttgcttgtctcctgtccctattacgacaaatgacagatagacattatcaacagcttcttgatagttttaatacaaaggaagaactaagggatttcctgctgcagatatttactgtgttccgaatattgatacgcccggagatgtttccaaaggactggactgttatgcgcttggttgctaacaatgttattattacaacagttctatacctctcagatgcacttcgtaagaacttcttaaatgaaaactttgattataagatctgggattcctacttttacctcgcagtcatttttataaaccagttgtgtctgcagttggagatgttcacaccttccaagaagaaaaaggtgttagaaaagtatggtgacatgcgggtaacaatgggttgtgaaattttcagcatgtggcaaaacctaggagagcacaagcttcattttatccctgccctgattggccccttcctagaagtgaccttgataccccagccaggtcttcggaatgtcatgattccaatttttcatgatatgatggactgggagcagaggcggagtggcaactttaaacaggtggaagccaagctaattgacaaactggatagcctgatgtcagaaggcaaaggtgacgaaacataccgcgagctcttcaacagtattctactaaagaaaattgagcgggaaacatggcgggaaagtggcgtttcattaattgctactgtaactcgtctaatggagaggttgttagattacagggactgcatgaaaatgggagaggtagatggcaaaaagattggctgcacagttagccttctgaacttctataagactgaactgaacaaggaggagatgtatatacgctacattcacaaactctatgatctgcatctcaaagcacagaactttacagaagctgcatataccctcctcttatatgacgagctactggaatggtctgatcggcccctcagggagttcctgacctaccccatgcaaacagaatggcagcgcaaagagcacctgcacctcaccatcatccagaactttgacagaggcaaatgttgggagaatggcattatcttgtgccggaagattgcagagcagtatgagagttattatgactacagaaacctgagcaagatgcggatgatggaagcctctttgtatgacaaaattatggaccagcaacgtcttgaaccagagttcttcagagttggattttatggaaaaaaatttccatttttcttaagaaataaggagtttgtgtgtcgagggcatgactacgagaggctggaagccttccaacagagaatgctgaacgagttcccccatgccatcgccatgcagcacgccaaccagcccgatgagaccatcttccaggcagaagctcagtatttgcagatatatgctgtgactcccattccagagagccaggaggtcctgcagagagagggtgttccggacaacatcaaaagcttctataaagtgaatcacatctggaaattccgctatgaccgaccatttcacaaaggcacaaaagataaagagaatgaattcaagagtctctgggtggagagaacgtcattatacttggtgcagagtttgcctggcatctctcgctggtttgaagtggaaaagcgtgaagtggtagaaatgagtcctctggaaaatgcaattgaagtgctagaaaataagaatcagcagctgaagactctgattagtcagtgtcagacaagacagatgcagaatattaatcccctgactatgtgcctgaatggagttatagatgctgcagttaatggtggcgtttccaggtatcaagaggcattctttgtcaaagaatatatcttaagtcaccctgaagatggggagaaaattgcacgattaagagagctgatgcttgagcaggcacagattctggaatttggtttggccgtgcatgagaagtttgtacctcaagatatgagaccccttcacaaaaagctggttgaccaattctttgtgatgaagtcgagcttagggatacaggagttctctgcttgtatgcaagccagtcctgtccattttcctaatggaagccctcgtgtgtgtagaaactcagcacctgcttctgtgagcccagatggtaccagggtaattcctagacgcagcccgttaagttacccagctgtcaaccgatattcttcctcctcactgtcctcacaagcttctgctgaagtaagcaatattacagggcaatcagaaagctctgatgaagtctttaacatgcagccaagtccatctacctcaagcttgagttctactcactcggcttcacctaatgtgacaagttctgctccatcgagtgccagagcttctcctttgttgtctgacaaacacaaacattcccgagaaaactcttgcctgtcaccaagagagagaccatgcagtgccatctatccaacacctgtggagccttcgcagaggatgctgtttaatcatattggagacggggccttgccacgcagtgacccaaatctctctgcacctgaaaaagctgtgaaccccacccctagcagctggagcctggacagtgggaaggaagccaagaacatgtcggatagtgggaaacttatctctccccctgtccctccaagacccacacagactgcttcaccagcaagacacacgacatcagtatccccctcgcctgccgggcgatctccattgaagggctctgtgcagtctttcaccccctctccagtggagtaccactcgccaggactcatctccaactcccctgtcttgtcgggcagctacagcagtgggatttcttctctcagccggtgcagcacgtcggaaacctcaggctttgaaaatcaggtgaatgaacagtcggcccccctgccggtgccagtgccggtgcccatgccgagctacggcggggaggagccagtgcgcaaggagagcaagactccgcccccgtacagcgtctacgagcggactctgcggcgccccgtcccgctacctcacagcctctccatccccgtcacgtcggagccgcccgcgctgccccccaagcctctggcagcgcgatccagccacctggagaatggggcccggaggactgaccccggcccgcggcccaggcccctgccccgcaaggtctctcagttataa

SEQ ID NO: 6 - protein, 1966 aa, Homo sapiens
MWIPTEHEKYGVVIASFRGTVPYGLSLEIGDTVQILEKCDGWYRGFALKNPNIKGIFPSSYVHLKNACVKNKGQFEMVIPTEDSVITEMTSTLRDWGTMWKQLYVRNEGDLFHRLWHIMNEILDLRRQVLVGHLTHDRMKDVKRHITARLDWGNEQLGLDLVPRKEYAMVDPEDISITELYRLMEHRHRKKDTPVQASSHHLFVQMKSLMCSNLGEELEVIFSLFDSKENRPISERFFLRLNRNGLPKAPDKPERHCSLFVDLGSSELRKDIYITVHIIRIGRMGAGEKKNACSVQYRRPFGCAVLSIADLLTGETKDDLILKVYMCNTESEWYQIHENIIKKLNARYNLTGSNAGLAVSLQLLHGDIEQIRREYSSVFSHGVSITRKLGFSNIIMPGEMRNDLYITIERGEFEKGGKSVARNVEVTMFIVDSSGQTLKDFISFGSGEPPASEYHSFVLYHNNSPRWSELLKLPIPVDKFRGAHIRFEFRHCSTKEKGEKKLFGFSFVPLMQEDGRTLPDGTHELIVHKCEENTNLQDTTRYLKLPFSKGIFLGNNNQAMKATKESFCITSFLCSTKLTQNGDMLDLLKWRTHPDKITGCLSKLKEIDGSEIVKFLQDTLDTLFGILDENSQKYGSKVFDSLVHIINLLQDSKFHHFKPVMDTYIESHFAGALAYRDLIKVLKWYVDRITEAERQEHIQEVLKAQEYIFKYIVQSRRLFSLATGGQNEEEFRCCIQELLMSVRFFLSQESKGSGALSQSQAVFLSSFPAVYSELLKLFDVREVANLVQDTLGSLPTILHVDDSLQAIKLQCIGKTVESQLYTNPDSRYILLPVVLHHLHIHLQEQKDLIMCARILSNVFCLIKKNSSEKSVLEEIDVIVASLLDILLRTILEITSRPQPSSSAMRFQFQDVTGEFVACLLSLLRQMTDRHYQQLLDSFNTKEELRDFLLQIFTVFRILIRPEMFPKDWTVMRLVANNVIITTVLYLSDALRKNFLNENFDYKIWDSYFYLAVIFINQLCLQLEMFTPSKKKKVLEKYGDMRVTMGCEIFSMWQNLGEHKLHFIPALIGPFLEVTLIPQPGLRNVMIPIFHDMMDWEQRRSGNFKQVEAKLIDKLDSLMSEGKGDETYRELFNSILLKKIERETWRESGVSLIATVTRLMERLLDYRDCMKMGEVDGKKIGCTVSLLNFYKTELNKEEMYIRYIHKLYDLHLKAQNFTEAAYTLLLYDELLEWSDRPLREFLTYPMQTEWQRKEHLHLTIIQNFDRGKCWENGIILCRKIAEQYESYYDYRNLSKMRMMEASLYDKIMDQQRLEPEFFRVGFYGKKFPFFLRNKEFVCRGHDYERLEAFQQRMLNEFPHAIAMQHANQPDETIFQAEAQYLQIYAVTPIPESQEVLQREGVPDNIKSFYKVNHIWKFRYDRPFHKGTKDKENEFKSLWVERTSLYLVQSLPGISRWFEVEKREVVEMSPLENAIEVLENKNQQLKTLISQCQTRQMQNINPLTMCLNGVIDAAVNGGVSRYQEAFFVKEYILSHPEDGEKIARLRELMLEQAQILEFGLAVHEKFVPQDMRPLHKKLVDQFFVMKSSLGIQEFSACMQASPVHFPNGSPRVCRNSAPASVSPDGTRVIPRRSPLSYPAVNRYSSSSLSSQASAEVSNITGQSESSDEVFNMQPSPSTSSLSSTHSASPNVTSSAPSSARASPLLSDKHKHSRENSCLSPRERPCSAIYPTPVEPSQRMLFNHIGDGALPRSDPNLSAPEKAVNPTPSSWSLDSGKEAKNMSDSGKLISPPVPPRPTQTASPARHTTSVSPSPAGRSPLKGSVQSFTPSPVEYHSPGLISNSPVLSGSYSSGISSLSRCSTSETSGFENQVNEQSAPLPVPVPVPMPSYGGEEPVRKESKTPPPYSVYERTLRRPVPLPHSLSIPVTSEPPALPPKPLAARSSHLENGARRTDPGPRPRPLPRKVSQL

SEQ ID NO: 7 - DNA, 5901 bp, Homo sapiens; identical to SEQ ID NO: 5

SEQ ID NO: 8 - protein, 1966 aa, Homo sapiens; identical to SEQ ID NO: 6

SEQ ID NO: 9 - DNA, 5901 bp, Homo sapiens
atgtggatacctacggagcacgagaaatacggcgtggttattgccagtttccgaggaaccgttccatatggcctgtcattggaaattggagatacagttcagatcctggagaagtgtgatggctggtacagaggatttgccttaaaaaacccaaatatcaagggtatatttccttccagctacgttcacttgaaaaatgcctgtgtaaagaacaaaggacaatttgaaatggttattcccactgaagactctgttatcatagaaatgacatcaacattaagagactggggaaccatgtggaaacaactctatgtgcgtaatgaaggcgatctcttccaccggctgtggcacatcatgaatgaaatcctggacctgaggcggcaggtgctggtgggccacctcacccacgaccggatgaaggacgtgaagcgccacattactgcccggcttgactggggcaatgaacaactgggactggacctggtgcctaggaaagagtacgcaatggtggatccggaagacatcagcattactgagctctaccgattgatggaacatcgacatcggaagaaagacaccccggtgcaggccagcagtcaccacctctttgtccagatgaagagcctcatgtgttccaacctgggagaggagctggaggtcatcttctcactctttgacagtaaagagaaccggccaatcagtgagagatttttcttgaggctgaatagaaacgggcttcccaaagcccctgataaaccggaacgacattgctccctctttgtggatttgggcagcagtgagctaagaaaggacatttatatcaccgtgcacattatccgaatcggtcgaatgggagcaggagaaaaaaagaatgcctgtagtgtccagtaccgacgaccctttggctgtgcagttcttagcatcgctgacctgctaacaggagagacaaaggatgacctcattctgaaagtatacatgtgtaacacagagagtgagtggtaccaaatccatgagaacatcatcaaaaagctgaatgcacgttataacttgactggctccaatgcaggtttagcagtttccttacagctattgcacggagacattgaacaaatcagaagggaatattcatcagtattttctcatggagtatccataacaaggaagctgggattttcaaatattattatgcctggtgaaatgaggaatgatttatatatcactattgaaaggggagaatttgagaaaggagggaagagcgtggccagaaatgtggaagttacgatgttcattgtagacagtagtggccaaaccctgaaggattttatctccttcggctctggggagccaccagccagtgagtaccactcctttgtgctttaccataacaacagtcccaggtggtctgaactgctgaaacttcccattcctgtggataaattccggggtgcacacatccgcttcgagtttcggcattgttccacaaaggagaaaggagagaagaagttgtttgggttttcttttgtccctctgatgcaagaagatggtaggactcttccagatggcactcatgagctcatcgtgcataagtgtgaagaaaacacaaatcttcaggatactacccgctacctcaaacttcccttttccaagggcattttccttgggaataataatcaagccatgaaggccacaaaggagtccttttgtattacatcttttctctgttccacaaaactcacacaaaatggtgatatgcttgatcttttgaaatggagaacccacccagacaagatcactggctgtctctctaaattaaaagaaattgatggctcagagatagtaaagtttctgcaggatacactggataccttatttggaattttagatgaaaattcccaaaaatatgggtctaaagtgtttgattctttggttcacataataaatttgctgcaagatagcaaatttcatcattttaaacctgtaatggacacttacattgagagtcattttgctggggcacttgcatacagagatctcatcaaagtgctcaaatggtacgtggaccggatcacagaagcagagcggcaagagcatatccaggaggtgctgaaggcacaagaatacatttttaagtatatagttcaatctcgaaggctgttttcccttgccactggtgggcaaaacgaagaggagttccgctgctgcattcaggagcttctcatgtcagtccgtttctttctttcgcaagagagcaaagggtctggagcattatctcagtcacaggctgtgtttctgagctctttccctgccgtgtactcagaactgttgaagctctttgatgtccgggaagtagccaacttggtccaggacaccctgggcagtctgccgaccatcctgcatgtggatgattccctgcaggccatcaaactgcagtgcattggcaaaaccgtggaaagccagctttataccaacccagattcccgatacattcttctgcctgtcgtgttacatcacctccacattcacttgcaagaacagaaggacctgatcatgtgtgcacgtatccttagcaacgtattttgtcttatcaagaaaaatagctcagaaaaatctgtgctggaggaaatagatgtgatagtggccagcttgctggatattctgctgaggaccatattggagatcaccagccgacctcagccatccagctcagcaatgcggttccagttccaggatgtcactggggagtttgttgcttgtctcctgtccctattacgacaaatgacagatagacattatcaacagcttcttgatagttttaatacaaaggaagaactaagggatttcctgctgcagatatttactgtgttccgaatattgatacgcccggagatgtttccaaaggactggactgttatgcgcttggttgctaacaatgttattattacaacagttctatacctctcagatgcacttcgtaagaacttcttaaatgaaaactttgattataagatctgggattcctacttttacctcgcagtcatttttataaaccagttgtgtctgcagttggagatgttcacaccttccaagaagaaaaaggtgttagaaaagtatggtgacatgcgggtaacaatgggttgtgaaattttcagcatgtggcaaaacctaggagagcacaagcttcattttatccctgccctgattggccccttcctagaagtgaccttgataccccagccaggtcttcggaatgtcatgattccaatttttcatgatatgatggactgggagcagaggcggagtggcaactttaaacaggtggaagccaagctaattgacaaactggatagcctgatgtcagaaggcaaaggtgacgaaacataccgcgagctcttcaacagtattctactaaagaaaattgagcgggaaacatggcgggaaagtggcgtttcattaattgctactgtaactcgtctaatggagaggttgttagattacagggactgcatgaaaatgggagaggtagatggcaaaaagattggctgcacagttagccttctgaacttctataagactgaactgaacaaggaggagatgtatatacgctacattcacaaactctatgatctgcatctcaaagcacagaactttacagaagctgcatataccctcctcttatatgacgagctactggaatggtctgatcggcccctcagggagttcctgacctaccccatgcaaacagaatggcagcgcaaagagcacctgcacctcaccatcatccagaactttgacagaggcaaatgttgggagaatggcattatcttgtgccggaagattgcagagcagtatgagagttattatgactacagaaacctgagcaagatgcggatgatggaagcctctttgtatgacaaaattatggaccagcaacgtcttgaaccagagttcttcagagttggattttatggaaaaaaatttccatttttcttaagaaataaggagtttgtgtgtcgagggcatgactacgagaggctggaagccttccaacagagaatgctgaacgagttcccccatgccatcgccatgcagcacgccaaccagcccgatgagaccatcttccaggcagaagctcagtatttgcagatatatgctgtgactcccattccagagagccaggaggtcctgcagagagagggtgttccggacaacatcaaaagcttctataaagtgaatcacatctggaaattccgctatgaccgaccatttcacaaaggcacaaaagataaagagaatgaattcaagagtctctgggtggagagaacgtcattatacttggtgcagagtttgcctggcatctctcgctggtttgaagtggaaaagcgtgaagtggtagaaatgagtcctctggaaaatgcaattgaagtgctagaaaataagaatcagcagctgaagactctgattagtcagtgtcagacaagacagatgcagaatattaatcccctgactatgtgcctgaatggagttatagatgctgcagttaatggtggcgtttccaggtatcaagaggcattctttgtcaaagaatatatcttaagtcaccctgaagatggggagaaaattgcacgattaagagagctgatgcttgagcaggcacagattctggaatttggtttggccgtgcatgagaagtttgtacctcaagatatgagaccccttcacaaaaagctggttgaccaattctttgtgatgaagtcgagcttagggatacaggagttctctgcttgtatgcaagccagtcctgtccattttcctaatggaagccctcgtgtgtgtagaaactcagcacctgcttctgtgagcccagatggtaccagggtaattcctagacgcagcccgttaagttacccagctgtcaaccgatattcttcctcctcactgtcctcacaagcttctgctgaagtaagcaatattacagggcaatcagaaagctctgatgaagtctttaacatgcagccaagtccatctacctcaagcttgagttctactcactcggcttcacctaatgtgacaagttctgctccatcgagtgccagagcttctcctttgttgtctgacaaacacaaacattcccgagaaaactcttgcctgtcaccaagagagagaccatgcagtgccatctatccaacacctgtggagccttcgcagaggatgctgtttaatcatattggagacggggccttgccacgcagtgacccaaatctctctgcacctgaaaaagctgtgaaccccacccctagcagctggagcctggacagtgggaaggaagccaagaacatgtcggatagtgggaaacttatctctccccctgtccctccaagacccacacagactgcttcaccagcaagacacacgacatcagtatccccctcgcctgccgggcgatctccattgaagggctctgtgcagtctttcaccccctctccagtggagtaccactcgccaggactcatctccaactcccctgtcttgtcgggcagctacagcagtgggatttcttctctcagccggtgcagcacgtcggaaacctcaggctttgaaaatcaggtgaatgaacagtcggcccccctgccggtgccagtgccggtgcccgtgccgagctacggcggggaggagccagtgcgcaaggagagcaagactccgcccccgtacagcgtctacgagcggactctgcggcgccccgtcccgctacctcacagcctctccatccccgtcacgtcggagccgcccgcgctgccccccaagcctctggcagcgcgatccagccacctggagaatggggcccggaggactgaccccggcccgcggcccaggcccctgccccgcaaggtctctcagttataa

SEQ ID NO: 10 - protein, 1966 aa, Homo sapiens
MWIPTEHEKYGVVIASFRGTVPYGLSLEIGDTVQILEKCDGWYRGFALKNPNIKGIFPSSYVHLKNACVKNKGQFEMVIPTEDSVIIEMTSTLRDWGTMWKQLYVRNEGDLFHRLWHIMNEILDLRRQVLVGHLTHDRMKDVKRHITARLDWGNEQLGLDLVPRKEYAMVDPEDISITELYRLMEHRHRKKDTPVQASSHHLFVQMKSLMCSNLGEELEVIFSLFDSKENRPISERFFLRLNRNGLPKAPDKPERHCSLFVDLGSSELRKDIYITVHIIRIGRMGAGEKKNACSVQYRRPFGCAVLSIADLLTGETKDDLILKVYMCNTESEWYQIHENIIKKLNARYNLTGSNAGLAVSLQLLHGDIEQIRREYSSVFSHGVSITRKLGFSNIIMPGEMRNDLYITIERGEFEKGGKSVARNVEVTMFIVDSSGQTLKDFISFGSGEPPASEYHSFVLYHNNSPRWSELLKLPIPVDKFRGAHIRFEFRHCSTKEKGEKKLFGFSFVPLMQEDGRTLPDGTHELIVHKCEENTNLQDTTRYLKLPFSKGIFLGNNNQAMKATKESFCITSFLCSTKLTQNGDMLDLLKWRTHPDKITGCLSKLKEIDGSEIVKFLQDTLDTLFGILDENSQKYGSKVFDSLVHIINLLQDSKFHHFKPVMDTYIESHFAGALAYRDLIKVLKWYVDRITEAERQEHIQEVLKAQEYIFKYIVQSRRLFSLATGGQNEEEFRCCIQELLMSVRFFLSQESKGSGALSQSQAVFLSSFPAVYSELLKLFDVREVANLVQDTLGSLPTILHVDDSLQAIKLQCIGKTVESQLYTNPDSRYILLPVVLHHLHIHLQEQKDLIMCARILSNVFCLIKKNSSEKSVLEEIDVIVASLLDILLRTILEITSRPQPSSSAMRFQFQDVTGEFVACLLSLLRQMTDRHYQQLLDSFNTKEELRDFLLQIFTVFRILIRPEMFPKDWTVMRLVANNVIITTVLYLSDALRKNFLNENFDYKIWDSYFYLAVIFINQLCLQLEMFTPSKKKKVLEKYGDMRVTMGCEIFSMWQNLGEHKLHFIPALIGPFLEVTLIPQPGLRNVMIPIFHDMMDWEQRRSGNFKQVEAKLIDKLDSLMSEGKGDETYRELFNSILLKKIERETWRESGVSLIATVTRLMERLLDYRDCMKMGEVDGKKIGCTVSLLNFYKTELNKEEMYIRYIHKLYDLHLKAQNFTEAAYTLLLYDELLEWSDRPLREFLTYPMQTEWQRKEHLHLTIIQNFDRGKCWENGIILCRKIAEQYESYYDYRNLSKMRMMEASLYDKIMDQQRLEPEFFRVGFYGKKFPFFLRNKEFVCRGHDYERLEAFQQRMLNEFPHAIAMQHANQPDETIFQAEAQYLQIYAVTPIPESQEVLQREGVPDNIKSFYKVNHIWKFRYDRPFHKGTKDKENEFKSLWVERTSLYLVQSLPGISRWFEVEKREVVEMSPLENAIEVLENKNQQLKTLISQCQTRQMQNINPLTMCLNGVIDAAVNGGVSRYQEAFFVKEYILSHPEDGEKIARLRELMLEQAQILEFGLAVHEKFVPQDMRPLHKKLVDQFFVMKSSLGIQEFSACMQASPVHFPNGSPRVCRNSAPASVSPDGTRVIPRRSPLSYPAVNRYSSSSLSSQASAEVSNITGQSESSDEVFNMQPSPSTSSLSSTHSASPNVTSSAPSSARASPLLSDKHKHSRENSCLSPRERPCSAIYPTPVEPSQRMLFNHIGDGALPRSDPNLSAPEKAVNPTPSSWSLDSGKEAKNMSDSGKLISPPVPPRPTQTASPARHTTSVSPSPAGRSPLKGSVQSFTPSPVEYHSPGLISNSPVLSGSYSSGISSLSRCSTSETSGFENQVNEQSAPLPVPVPVPVPSYGGEEPVRKESKTPPPYSVYERTLRRPVPLPHSLSIPVTSEPPALPPKPLAARSSHLENGARRTDPGPRPRPLPRKVSQL

SEQ ID NO: 11 - DNA, 5901 bp, Homo sapiens
atgtggatacctacggagcacgagaaatacggcgtggttattgccagtttccgaggaaccgttccatatggcctgtcattggaaattggagatacagttcagatcctggagaagtgtgatggctggtacagaggatttgccttaaaaaacccaaatatcaagggtatatttccttccagctacgttcacttgaaaaatgcctgtgtaaagaacaaaggacaatttgaaatggttattcccactgaagactctgttatcacagaaatgacatcaacattaagagactggggaaccatgtggaaacaactctatgtgcgtaatgaaggcgatctcttccaccggctgtggcacatcatgaatgaaatcctggacctgaggcggcaggtgctggtgggccacctcacccacgaccggatgaaggacgtgaagcgccacattactgcccggcttgactggggcaatgaacaactgggactggacctggtgcctaggaaagagtacgcaatggtggatccggaagacatcagcattactgagctctaccgattgatggaacatcgacatcggaagaaagacaccccggtgcaggccagcagtcaccacctctttgtccagatgaagagcctcatgtgttccaacctgggagaggagctggaggtcatcttctcactctttgacagtaaagagaaccggccaatcagtgagagatttttcttgaggctgaatagaaacgggcttcccaaagcccctgataaaccggaacgacattgctccctctttgtggatttgggcagcagtgagctaagaaaggacatttatatcaccgtgcacattatccgaatcggtcgaatgggagcaggagaaaaaaagaatgcctgtagtgtccagtaccgacgaccctttggctgtgcagttcttagcatcgctgacctgctaacaggagagacaaaggatgacctcattctgaaagtatacatgtgtaacacagagagtgagtggtaccaaatccatgagaacatcatcaaaaagctgaatgcacgttataacttgactggctccaatgcaggtttagcagtttccttacagctattgcacggagacattgaacaaatcagaagggaatattcatcagtattttctcatggagtatccataacaaggaagctgggattttcaaatattattatgcctggtgaaatgaggaatgatttatatatcactattgaaaggggagaatttgagaaaggagggaagagcgtggccagaaatgtggaagttacgatgttcattgtagacagtagtggccaaaccctgaaggattttatctccttcggctctggggagccaccagccagtgagtaccactcctttgtgctttaccataacaacagtcccaggtggtctgaactgctgaaacttcccattcctgtggataaattccggggtgcacacatccgcttcgagtttcggcattgttccacaaaggagaaaggagagaagaagttgtttgggttttcttttgtccctctgatgcaagaagatggtaggactcttccagatggcactcatgagctcatcgtgcataagtgtgaagaaaacacaaatcttcaggatactacccgctacctcaaacttcccttttccaagggcattttccttgggaataataatcaagccatgaaggccacaaaggagtccttttgtattacatcttttctctgttccacaaaactcacacaaaatggtgatatgcttgatcttttgaaatggagaacccacccagacaagatcactggctgtctctctaaattaaaagaaattgatggctcagagatagtaaagtttctgcaggatacactggataccttatttggaattttagatgaaaattcccaaaaatatgggtctaaagtgtttgattctttggttcacataataaatttgctgcaagatagcaaatttcatcattttaaacctgtaatggacacttacattgagagtcattttgctggggcacttgcatacagagatctcatcaaagtgctcaaatggtacgtggaccggatcacagaagcagagcggcaagagcatatccaggaggtgctgaaggcacaagaatacatttttaagtatatagttcaatctcgaaggctgttttcccttgccactggtgggcaaaacgaagaggagttccgctgctgcattcaggagcttctcatgtcagtccgtttctttctttcgcaagagagcaaagggtctggagcattatctcagtcacaggctgtgtttctgagctctttccctgccgtgtactcagaactgttgaagctctttgatgtccgggaagtagccaacttggtccaggacaccctgggcagtctgccgaccatcctgcatgtggatgattccctgcaggccatcaaactgcagtgcattggcaaaaccgtggaaagccagctttataccaacccagattcccgatacattcttctgcctgtcgtgttacatcacctccacattcacttgcaagaacagaaggacctgatcatgtgtgcacgtatccttagcaacgtattttgtcttatcaagaaaaatagctcagaaaaatctgtgctggaggaaatagatgtgatagtggccagcttgctggatattctgctgaggaccatattggagatcaccagccgacctcagccatccagctcagcaatgcggttccagttccaggatgtcactggggagtttgttgcttgtctcctgtccctattacgacaaatgacagatagacattatcaacagcttcttgatagttttaatacaaaggaagaactaagggatttcctgctgcagatatttactgtgttccgaatattgatacgcccggagatgtttccaaaggactggactgttatgcgcttggttgctaacaatgttattattacaacagttctatacctctcagatgcacttcgtaagaacttcttaaatgaaaactttgattataagatctgggattcctacttttacctcgcagtcatttttataaaccagttgtgtctgcagttggagatgttcacaccttccaagaagaaaaaggtgttagaaaagtatggtgacatgcgggtaacaatgggttgtgaaattttcagcatgtggcaaaacctaggagagcacaagcttcattttatccctgccctgattggccccttcctagaagtgaccttgataccccagccaggtcttcggaatgtcatgattccaatttttcatgatatgatggactgggagcagaggcggagtggcaactttaaacaggtggaagcccagctaattgacaaactggatagcctgatgtcagaaggcaaaggtgacgaaacataccgcgagctcttcaacagtattctactaaagaaaattgagcgggaaacatggcgggaaagtggcgtttcattaattgctactgtaactcgtctaatggagaggttgttagattacagggactgcatgaaaatgggagaggtagatggcaaaaagattggctgcacagttagccttctgaacttctataagactgaactgaacaaggaggagatgtatatacgctacattcacaaactctatgatctgcatctcaaagcacagaactttacagaagctgcatataccctcctcttatatgacgagctactggaatggtctgatcggcccctcagggagttcctgacctaccccatgcaaacagaatggcagcgcaaagagcacctgcacctcaccatcatccagaactttgacagaggcaaatgttgggagaatggcattatcttgtgccggaagattgcagagcagtatgagagttattatgactacagaaacctgagcaagatgcggatgatggaagcctctttgtatgacaaaattatggaccagcaacgtcttgaaccagagttcttcagagttggattttatggaaaaaaatttccatttttcttaagaaataaggagtttgtgtgtcgagggcatgactacgagaggctggaagccttccaacagagaatgctgaacgagttcccccatgccatcgccatgcagcacgccaaccagcccgatgagaccatcttccaggcagaagctcagtatttgcagatatatgctgtgactcccattccagagagccaggaggtcctgcagagagagggtgttccggacaacatcaaaagcttctataaagtgaatcacatctggaaattccgctatgaccgaccatttcacaaaggcacaaaagataaagagaatgaattcaagagtctctgggtggagagaacgtcattatacttggtgcagagtttgcctggcatctctcgctggtttgaagtggaaaagcgtgaagtggtagaaatgagtcctctggaaaatgcaattgaagtgctagaaaataagaatcagcagctgaagactctgattagtcagtgtcagacaagacagatgcagaatattaatcccctgactatgtgcctgaatggagttatagatgctgcagttaatggtggcgtttccaggtatcaagaggcattctttgtcaaagaatatatcttaagtcaccctgaagatggggagaaaattgcacgattaagagagctgatgcttgagcaggcacagattctggaatttggtttggccgtgcatgagaagtttgtacctcaagatatgagaccccttcacaaaaagctggttgaccaattctttgtgatgaagtcgagcttagggatacaggagttctctgcttgtatgcaagccagtcctgtccattttcctaatggaagccctcgtgtgtgtagaaactcagcacctgcttctgtgagcccagatggtaccagggtaattcctagacgcagcccgttaagttacccagctgtcaaccgatattcttcctcctcactgtcctcacaagcttctgctgaagtaagcaatattacagggcaatcagaaagctctgatgaagtctttaacatgcagccaagtccatctacctcaagcttgagttctactcactcggcttcacctaatgtgacaagttctgctccatcgagtgccagagcttctcctttgttgtctgacaaacacaaacattcccgagaaaactcttgcctgtcaccaagagagagaccatgcagtgccatctatccaacacctgtggagccttcgcagaggatgctgtttaatcatattggagacggggccttgccacgcagtgacccaaatctctctgcacctgaaaaagctgtgaaccccacccctagcagctggagcctggacagtgggaaggaagccaagaacatgtcggatagtgggaaacttatctctccccctgtccctccaagacccacacagactgcttcaccagcaagacacacgacatcagtatccccctcgcctgccgggcgatctccattgaagggctctgtgcagtctttcaccccctctccagtggagtaccactcgccaggactcatctccaactcccctgtcttgtcgggcagctacagcagtgggatttcttctctcagccggtgcagcacgtcggaaacctcaggctttgaaaatcaggtgaatgaacagtcggcccccctgccggtgccagtgccggtgcccgtgccgagctacggcggggaggagccagtgcgcaaggagagcaagactccgcccccgtacagcgtctacgagcggactctgcggcgccccgtcccgctacctcacagcctctccatccccgtcacgtcggagccgcccgcgctgccccccaagcctctggcagcgcgatccagccacctggagaatggggcccggaggactgaccccggcccgcggcccaggcccctgccccgcaaggtctctcagttataa

SEQ ID NO: 12 - protein, 1966 aa, Homo sapiens
MWIPTEHEKYGVVIASFRGTVPYGLSLEIGDTVQILEKCDGWYRGFALKNPNIKGIFPSSYVHLKNACVKNKGQFEMVIPTEDSVITEMTSTLRDWGTMWKQLYVRNEGDLFHRLWHIMNEILDLRRQVLVGHLTHDRMKDVKRHITARLDWGNEQLGLDLVPRKEYAMVDPEDISITELYRLMEHRHRKKDTPVQASSHHLFVQMKSLMCSNLGEELEVIFSLFDSKENRPISERFFLRLNRNGLPKAPDKPERHCSLFVDLGSSELRKDIYITVHIIRIGRMGAGEKKNACSVQYRRPFGCAVLSIADLLTGETKDDLILKVYMCNTESEWYQIHENIIKKLNARYNLTGSNAGLAVSLQLLHGDIEQIRREYSSVFSHGVSITRKLGFSNIIMPGEMRNDLYITIERGEFEKGGKSVARNVEVTMFIVDSSGQTLKDFISFGSGEPPASEYHSFVLYHNNSPRWSELLKLPIPVDKFRGAHIRFEFRHCSTKEKGEKKLFGFSFVPLMQEDGRTLPDGTHELIVHKCEENTNLQDTTRYLKLPFSKGIFLGNNNQAMKATKESFCITSFLCSTKLTQNGDMLDLLKWRTHPDKITGCLSKLKEIDGSEIVKFLQDTLDTLFGILDENSQKYGSKVFDSLVHIINLLQDSKFHHFKPVMDTYIESHFAGALAYRDLIKVLKWYVDRITEAERQEHIQEVLKAQEYIFKYIVQSRRLFSLATGGQNEEEFRCCIQELLMSVRFFLSQESKGSGALSQSQAVFLSSFPAVYSELLKLFDVREVANLVQDTLGSLPTILHVDDSLQAIKLQCIGKTVESQLYTNPDSRYILLPVVLHHLHIHLQEQKDLIMCARILSNVFCLIKKNSSEKSVLEEIDVIVASLLDILLRTILEITSRPQPSSSAMRFQFQDVTGEFVACLLSLLRQMTDRHYQQLLDSFNTKEELRDFLLQIFTVFRILIRPEMFPKDWTVMRLVANNVIITTVLYLSDALRKNFLNENFDYKIWDSYFYLAVIFINQLCLQLEMFTPSKKKKVLEKYGDMRVTMGCEIFSMWQNLGEHKLHFIPALIGPFLEVTLIPQPGLRNVMIPIFHDMMDWEQRRSGNFKQVEATLIDKLDSLMSEGKGDETYRELFNSILLKKIERETWRESGVSLIATVTRLMERLLDYRDCMKMGEVDGKKIGCTVSLLNFYKTELNKEEMYIRYIHKLYDLHLKAQNFTEAAYTLLLYDELLEWSDRPLREFLTYPMQTEWQRKEHLHLTIIQNFDRGKCWENGIILCRKIAEQYESYYDYRNLSKMRMMEASLYDKIMDQQRLEPEFFRVGFYGKKFPFFLRNKEFVCRGHDYERLEAFQQRMLNEFPHAIAMQHANQPDETIFQAEAQYLQIYAVTPIPESQEVLQREGVPDNIKSFYKVNHIWKFRYDRPFHKGTKDKENEFKSLWVERTSLYLVQSLPGISRWFEVEKREVVEMSPLENAIEVLENKNQQLKTLISQCQTRQMQNINPLTMCLNGVIDAAVNGGVSRYQEAFFVKEYILSHPEDGEKIARLRELMLEQAQILEFGLAVHEKFVPQDMRPLHKKLVDQFFVMKSSLGIQEFSACMQASPVHFPNGSPRVCRNSAPASVSPDGTRVIPRRSPLSYPAVNRYSSSSLSSQASAEVSNITGQSESSDEVFNMQPSPSTSSLSSTHSASPNVTSSAPSSARASPLLSDKHKHSRENSCLSPRERPCSAIYPTPVEPSQRMLFNHIGDGALPRSDPNLSAPEKAVNPTPSSWSLDSGKEAKNMSDSGKLISPPVPPRPTQTASPARHTTSVSPSPAGRSPLKGSVQSFTPSPVEYHSPGLISNSPVLSGSYSSGISSLSRCSTSETSGFENQVNEQSAPLPVPVPVPVPSYGGEEPVRKESKTPPPYSVYERTLRRPVPLPHSLSIPVTSEPPALPPKPLAARSSHLENGARRTDPGPRPRPLPRKVSQL

SEQ ID NO: 13 - DNA, 5901 bp, Homo sapiens
atgtggatacctacggagcacgagaaatacggcgtggttattgccagtttccgaggaaccgttccatatggcctgtcattggaaattggagatacagttcagatcctggagaagtgtgatggctggtacagaggatttgccttaaaaaacccaaatatcaagggtatatttccttccagctacgttcacttgaaaaatgcctgtgtaaagaacaaaggacaatttgaaatggttattcccactgaagactctgttatcacagaaatgacatcaacattaagagactggggaaccatgtggaaacaactctatgtgcgtaatgaaggcgatctcttccaccggctgtggcacatcatgaatgaaatcctggacctgaggcggcaggtgctggtgggccacctcacccacgaccggatgaaggacgtgaagcgccacattactgcccggcttgactggggcaatgaacaactgggactggacctggtgcctaggaaagagtacgcaatggtggatccggaagacatcagcattactgagctctaccgattgatggaacatcgacatcggaagaaagacaccccggtgcaggccagcagtcaccacctctttgtccagatgaagagcctcatgtgttccaacctgggagaggagctggaggtcatcttctcactctttgacagtaaagagaaccggccaatcagtgagagatttttcttgaggctgaatagaaacgggcttcccaaagcccctgataaaccggaacgacattgctccctctttgtggatttgggcagcagtgagctaagaaaggacatttatatcaccgtgcacattatccgaatcggtcgaatgggagcaggagaaaaaaagaatgcctgtagtgtccagtaccgacgaccctttggctgtgcagttcttagcatcgctgacctgctaacaggagagacaaaggatgacctcattctgaaagtatacatgtgtaacacagagagtgagtggtaccaaatccatgagaacatcatcaaaaagctgaatgcacgttataacttgactggctccaatgcaggtttagcagtttccttacagctattgcacggagacattgaacaaatcagaagggaatattcatcagtattttctcatggagtatccataacaaggaagctgggattttcaaatattattatgcctggtgaaatgaggaatgatttatatatcactattgaaaggggagaatttgagaaaggagggaagagcgtggccagaaatgtggaagttacgatgttcattgtagacagtagtggccaaaccctgaaggattttatctccttcggctctggggagccaccagccagtgagtaccactcctttgtgctttaccataacaacagtcccaggtggtctgaactgctgaaacttcccattcctgtggataaattccggggtgcacacatccgcttcgagtttcggcattgttccacaaaggagaaaggagagaagaagttgtttgggttttcttttgtccctctgatgcaagaagatggtaggactcttccagatggcactcatgagctcatcgtgcataagtgtgaagaaaacacaaatcttcaggatactacccgctacctcaaacttcccttttccaagggcattttccttgggaataataatcaagccatgaaggccacaaaggagtccttttgtattacatcttttctctgttccacaaaactcacacaaaatggtgatatgcttgatcttttgaaatggagaacccacccagacaagatcactggctgtctctctaaattaaaagaaattgatggctcagagatagtaaagtttctgcaggatacactggataccttatttggaattttagatgaaaattcccaaaaatatgggtctaaagtgtttgattctttggttcacataataaatttgctgcaagatagcaaatttcatcattttaaacctgtaatggacacttacattgagagtcattttgctggggcacttgcatacagagatctcatcaaagtgctcaaatggtacgtggaccggatcacagaagcagagcggcaagagcatatccaggaggtgctgaaggcacaagaatacatttttaagtatatagttcaatctcgaaggctgttttcccttgccactggtgggcaaaacgaagaggagttccgctgctgcattcaggagcttctcatgtcagtccgtttctttctttcgcaagagagcaaagggtctggagcattatctcagtcacaggctgtgtttctgagctctttccctgccgtgtactcagaactgttgaagctctttgatgtccgggaagtagccaacttggtccaggacaccctgggcagtctgccgaccatcctgcatgtggatgattccctgcaggccatcaaactgcagtgcattggcaaaaccgtggaaagccagctttataccaacccagattcccgatacattcttctgcctgtcgtgttacatcacctccacattcacttgcaagaacagaaggacctgatcatgtgtgcacgtatccttagcaacgtattttgtcttatcaagaaaaatagctcagaaaaatctgtgctggaggaaatagatgtgatagtggccagcttgctggatattctgctgaggaccatattggagatcaccagccgacctcagccatccagctcagcaatgcggttccagttccaggatgtcactggggagtttgttgcttgtctcctgtccctattacgacaaatgacagatagacattatcaacagcttcttgatagttttaatacaaaggaagaactaagggatttcctgctgcagatatttactgtgttccgaatattgatacgcccggagatgtttccaaaggactggactgttatgcgcttggttgctaacaatgttattattacaacagttctatacctctcagatgcacttcgtaagaacttcttaaatgaaaactttgattataagatctgggattcctacttttacctcgcagtcatttttataaaccagttgtgtctgcagttggagatgttcacaccttccaagaagaaaaaggtgttagaaaagtatggtgacatgcgggtaacaatgggttgtgaaattttcagcatgtggcaaaacctaggagagcacaagcttcattttatccctgccctgattggccccttcctagaagtgaccttgataccccagccaggtcttcggaatgtcatgattccaatttttcatgatatgatggactgggagcagaggcggagtggcaactttaaacaggtggaagccaagctaattgacaaactggatagcctgatgtcagaaggcaaaggtgacgaaacataccgcgagctcttcaacagtattctactaaagaaaattgagcgggaaacatggcgggaaagtggcgtttcattaattgctactgtaactcgtctaatggagaggttgttagattacagggactgcatgaaaatgggagaggtagatggcaaaaagattggctgcacagttagccttctgaacttctataagactgaactgaacaaggaggagatgtatatacgctacattcacaaactctatgatctgcatctcaaagcacagaactttacagaagctgcatataccctcctcttatatgacgagctactggaatggtctgatcggcccctcagggagttcctgacctaccccatgcaaacagaatggcagcgcaaagagcacctgcacctcaccatcatccagaactttgacagaggcaaatgttgggagaatggcattatcttgtgccggaagattgcagagcagtatgagagttattatgactacagaaacctgagcaagatgcggatgatggaagcctctttgtatgacaaaattatggaccagcaacgtcttgaaccagagttcttcagagttggattttatggaaaaaaatttccatttttcttaagaaataaggagtttgtgtgtcgagggcatgactacgagaggctggaagccttccaacagagaatgctgaacgagttcccccatgccatcgccatgcagcacgccaaccagcccgatgagaccatcttccaggcagaagctcagtatttgcagatatatgctgtgactcccattccagagagccaggaggtcctgcagagagagggtgttccggacaacatcaaaagcttctataaagtgaatcacatctggaaattccgctatgaccgaccatttcacaaaggcacaaaagataaagagaatgaattcaagagtctctgggtggagagaacgtcattatacttggtgcagagtttgcctggcatctctcgctggtttgaagtggaaaagcgtgaagtggtagaaatgagtcctctggaaaatgcaattgaagtgctagaaaataagaatcagcagctgaagactctgattagtcagtgtcagacaagacagatgcagaatattaatcccctgactatgtgcctgaatggagttatagatgctgcagttaatggtggcgtttccaggtatcaagaggcattctttgtcaaagaatatatcttaagtcaccctgaagatggggagaaaattgcacgattaagagagctgatgcttgagcaggcacagattctggaatttggtttggccgtgcatgagaagtttgtacctcaagatatgagaccccttcacaaaaagctggttgaccaattctttgtgatgaagtcgagcttagggatacaggagttctctgcttgtatgcaagccagtcctgtccattttcctaatggaagccctcgtgtgtgtagaaactcagcacctgcttctgtgagcccagatggtaccagggtaattcctagacgcagcccgttaagttacccagctgtcaaccgatattcttcctcctcactgtcctcacaagcttctgctgaagtaagcaatattacagggcaatcagaaagctctgatgaagtctttaacatgcagccaagtccatctacctcaagcttgagttctactcactcggcttcacctaatgtgacaagttctgctccatcgagtgccagagcttctcctttgttgtctgacaaacacaaacattcccgagaaaactcttgcctgtcaccaagagagagaccatgcagtgccatctatccaacacctgtggagccttcgcagaggatgctgtttaatcatattggagacggggccttgccacgcagtgacccaaatctccctgcacctgaaaaagctgtgaaccccacccctagcagctggagcctggacagtgggaaggaagccaagaacatgtcggatagtgggaaacttatctctccccctgtccctccaagacccacacagactgcttcaccagcaagacacacgacatcagtatccccctcgcctgccgggcgatctccattgaagggctctgtgcagtctttcaccccctctccagtggagtaccactcgccaggactcatctccaactcccctgtcttgtcgggcagctacagcagtgggatttcttctctcagccggtgcagcacgtcggaaacctcaggctttgaaaatcaggtgaatgaacagtcggcccccctgccggtgccagtgccggtgcccgtgccgagctacggcggggaggagccagtgcgcaaggagagcaagactccgcccccgtacagcgtctacgagcggactctgcggcgccccgtcccgctacctcacagcctctccatccccgtcacgtcggagccgcccgcgctgccccccaagcctctggcagcgcgatccagccacctggagaatggggcccggaggactgaccccggcccgcggcccaggcccctgccccgcaaggtctctcagttataa

SEQ ID NO: 14 - protein, 1966 aa, Homo sapiens
MWIPTEHEKYGVVIASFRGTVPYGLSLEIGDTVQILEKCDGWYRGFALKNPNIKGIFPSSYVHLKNACVKNKGQFEMVIPTEDSVITEMTSTLRDWGTMWKQLYVRNEGDLFHRLWHIMNEILDLRRQVLVGHLTHDRMKDVKRHITARLDWGNEQLGLDLVPRKEYAMVDPEDISITELYRLMEHRHRKKDTPVQASSHHLFVQMKSLMCSNLGEELEVIFSLFDSKENRPISERFFLRLNRNGLPKAPDKPERHCSLFVDLGSSELRKDIYITVHIIRIGRMGAGEKKNACSVQYRRPFGCAVLSIADLLTGETKDDLILKVYMCNTESEWYQIHENIIKKLNARYNLTGSNAGLAVSLQLLHGDIEQIRREYSSVFSHGVSITRKLGFSNIIMPGEMRNDLYITIERGEFEKGGKSVARNVEVTMFIVDSSGQTLKDFISFGSGEPPASEYHSFVLYHNNSPRWSELLKLPIPVDKFRGAHIRFEFRHCSTKEKGEKKLFGFSFVPLMQEDGRTLPDGTHELIVHKCEENTNLQDTTRYLKLPFSKGIFLGNNNQAMKATKESFCITSFLCSTKLTQNGDMLDLLKWRTHPDKITGCLSKLKEIDGSEIVKFLQDTLDTLFGILDENSQKYGSKVFDSLVHIINLLQDSKFHHFKPVMDTYIESHFAGALAYRDLIKVLKWYVDRITEAERQEHIQEVLKAQEYIFKYIVQSRRLFSLATGGQNEEEFRCCIQELLMSVRFFLSQESKGSGALSQSQAVFLSSFPAVYSELLKLFDVREVANLVQDTLGSLPTILHVDDSLQAIKLQCIGKTVESQLYTNPDSRYILLPVVLHHLHIHLQEQKDLIMCARILSNVFCLIKKNSSEKSVLEEIDVIVASLLDILLRTILEITSRPQPSSSAMRFQFQDVTGEFVACLLSLLRQMTDRHYQQLLDSFNTKEELRDFLLQIFTVFRILIRPEMFPKDWTVMRLVANNVIITTVLYLSDALRKNFLNENFDYKIWDSYFYLAVIFINQLCLQLEMFTPSKKKKVLEKYGDMRVTMGCEIFSMWQNLGEHKLHFIPALIGPFLEVTLIPQPGLRNVMIPIFHDMMDWEQRRSGNFKQVEAKLIDKLDSLMSEGKGDETYRELFNSILLKKIERETWRESGVSLIATVTRLMERLLDYRDCMKMGEVDGKKIGCTVSLLNFYKTELNKEEMYIRYIHKLYDLHLKAQNFTEAAYTLLLYDELLEWSDRPLREFLTYPMQTEWQRKEHLHLTIIQNFDRGKCWENGIILCRKIAEQYESYYDYRNLSKMRMMEASLYDKIMDQQRLEPEFFRVGFYGKKFPFFLRNKEFVCRGHDYERLEAFQQRMLNEFPHAIAMQHANQPDETIFQAEAQYLQIYAVTPIPESQEVLQREGVPDNIKSFYKVNHIWKFRYDRPFHKGTKDKENEFKSLWVERTSLYLVQSLPGISRWFEVEKREVVEMSPLENAIEVLENKNQQLKTLISQCQTRQMQNINPLTMCLNGVIDAAVNGGVSRYQEAFFVKEYILSHPEDGEKIARLRELMLEQAQILEFGLAVHEKFVPQDMRPLHKKLVDQFFVMKSSLGIQEFSACMQASPVHFPNGSPRVCRNSAPASVSPDGTRVIPRRSPLSYPAVNRYSSSSLSSQASAEVSNITGQSESSDEVFNMQPSPSTSSLSSTHSASPNVTSSAPSSARASPLLSDKHKHSRENSCLSPRERPCSAIYPTPVEPSQRMLFNHIGDGALPRSDPNLPAPEKAVNPTPSSWSLDSGKEAKNMSDSGKLISPPVPPRPTQTASPARHTTSVSPSPAGRSPLKGSVQSFTPSPVEYHSPGLISNSPVLSGSYSSGISSLSRCSTSETSGFENQVNEQSAPLPVPVPVPVPSYGGEEPVRKESKTPPPYSVYERTLRRPVPLPHSLSIPVTSEPPALPPKPLAARSSHLENGARRTDPGPRPRPLPRKVSQL

SEQ ID NO: 15 - DNA, 5901 bp, Homo sapiens
atgtggatacctacggagcacgagaaatacggcgtggttattgccagtttccgaggaaccgttccatatggcctgtcattggaaattggagatacagttcagatcctggagaagtgtgatggctggtacagaggatttgccttaaaaaacccaaatatcaagggtatatttccttccagctacgttcacttgaaaaatgcctgtgtaaagaacaaaggacaatttgaaatggttattcccactgaagactctgttatcacagaaatgacatcaacattaagagactggggaaccatgtggaaacaactctatgtgcgtaatgaaggcgatctcttccaccggctgtggcacatcatgaatgaaatcctggacctgaggcggcaggtgctggtgggccacctcacccacgaccggatgaaggacgtgaagcgccacattactgcccggcttgactggggcaatgaacaactgggactggacctggtgcctaggaaagagtacgcaatggtggatccggaagacatcagcattactgagctctaccgattgatggaacatcgacatcggaagaaagacaccccggtgcaggccagcagtcaccacctctttgtccagatgaagagcctcatgtgttccaacctgggagaggagctggaggtcatcttctcactctttgacagtaaagagaaccggccaatcagtgagagatttttcttgaggctgaatagaaacgggcttcccaaagcccctgataaaccggaacgacattgctccctctttgtggatttgggcagcagtgagctaagaaaggacatttatatcaccgtgcacattatccgaatcggtcgaatgggagcaggagaaaaaaagaatgcctgtagtgtccagtaccgacgaccctttggctgtgcagttcttagcatcgctgacctgctaacaggagagacaaaggatgacctcattctgaaagtatacatgtgtaacacagagagtgagtggtaccaaatccatgagaacatcatcaaaaagctgaatgcacgttataacttgactggctccaatgcaggtttagcagtttccttacagctattgcacggagacattgaacaaatcagaagggaatattcatcagtattttctcatggagtatccataacaaggaagctgggattttcaaatattattatgcctggtgaaatgaggaatgatttatatatcactattgaaaggggagaatttgagaaaggagggaagagcgtggccagaaatgtggaagttacgatgttcattgtagacagtagtggccaaaccctgaaggattttatctccttcggctctggggagccaccagccagtgagtaccactcctttgtgctttaccataacaacagtcccaggtggtctgaactgctgaaacttcccattcctgtggataaattccggggtgcacacatccgcttcgagtttcggcattgttccacaaaggagaaaggagagaagaagttgtttgggttttcttttgtccctctgatgcaagaagatggtaggactcttccagatggcactcatgagctcatcgtgcataagtgtgaagaaaacacaaatcttcaggatactacccgctacctcaaacttcccttttccaagggcattttccttgggaataataatcaagccatgaaggccacaaaggagtccttttgtattacatcttttctctgttccacaaaactcacacaaaatggtgatatgcttgatcttttgaaatggagaacccacccagacaagatcactggctgtctctctaaattaaaagaaattgatggctcagagatagtaaagtttctgcaggatacactggataccttatttggaattttagatgaaaattcccaaaaatatgggtctaaagtgtttgattctttggttcacataataaatttgctgcaagatagcaaatttcatcattttaaacctgtaatggacacttacattgagagtcattttgctggggcacttgcatacagagatctcatcaaagtgctcaaatggtacgtggaccggatcacagaagcagagcggcaagagcatatccaggaggtgctgaaggcacaagaatacatttttaagtatatagttcaatctcgaaggctgttttcccttgccactggtgggcaaaacgaagaggagttccgctgctgcattcaggagcttctcatgtcagtccgtttctttctttcgcaagagagcaaagggtctggagcattatctcagtcacaggctgtgtttctgagctctttccctgccgtgtactcagaactgttgaagctctttgatgtccgggaagtagccaacttggtccaggacaccctgggcagtctgccgaccatcctgcatgtggatgattccctgcaggccatcaaactgcagtgcattggcaaaaccgtggaaagccagctttataccaacccagattcccgatacattcttctgcctgtcgtgttacatcacctccacattcacttgcaagaacagaaggacctgatcatgtgtgcacgtatccttagcaacgtattttgtcttatcaagaaaaatagctcagaaaaatctgtgctggaggaaatagatgtgatagtggccagcttgctggatattctgctgaggaccatattggagatcaccagccgacctcagccatccagctcagcaatgcggttccagttccaggatgtcactggggagtttgttgcttgtctcctgtccctattacgacaaatgacagatagacattatcaacagcttcttgatagttttaatacaaaggaagaactaagggatttcctgctgcagatatttactgtgttccgaatattgatacgcccggagatgtttccaaaggactggactgttatgcgcttggttgctaacaatgttattattacaacagttctatacctctcagatgcacttcgtaagaacttcttaaatgaaaactttgattataagatctgggattcctacttttacctcgcagtcatttttataaaccagttgtgtctgcagttggagatgttcacaccttccaagaagaaaaaggtgttagaaaagtatggtgacatgcgggtaacaatgggttgtgaaattttcagcatgtggcaaaacctaggagagcacaagcttcattttatccctgccctgattggccccttcctagaagtgaccttgataccccagccaggtcttcggaatgtcatgattccaatttttcatgatatgatggactgggagcagaggcggagtggcaactttaaacaggtggaagccaagctaattgacaaactggatagcctgatgtcagaaggcaaaggtgacgaaacataccgcgagctcttcaacagtattctactaaagaaaattgagcgggaaacatggcgggaaagtggcgtttcattaattgctactgtaactcgtctaatggagaggttgttagattacagggactgcatgaaaatgggagaggtagatggcaaaaagattggctgcacagttagccttctgaacttctataagactgaactgaacaaggaggagatgtatatacgctacattcacaaactctatgatctgcatctcaaagcacagaactttacagaagctgcatataccctcctcttatatgacgagctactggaatggtctgatcggcccctcagggagttcctgacctaccccatgcaaacagaatggcagcgcaaagagcacctgcacctcaccatcatccagaactttgacagaggcaaatgttgggagaatggcattatcttgtgccggaagattgcagagcagtatgagagttattatgactacagaaacctgagcaagatgcggatgatggaagcctctttgtatgacaaaattatggaccagcaacgtcttgaaccagagttcttcagagttggattttatggaaaaaaatttccatttttcttaagaaataaggagtttgtgtgtcgagggcatgactacgagaggctggaagccttccaacagagaatgctgaacgagttcccccatgccatcgccatgcagcacgccaaccagcccgatgagaccatcttccaggcagaagctcagtatttgcagatatatgctgtgactcccattccagagagccaggaggtcctgcagagagagggtgttccggacaacatcaaaagcttctataaagtgaatcacatctggaaattccgctatgaccgaccatttcacaaaggcacaaaagataaagagaatgaattcaagagtctctgggtggagagaacgtcattatacttggtgcagagtttgcctggcatctctcgctggtttgaagtggaaaagcgtgaagtggtagaaatgagtcctctggaaaatgcaattgaagtgctagaaaataagaatcagcagctgaagactctgattagtcagtgtcagacaagacagatgcagaatattaatcccctgactatgtgcctgaatggagttatagatgctgcagttaatggtggcgtttccaggtatcaagaggcattctttgtcaaagaatatatcttaagtcaccctgaagatggggagaaaattgcacgattaagagagctgatgcttgagcaggcacagattctggaatttggtttggccgtgcatgagaagtttgtacctcaagatatgagaccccttcacaaaaagctggttgaccaattctttgtgatgaagtcgagcttagggatacaggagttctctgcttgtatgcaagccagtcctgtccattttcctaatggaagccctcgtgtgtgtagaaactcagcacctgcttctgtgagcccagatggtaccagggtaattcctagacgcagcccgttaagttacccagctgtcaaccgatattcttcctcctcactgtcctcacaagcttctgctgaagtaagcaatattacagggcaatcagaaagctctgatgaagtctttaacatgcagccaagtccatctacctcaagcttgagttctactcactcggcttcacctaatgtgacaagttctgctccatcgagtgccagagcttctcctttgttgtctgacaaacacaaacattcccgagaaaactcttgcctgtcactaagagagagaccatgcagtgccatctatccaacacctgtggagccttcgcagaggatgctgtttaatcatattggagacggggccttgccacgcagtgacccaaatctctctgcacctgaaaaagctgtgaaccccacccctagcagctggagcctggacagtgggaaggaagccaagaacatgtcggatagtgggaaacttatctctccccctgtccctccaagacccacacagactgcttcaccagcaagacacacgacatcagtatccccctcgcctgccgggcgatctccattgaagggctctgtgcagtctttcaccccctctccagtggagtaccactcgccaggactcatctccaactcccctgtcttgtcgggcagctacagcagtgggatttcttctctcagccggtgcagcacgtcggaaacctcaggctttgaaaatcaggtgaatgaacagtcggcccccctgccggtgccagtgccggtgcccgtgccgagctacggcggggaggagccagtgcgcaaggagagcaagactccgcccccgtacagcgtctacgagcggactctgcggcgccccgtcccgctacctcacagcctctccatccccgtcacgtcggagccgcccgcgctgccccccaagcctctggcagcgcgatccagccacctggagaatggggcccggaggactgaccccggcccgcggcccaggcccctgccccgcaaggtctctcagttataa

SEQ ID NO: 16 - protein, 1966 aa, Homo sapiens
MWIPTEHEKYGVVIASFRGTVPYGLSLEIGDTVQILEKCDGWYRGFALKNPNIKGIFPSSYVHLKNACVKNKGQFEMVIPTEDSVITEMTSTLRDWGTMWKQLYVRNEGDLFHRLWHIMNEILDLRRQVLVGHLTHDRMKDVKRHITARLDWGNEQLGLDLVPRKEYAMVDPEDISITELYRLMEHRHRKKDTPVQASSHHLFVQMKSLMCSNLGEELEVIFSLFDSKENRPISERFFLRLNRNGLPKAPDKPERHCSLFVDLGSSELRKDIYITVHIIRIGRMGAGEKKNACSVQYRRPFGCAVLSIADLLTGETKDDLILKVYMCNTESEWYQIHENIIKKLNARYNLTGSNAGLAVSLQLLHGDIEQIRREYSSVFSHGVSITRKLGFSNIIMPGEMRNDLYITIERGEFEKGGKSVARNVEVTMFIVDSSGQTLKDFISFGSGEPPASEYHSFVLYHNNSPRWSELLKLPIPVDKFRGAHIRFEFRHCSTKEKGEKKLFGFSFVPLMQEDGRTLPDGTHELIVHKCEENTNLQDTTRYLKLPFSKGIFLGNNNQAMKATKESFCITSFLCSTKLTQNGDMLDLLKWRTHPDKITGCLSKLKEIDGSEIVKFLQDTLDTLFGILDENSQKYGSKVFDSLVHIINLLQDSKFHHFKPVMDTYIESHFAGALAYRDLIKVLKWYVDRITEAERQEHIQEVLKAQEYIFKYIVQSRRLFSLATGGQNEEEFRCCIQELLMSVRFFLSQESKGSGALSQSQAVFLSSFPAVYSELLKLFDVREVANLVQDTLGSLPTILHVDDSLQAIKLQCIGKTVESQLYTNPDSRYILLPVVLHHLHIHLQEQKDLIMCARILSNVFCLIKKNSSEKSVLEEIDVIVASLLDILLRTILEITSRPQPSSSAMRFQFQDVTGEFVACLLSLLRQMTDRHYQQLLDSFNTKEELRDFLLQIFTVFRILIRPEMFPKDWTVMRLVANNVIITTVLYLSDALRKNFLNENFDYKIWDSYFYLAVIFINQLCLQLEMFTPSKKKKVLEKYGDMRVTMGCEIFSMWQNLGEHKLHFIPALIGPFLEVTLIPQPGLRNVMIPIFHDMMDWEQRRSGNFKQVEAKLIDKLDSLMSEGKGDETYRELFNSILLKKIERETWRESGVSLIATVTRLMERLLDYRDCMKMGEVDGKKIGCTVSLLNFYKTELNKEEMYIRYIHKLYDLHLKAQNFTEAAYTLLLYDELLEWSDRPLREFLTYPMQTEWQRKEHLHLTIIQNFDRGKCWENGIILCRKIAEQYESYYDYRNLSKMRMMEASLYDKIMDQQRLEPEFFRVGFYGKKFPFFLRNKEFVCRGHDYERLEAFQQRMLNEFPHAIAMQHANQPDETIFQAEAQYLQIYAVTPIPESQEVLQREGVPDNIKSFYKVNHIWKFRYDRPFHKGTKDKENEFKSLWVERTSLYLVQSLPGISRWFEVEKREVVEMSPLENAIEVLENKNQQLKTLISQCQTRQMQNINPLTMCLNGVIDAAVNGGVSRYQEAFFVKEYILSHPEDGEKIARLRELMLEQAQILEFGLAVHEKFVPQDMRPLHKKLVDQFFVMKSSLGIQEFSACMQASPVHFPNGSPRVCRNSAPASVSPDGTRVIPRRSPLSYPAVNRYSSSSLSSQASAEVSNITGQSESSDEVFNMQPSPSTSSLSSTHSASPNVTSSAPSSARASPLLSDKHKHSRENSCLSLRERPCSAIYPTPVEPSQRMLFNHIGDGALPRSDPNLSAPEKAVNPTPSSWSLDSGKEAKNMSDSGKLISPPVPPRPTQTASPARHTTSVSPSPAGRSPLKGSVQSFTPSPVEYHSPGLISNSPVLSGSYSSGISSLSRCSTSETSGFENQVNEQSAPLPVPVPVPVPSYGGEEPVRKESKTPPPYSVYERTLRRPVPLPHSLSIPVTSEPPALPPKPLAARSSHLENGARRTDPGPRPRPLPRKVSQL

SEQ ID NO: 17 - DNA, 5901 bp, Homo sapiens; identical to SEQ ID NO: 15

SEQ ID NO: 18 - protein, 1966 aa, Homo sapiens; identical to SEQ ID NO: 16

SEQ ID NO: 19 - DNA, 5901 bp, Homo sapiens
atgtggatacctacggagcacgagaaatacggcgtggttattgccagtttccgaggaaccgttccatatggcctgtcattggaaattggagatacagttcagatcctggagaagtgtgatggctggtacagaggatttgccttaaaaaacccaaatatcaagggtatatttccttccagctacgttcacttgaaaaatgcctgtgtaaagaacaaaggacaatttgaaatggttattcccactgaagactctgttatcacagaaatgacatcaacattaagagactggggaaccatgtggaaacaactctatgtgcgtaatgaaggcgatctcttccaccggctgtggcacatcatgaatgaaatcctggacctgaggcggcaggtgctggtgggccacctcacccacgaccggatgaaggacgtgaagcgccacattactgcccggcttgactggggcaatgaacaactgggactggacctggtgcctaggaaagagtacgcaatggtggatccggaagacatcagcattactgagctctaccgattgatggaacatcgacatcggaagaaagacaccccggtgcaggccagcagtcaccacctctttgtccagatgaagagcctcatgtgttccaacctgggagaggagctggaggtcatcttctcactctttgacagtaaagagaaccggccaatcagtgagagatttttcttgaggctgaatagaaacgggcttcccaaagcccctgataaaccggaacgacattgctccctctttgtggatttgggcagcagtgagctaagaaaggacatttatatcaccgtgcacattatccgaatcggtcgaatgggagcaggagaaaaaaagaatgcctgtagtgtccagtaccgacgaccctttggctgtgcagttcttagcatcgctgacctgctaacaggagagacaaaggatgacctcattctgaaagtatacatgtgtaacacagagagtgagtggtaccaaatccatgagaacatcatcaaaaagctgaatgcacgttataacttgactggctccaatgcaggtttagcagtttccttacagctattgcacggagacattgaacaaatcagaagggaatattcatcagtattttctcatggagtatccataacaaggaagctgggattttcaaatattattatgcctggtgaaatgaggaatgatttatatatcactattgaaaggggagaatttgagaaaggagggaagagcgtggccagaaatgtggaagttacgatgttcattgtagacagtagtggccaaaccctgaaggattttatctccttcggctctggggagccaccagccagtgagtaccactcctttgtgctttaccataacaacagtcccaggtggtctgaactgctgaaacttcccattcctgtggataaattccggggtgcacacatccgcttcgagtttcggcattgttccacaaaggagaaaggagagaagaagttgtttgggttttcttttgtccctctgatgcaagaagatggtaggactcttccagatggcactcatgagctcatcgtgcataagtgtgaagaaaacacaaatcttcaggatactacccgctacctcaaacttcccttttccaagggcattttccttgggaataataatcaagccatgaaggccacaaaggagtccttttgtattacatcttttctctgttccacaaaactcacacaaaatggtgatatgcttgatcttttgaaatggagaacccacccagacaagatcactggctgtctctctaaattaaaagaaattgatggctcagagatagtaaagtttctgcaggatacactggataccttatttggaattttagatgaaaattcccaaaaatatgggtctaaagtgtttgattctttggttcacataataaatttgctgcaagatagcaaatttcatcattttaaacctgtaatggacacttacattgagagtcattttgctggggcacttgcatacagagatctcatcaaagtgctcaaatggtacgtggaccggatcacagaagcagagcggcaagagcatatccaggaggtgctgaaggcacaagaatacatttttaagtatatagttcaatctcgaaggctgttttcccttgccactggtgggcaaaacgaagaggagttccgctgctgcattcaggagcttctcatgtcagtccgtttctttctttcgcaagagagcaaagggtctggagcattatctcagtcacaggctgtgtttctgagctctttccctgccgtgtactcagaactgttgaagctctttgatgtccgggaagtagccaacttggtccaggacaccctgggcagtctgccgaccatcctgcatgtggatgattccctgcaggccatcaaactgcagtgcattggcaaaaccgtggaaagccagctttataccaacccagattcccgatacattcttctgcctgtcgtgttacatcacctccacattcacttgcaagaacagaaggacctgatcatgtgtgcacgtatccttagcaacgtattttgtcttatcaagaaaaatagctcagaaaaatctgtgctggaggaaatagatgtgatagtggccagcttgctggatattctgctgaggaccatattggagatcaccagccgacctcagccatccagctcagcaatgcggttccagttccaggatgtcactggggagtttgttgcttgtctcctgtccctattacgacaaatgacagatagacattatcaacagcttcttgatagttttaatacaaaggaagaactaagggatttcctgctgcagatatttactgtgttccgaatattgatacgcccggagatgtttccaaaggactggactgttatgcgcttggttgctaacaatgttattattacaacagttctatacctctcagatgcacttcgtaagaacttcttaaatgaaaactttgattataagatctgggattcctacttttacctcgcagtcatttttataaaccagttgtgtctgcagttggagatgttcacaccttccaagaagaaaaaggtgttagaaaagtatggtgacatgcgggtaacaatgggttgtgaaattttcagcatgtggcaaaacctaggagagcacaagcttcattttatccctgccctgattggccccttcctagaagtgaccttgataccccagccaggtcttcggaatgtcatgattccaatttttcatgatatgatggactgggagcagaggcggagtggcaactttaaacaggtggaagccaagctaattgacaaactggatagcctgatgtcagaaggcaaaggtgacgaaacataccgcgagctcttcaacagtattctactaaagaaaattgagcgggaaacatggcgggaaagtggcgtttcattaattgctactgtaactcgtctaatggagaggttgttagattacagggactgcatgaaaatgggagaggtagatggcaaaaagattggctgcacagttagccttctgaacttctataagactgaactgaacaaggaggagatgtatatacgctacattcacaaactctatgatctgcatctcaaagcacagaactttacagaagctgcatataccctcctcttatatgacgagctactggaatggtctgatcggcccctcagggagttcctgacctaccccatgcaaacagaatggcagcgcaaagagcacctgcacctcaccatcatccagaactttgacagaggcaaatgttgggagaatggcattatcttgtgccggaagattgcagagcagtatgagagttattatgactacagaaacctgagcaagatgcggatgatggaagcctctttgtatgacaaaattatggaccagcaacgtcttgaaccagagttcttcagagttggattttatggaaaaaaatttccatttttcttaagaaataaggagtttgtgtgtcgagggcatgactacgagaggctggaagccttccaacagagaatgctgaacgagttcccccatgccatcgccatgcagcacgccaaccagcccgatgagaccatcttccaggcagaagctcagtatttgcagatatatgctgtgactcccattccagagagccaggaggtcctgcagagagagggtgttccggacaacatcaaaagcttctataaagtgaatcacatctggaaattccgctatgaccgaccatttcacaaaggcacaaaagataaagagaatgaattcaagagtctctgggtggagagaacgtcattatacttggtgcagagtttgcctggcatctctcgctggtttgaagtggaaaagcgtgaagtggtagaaatgagtcctctggaaaatgcaattgaagtgctagaaaataagaatcagcagctgaagactctgattagtcagtgtcagacaagacagatgcagaatattaatcccctgactatgtgcctgaatggagttatagatgctgcagttaatggtggcgtttccaggtatcaagaggcattctttgtcaaagaatatatcttaagtcaccctgaagatggggagaaaattgcacgattaagagagctgatgcttgagcaggcacagattctggaatttggtttggccgtgcatgagaagtttgtacctcaagatatgagaccccttcacaaaaagctggttgaccaattctttgtgatgaagtcgagcttagggatacaggagttctctgcttgtatgcaagccagtcctgtccattttcctaatggaagccctcgtgtgtgtagaaactcagcacctgcttctgtgagcccagatggtaccagggtaattcctagacgcagcccgttaagttacccagctgtcaaccgatattcttcctcctcactgtcctcacaagcttctgctgaagtaagcaatattacagggcaatcagaaagctctgatgaagtctttaacatgcagccaagtccatctacctcaagcttgagttctactcactcggcttcacctaatgtgacaagttctgctccatcgagtgccagagcttctcctttgttgtctgacaaacacaaacattcccgagaaaactcttgcctgtcaccaagagagagaccatgcagtgccatctatccaacacctgtggagcgttcgcagaggatgctgtttaatcatattggagacggggccttgccacgcagtgacccaaatctctctgcacctgaaaaagctgtgaaccccacccctagcagctggagcctggacagtgggaaggaagccaagaacatgtcggatagtgggaaacttatctctccccctgtccctccaagacccacacagactgcttcaccagcaagacacacgacatcagtatccccctcgcctgccgggcgatctccattgaagggctctgtgcagtctttcaccccctctccagtggagtaccactcgccaggactcatctccaactcccctgtcttgtcgggcagctacagcagtgggatttcttctctcagccggtgcagcacgtcggaaacctcaggctttgaaaatcaggtgaatgaacagtcggcccccctgccggtgccagtgccggtgcccgtgccgagctacggcggggaggagccagtgcgcaaggagagcaagactccgcccccgtacagcgtctacgagcggactctgcggcgccccgtcccgctacctcacagcctctccatccccgtcacgtcggagccgcccgcgctgccccccaagcctctggcagcgcgatccagccacctggagaatggggcccggaggactgaccccggcccgcggcccaggcccctgccccgcaaggtctctcagttataa

SEQ ID NO: 20 - protein, 1966 aa, Homo sapiens
MWIPTEHEKYGVVIASFRGTVPYGLSLEIGDTVQILEKCDGWYRGFALKNPNIKGIFPSSYVHLKNACVKNKGQFEMVIPTEDSVITEMTSTLRDWGTMWKQLYVRNEGDLFHRLWHIMNEILDLRRQVLVGHLTHDRMKDVKRHITARLDWGNEQLGLDLVPRKEYAMVDPEDISITELYRLMEHRHRKKDTPVQASSHHLFVQMKSLMCSNLGEELEVIFSLFDSKENRPISERFFLRLNRNGLPKAPDKPERHCSLFVDLGSSELRKDIYITVHIIRIGRMGAGEKKNACSVQYRRPFGCAVLSIADLLTGETKDDLILKVYMCNTESEWYQIHENIIKKLNARYNLTGSNAGLAVSLQLLHGDIEQIRREYSSVFSHGVSITRKLGFSNIIMPGEMRNDLYITIERGEFEKGGKSVARNVEVTMFIVDSSGQTLKDFISFGSGEPPASEYHSFVLYHNNSPRWSELLKLPIPVDKFRGAHIRFEFRHCSTKEKGEKKLFGFSFVPLMQEDGRTLPDGTHELIVHKCEENTNLQDTTRYLKLPFSKGIFLGNNNQAMKATKESFCITSFLCSTKLTQNGDMLDLLKWRTHPDKITGCLSKLKEIDGSEIVKFLQDTLDTLFGILDENSQKYGSKVFDSLVHIINLLQDSKFHHFKPVMDTYIESHFAGALAYRDLIKVLKWYVDRITEAERQEHIQEVLKAQEYIFKYIVQSRRLFSLATGGQNEEEFRCCIQELLMSVRFFLSQESKGSGALSQSQAVFLSSFPAVYSELLKLFDVREVANLVQDTLGSLPTILHVDDSLQAIKLQCIGKTVESQLYTNPDSRYILLPVVLHHLHIHLQEQKDLIMCARILSNVFCLIKKNSSEKSVLEEIDVIVASLLDILLRTILEITSRPQPSSSAMRFQFQDVTGEFVACLLSLLRQMTDRHYQQLLDSFNTKEELRDFLLQIFTVFRILIRPEMFPKDWTVMRLVANNVIITTVLYLSDALRKNFLNENFDYKIWDSYFYLAVIFINQLCLQLEMFTPSKKKKVLEKYGDMRVTMGCEIFSMWQNLGEHKLHFIPALIGPFLEVTLIPQPGLRNVMIPIFHDMMDWEQRRSGNFKQVEAKLIDKLDSLMSEGKGDETYRELFNSILLKKIERETWRESGVSLIATVTRLMERLLDYRDCMKMGEVDGKKIGCTVSLLNFYKTELNKEEMYIRYIHKLYDLHLKAQNFTEAAYTLLLYDELLEWSDRPLREFLTYPMQTEWQRKEHLHLTIIQNFDRGKCWENGIILCRKIAEQYESYYDYRNLSKMRMMEASLYDKIMDQQRLEPEFFRVGFYGKKFPFFLRNKEFVCRGHDYERLEAFQQRMLNEFPHAIAMQHANQPDETIFQAEAQYLQIYAVTPIPESQEVLQREGVPDNIKSFYKVNHIWKFRYDRPFHKGTKDKENEFKSLWVERTSLYLVQSLPGISRWFEVEKREVVEMSPLENAIEVLENKNQQLKTLISQCQTRQMQNINPLTMCLNGVIDAAVNGGVSRYQEAFFVKEYILSHPEDGEKIARLRELMLEQAQILEFGLAVHEKFVPQDMRPLHKKLVDQFFVMKSSLGIQEFSACMQASPVHFPNGSPRVCRNSAPASVSPDGTRVIPRRSPLSYPAVNRYSSSSLSSQASAEVSNITGQSESSDEVFNMQPSPSTSSLSSTHSASPNVTSSAPSSARASPLLSDKHKHSRENSCLSPRERPCSAIYPTPVEASQRMLFNHIGDGALPRSDPNLSAPEKAVNPTPSSWSLDSGKEAKNMSDSGKLISPPVPPRPTQTASPARHTTSVSPSPAGRSPLKGSVQSFTPSPVEYHSPGLISNSPVLSGSYSSGISSLSRCSTSETSGFENQVNEQSAPLPVPVPVPVPSYGGEEPVRKESKTPPPYSVYERTLRRPVPLPHSLSIPVTSEPPALPPKPLAARSSHLENGARRTDPGPRPRPLPRKVSQL

SEQ ID NO: 21 - DNA, 5901 bp, Homo sapiens
atgtggatacctacggagcacgagaaatacggcgtggttattgccagtttccgaggaaccgttccatatggcctgtcattggaaattggagatacagttcagatcctggagaagtgtgatggctggtacagaggatttgccttaaaaaacccaaatatcaagggtatatttccttccagctacgttcacttgaaaaatgcctgtgtaaagaacaaaggacaatttgaaatggttattcccactgaagactctgttatcacagaaatgacatcaacattaagagactggggaaccatgtggaaacaactctatgtgcgtaatgaaggcgatctcttccaccggctgtggcacatcatgaatgaaatcctggacctgaggcggcaggtgctggtgggccacctcacccacgaccggatgaaggacgtgaagcgccacattactgcccggcttgactggggcaatgaacaactgggactggacctggtgcctaggaaagagtacgcaatggtggatccggaagacatcagcattactgagctctaccgattgatggaacatcgacatcggaagaaagacaccccggtgcaggccagcagtcaccacctctttgtccagatgaagagcctcatgtgttccaacctgggagaggagctggaggtcatcttctcactctttgacagtaaagagaaccggccaatcagtgagagatttttcttgaggctgaatagaaacgggcttcccaaagcccctgataaaccggaacgacattgctccctctttgtggatttgggcagcagtgagctaagaaaggacatttatatcaccgtgcacattatccgaatcggtcgaatgggagcaggagaaaaaaagaatgcctgtagtgtccagtaccgacgaccctttggctgtgcagttcttagcatcgctgacctgctaacaggagagacaaaggatgacctcattctgaaagtatacatgtgtaacacagagagtgagtggtaccaaatccatgagaacatcatcaaaaagctgaatgcacgttataacttgactggctccaatgcaggtttagcagtttccttacagctattgcacggagacattgaacaaatcagaagggaatattcatcagtattttctcatggagtatccataacaaggaagctgggattttcaaatattattatgcctggtgaaatgaggaatgatttatatatcactattgaaaggggagaatttgagaaaggagggaagagcgtggccagaaatgtggaagttacgatgttcattgtagacagtagtggccaaaccctgaaggattttatctccttcggctctggggagccaccagccagtgagtaccactcctttgtgctttaccataacaacagtcccaggtggtctgaactgctgaaacttcccattcctgtggataaattccggggtgcacacatccgcttcgagtttcggcattgttccacaaaggagaaaggagagaagaagttgtttgggttttcttttgtccctctgatgcaagaagatggtaggactcttccagatggcactcatgagctcatcgtgcataagtgtgaagaaaacacaaatcttcaggatactacccgctacctcaaacttcccttttccaagggcattttccttgggaataataatcaagccatgaaggccacaaaggagtccttttgtattacatcttttctctgttccacaaaactcacacaaaatggtgatatgcttgatcttttgaaatggagaacccacccagacaagatcactggctgtctctctaaattaaaagaaattgatggctcagagatagtaaagtttctgcaggatacactggataccttatttggaattttagatgaaaattcccaaaaatatgggtctaaagtgtttgattctttggttcacataataaatttgctgcaagatagcaaatttcatcattttaaacctgtaatggacacttacattgagagtcattttgctggggcacttgcatacagagatctcatcaaagtgctcaaatggtacgtggaccggatcacagaagcagagcggcaagagcatatccaggaggtgctgaaggcacaagaatacatttttaagtatatagttcaatctcgaaggctgttttcccttgccactggtgggcaaaacgaagaggagttccgctgctgcattcaggagcttctcatgtcagtccgtttctttctttcgcaagagagcaaagggtctggagcattatctcagtcacaggctgtgtttctgagctctttccctgccgtgtactcagaactgttgaagctctttgatgtccgggaagtagccaacttggtccaggacaccctgggcagtctgccgaccatcctgcatgtggatgattccctgcaggccatcaaactgcagtgcattggcaaaaccgtggaaagccagctttataccaacccagattcccgatacattcttctgcctgtcgtgttacatcacctccacattcacttgcaagaacagaaggacctgatcatgtgtgcacgtatccttagcaacgtattttgtcttatcaagaaaaatagctcagaaaaatctgtgctggaggaaatagatgtgatagtggccagcttgctggatattctgctgaggaccatattggagatcaccagccgacctcagccatccagctcagcaatgcggttccagttccaggatgtcactggggagtttgttgcttgtctcctgtccctattacgacaaatgacagatagacattatcaacagcttcttgatagttttaatacaaaggaagaactaagggatttcctgctgcagatatttactgtgttccgaatattgatacgcccggagatgtttccaaaggactggactgttatgcgcttggttgctaacaatgttattattacaacagttctatacctctcagatgcacttcgtaagaacttcttaaatgaaaactttgattataagatctgggattcctacttttacctcgcagtcatttttataaaccagttgtgtctgcagttggagatgttcacaccttccaagaagaaaaaggtgttagaaaagtatggtgacatgcgggtaacaatgggttgtgaaattttcagcatgtggcaaaacctaggagagcacaagcttcattttatccctgccctgattggccccttcctagaagtgaccttgataccccagccaggtcttcggaatgtcatgattccaatttttcatgatatgatggactgggagcagaggcggagtggcaactttaaacaggtggaagccaagctaattgacaaactggatagcctgatgtcagaaggcaaaggtgacgaaacataccgcgagctcttcaacagtattctactaaagaaaattgagcgggaaacatggcgggaaagtggcgtttcattaattgctactgtaactcgtctaatggagaggttgttagattacagggactgcatgaaaatgggagaggtagatggcaaaaagattggctgcacagttagccttctgaacttctataagactgaactgaacaaggaggagatgtatatacgctacattcacaaactctatgatctgcatctcaaagcacagaactttacagaagctgcatataccctcctcttatatgacgagctactggaatggtctgatcggcccctcagggagttcctgacctaccccatgcaaacagaatggcagcgcaaagagcacctgcacctcaccatcatccagaactttgacagaggcaaatgttgggagaatggcattatcttgtgccggaagattgcagagcagtatgagagttattatgactacagaaacctgagcaagatgcggatgatggaagcctctttgtatgacaaaattatggaccagcaacgtcttgaaccagagttcttcagagttggattttatggaaaaaaatttccatttttcttaagaaataaggagtttgtgtgtcgagggcatgactacgagaggctggaagccttccaacagagaatgctgaacgagttcccccatgccatcgccatgcagcacgccaaccagcccgatgagaccatcttccaggcagaagctcagtatttgcagatatatgctgtgactcccattccagagagccaggaggtcctgcagagagagggtgttccggacaacatcaaaagcttctataaagtgaatcacatctggaaattccgctatgaccgaccatttcacaaaggcacaaaagataaagagaatgaattcaagagtctctgggtggagagaacgtcattatacttggtgcagagtttgcctggcatctctcgctggtttgaagtggaaaagcgtgaagtggtagaaatgagtcctctggaaaatgcaattgaagtgctagaaaataagaatcagcagctgaagactctgattagtcagtgtcagacaagacagatgcagaatattaatcccctgactatgtgcctgaatggagttatagatgctgcagttaatggtggcgtttccaggtatcaagaggcattctttgtcaaagaatatatcttaagtcaccctgaagatggggagaaaattgcacgattaagagagctgatgcttgagcaggcacagattctggaatttggtttggccgtgcatgagaagtttgtacctcaagatatgagaccccttcacaaaaagctggttgaccaattctttgtgatgaagtcgagcttagggatacaggagttctctgcttgtatgcaagccagtcctgtccattttcctaatggaagccctcgtgtgtgtagaaactcagcacctgcttctgtgagcccagatggtaccagggtaattcctagacgcagcccgttaagttacccagctgtcaaccgatattcttcctcctcactgtcctcacaagcttctgctgaagtaagcaatattacagggcaatcagaaagctctgatgaagtctttaacatgcagccaagtccatctacctcaagcttgagttctactcactcggcttcacctaatgtgacaagttctgctccatcgagtgccagagcttctcctttgttgtctgacaaacacaaacattcccgagaaaactcttgcctgtcaccaagagagagaccatgcagtgccatctatccaacacctgtggagccttcgcagaggatgctgtttaatcatattggagacggggccttgccacgcagtgacccaaatctctctgcacctgaaaaagctgtgaaccccacccctagcagctggagcctggacagtgggaaggaagccaagaacatgtcggatagtgggaaacttatctctccccctgtccctccaagacccacacagactgcttcaccagcaagacacacgacatcagtatccccctcgcctgccgggcgatctccattgaagggctctgtgcagtctttcaccccctctccagtggagtaccactcgccaggactcatctccaactcccctgtcttgtcgggcagctacagcagtgggatttcttctctcagccggtgcagcacgtcggaaacctcaggctttgaaaatcaggtgaatgaacagtcggcccccctgccggtgccagtgccggtgcccgtgccgagctacggcggggaggagccagtgcgcaaggagagcaagactccgcccccgtacagcgtctacgagcggactctgcggcgccccgtcccgctacctcacagcctctccatccccgtcacgttggagccgcccgcgctgccccccaagcctctggcagcgcgatccagccacctggagaatggggcccggaggactgaccccggcccgcggcccaggcccctgccccgcaaggtctctcagttataa

SEQ ID NO: 22 - protein, 1966 aa, Homo sapiens
MWIPTEHEKYGVVIASFRGTVPYGLSLEIGDTVQILEKCDGWYRGFALKNPNIKGIFPSSYVHLKNACVKNKGQFEMVIPTEDSVITEMTSTLRDWGTMWKQLYVRNEGDLFHRLWHIMNEILDLRRQVLVGHLTHDRMKDVKRHITARLDWGNEQLGLDLVPRKEYAMVDPEDISITELYRLMEHRHRKKDTPVQASSHHLFVQMKSLMCSNLGEELEVIFSLFDSKENRPISERFFLRLNRNGLPKAPDKPERHCSLFVDLGSSELRKDIYITVHIIRIGRMGAGEKKNACSVQYRRPFGCAVLSIADLLTGETKDDLILKVYMCNTESEWYQIHENIIKKLNARYNLTGSNAGLAVSLQLLHGDIEQIRREYSSVFSHGVSITRKLGFSNIIMPGEMRNDLYITIERGEFEKGGKSVARNVEVTMFIVDSSGQTLKDFISFGSGEPPASEYHSFVLYHNNSPRWSELLKLPIPVDKFRGAHIRFEFRHCSTKEKGEKKLFGFSFVPLMQEDGRTLPDGTHELIVHKCEENTNLQDTTRYLKLPFSKGIFLGNNNQAMKATKESFCITSFLCSTKLTQNGDMLDLLKWRTHPDKITGCLSKLKEIDGSEIVKFLQDTLDTLFGILDENSQKYGSKVFDSLVHIINLLQDSKFHHFKPVMDTYIESHFAGALAYRDLIKVLKWYVDRITEAERQEHIQEVLKAQEYIFKYIVQSRRLFSLATGGQNEEEFRCCIQELLMSVRFFLSQESKGSGALSQSQAVFLSSFPAVYSELLKLFDVREVANLVQDTLGSLPTILHVDDSLQAIKLQCIGKTVESQLYTNPDSRYILLPVVLHHLHIHLQEQKDLIMCARILSNVFCLIKKNSSEKSVLEEIDVIVASLLDILLRTILEITSRPQPSSSAMRFQFQDVTGEFVACLLSLLRQMTDRHYQQLLDSFNTKEELRDFLLQIFTVFRILIRPEMFPKDWTVMRLVANNVIITTVLYLSDALRKNFLNENFDYKIWDSYFYLAVIFINQLCLQLEMFTPSKKKKVLEKYGDMRVTMGCEIFSMWQNLGEHKLHFIPALIGPFLEVTLIPQPGLRNVMIPIFHDMMDWEQRRSGNFKQVEAKLIDKLDSLMSEGKGDETYRELFNSILLKKIERETWRESGVSLIATVTRLMERLLDYRDCMKMGEVDGKKIGCTVSLLNFYKTELNKEEMYIRYIHKLYDLHLKAQNFTEAAYTLLLYDELLEWSDRPLREFLTYPMQTEWQRKEHLHLTIIQNFDRGKCWENGIILCRKIAEQYESYYDYRNLSKMRMMEASLYDKIMDQQRLEPEFFRVGFYGKKFPFFLRNKEFVCRGHDYERLEAFQQRMLNEFPHAIAMQHANQPDETIFQAEAQYLQIYAVTPIPESQEVLQREGVPDNIKSFYKVNHIWKFRYDRPFHKGTKDKENEFKSLWVERTSLYLVQSLPGISRWFEVEKREVVEMSPLENAIEVLENKNQQLKTLISQCQTRQMQNINPLTMCLNGVIDAAVNGGVSRYQEAFFVKEYILSHPEDGEKIARLRELMLEQAQILEFGLAVHEKFVPQDMRPLHKKLVDQFFVMKSSLGIQEFSACMQASPVHFPNGSPRVCRNSAPASVSPDGTRVIPRRSPLSYPAVNRYSSSSLSSQASAEVSNITGQSESSDEVFNMQPSPSTSSLSSTHSASPNVTSSAPSSARASPLLSDKHKHSRENSCLSPRERPCSAIYPTPVEPSQRMLFNHIGDGALPRSDPNLSAPEKAVNPTPSSWSLDSGKEAKNMSDSGKLISPPVPPRPTQTASPARHTTSVSPSPAGRSPLKGSVQSFTPSPVEYHSPGLISNSPVLSGSYSSGISSLSRCSTSETSGFENQVNEQSAPLPVPVPVPVPSYGGEEPVRKESKTPPPYSVYERTLRRPVPLPHSLSIPVTLEPPALPPKPLAARSSHLENGARRTDPGPRPRPLPRKVSQL

SEQ ID NO: 23 - DNA, 5901 bp, Homo sapiens
atgtggatacctacggagcacgagaaatacggcgtggttattgccagtttccgaggaaccgttccatatggcctgtcattggaaattggagatacagttcagatcctggagaagtgtgatggctggtacagaggatttgccttaaaaaacccaaatatcaagggtatatttccttccagctacgttcacttgaaaaatgcctgtgtaaagaacaaaggacaatttgaaatggttattcccactgaagactctgttatcacagaaatgacatcaacattaagagactggggaaccatgtggaaacaactctatgtgcgtaatgaaggcgatctcttccaccggctgtggcacatcatgaatgaaatcctggacctgaggcggcaggtgctggtgggccacctcacccacgaccggatgaaggacgtgaagcgccacattactgcccggcttgactggggcaatgaacaactgggactggacctggtgcctaggaaagagtacgcaatggtggatccggaagacatcagcattactgagctctaccgattgatggaacatcgacatcggaagaaagacaccccggtgcaggccagcagtcaccacctctttgtccagatgaagagcctcatgtgttccaacctgggagaggagctggaggtcatcttctcactctttgacagtaaagagaaccggccaatcagtgagagatttttcttgaggctgaatagaaacgggcttcccaaagcccctgataaaccggaacgacattgctccctctttgtggatttgggcagcagtgagctaagaaaggacatttatatcaccgtgcacattatccgaatcggtcgaatgggagcaggagaaaaaaagaatgcctgtagtgtccagtaccgacgaccctttggctgtgcagttcttagcatcgctgacctgctaacaggagagacaaaggatgacctcattctgaaagtatacatgtgtaacacagagagtgagtggtaccaaatccatgagaacatcatcaaaaagctgaatgcacgttataacttgactggctccaatgcaggtttagcagtttccttacagctattgcacggagacattgaacaaatcagaagggaatattcatcagtattttctcatggagtatccataacaaggaagctgggattttcaaatattattatgcctggtgaaatgaggaatgatttatatatcactattgaaaggggagaatttgagaaaggagggaagagcgtggccagaaatgtggaagttacgatgttcattgtagacagtagtggccaaaccctgaaggattttatctccttcggctctggggagccaccagccagtgagtaccactcctttgtgctttaccataacaacagtcccaggtggtctgaactgctgaaacttcccattcctgtggataaattccggggtgcacacatccgcttcgagtttcggcattgttccacaaaggagaaaggagagaagaagttgtttgggttttcttttgtccctctgatgcaagaagatggtaggactcttccagatggcactcatgagctcatcgtgcataagtgtgaagaaaacacaaatcttcaggatactacccgctacctcaaacttcccttttccaagggcattttccttgggaataataatcaagccatgaaggccacaaaggagtccttttgtattacatcttttctctgttccacaaaactcacacaaaatggtgatatgcttgatcttttgaaatggagaacccacccagacaagatcactggctgtctctctaaattaaaagaaattgatggctcagagatagtaaagtttctgcaggatacactggataccttatttggaattttagatgaaaattcccaaaaatatgggtctaaagtgtttgattctttggttcacataataaatttgctgcaagatagcaaatttcatcattttaaacctgtaatggacacttacattgagagtcattttgctggggcacttgcatacagagatctcatcaaagtgctcaaatggtacgtggaccggatcacagaagcagagcggcaagagcatatccaggaggtgctgaaggcacaagaatacatttttaagtatatagttcaatctcgaaggctgttttcccttgccactggtgggcaaaacgaagaggagttccgctgctgcattcaggagcttctcatgtcagtccgtttctttctttcgcaagagagcaaagggtctggagcattatctcagtcacaggctgtgtttctgagctctttccctgccgtgtactcagaactgttgaagctctttgatgtccgggaagtagccaacttggtccaggacaccctgggcagtctgccgaccatcctgcatgtggatgattccctgcaggccatcaaactgcagtgcattggcaaaaccgtggaaagccagctttataccaacccagattcccgatacattcttctgcctgtcgtgttacatcacctccacattcacttgcaagaacagaaggacctgatcatgtgtgcacgtatccttagcaacgtattttgtcttatcaagaaaaatagctcagaaaaatctgtgctggaggaaatagatgtgatagtggccagcttgctggatattctgctgaggaccatattggagatcaccagccgacctcagccatccagctcagcaatgcggttccagttccaggatgtcactggggagtttgttgcttgtctcctgtccctattacgacaaatgacagatagacattatcaacagcttcttgatagttttaatacaaaggaagaactaagggatttcctgctgcagatatttactgtgttccgaatattgatacgcccggagatgtttccaaaggactggactgttatgcgcttggttgctaacaatgttattattacaacagttctatacctctcagatgcacttcgtaagaacttcttaaatgaaaactttgattataagatctgggattcctacttttacctcgcagtcatttttataaaccagttgtgtctgcagttggagatgttcacaccttccaagaagaaaaaggtgttagaaaagtatggtgacatgcgggtaacaatgggttgtgaaattttcagcatgtggcaaaacctaggagagcacaagcttcattttatccctgccctgattggccccttcctagaagtgaccttgataccccagccaggtcttcggaatgtcatgattccaatttttcatgatatgatggactgggagcagaggcggagtggcaactttaaacaggtggaagccaagctaattgacaaactggatagcctgatgtcagaaggcaaaggtgacgaaacataccgcgagctcttcaacagtattctactaaagaaaattgagcgggaaacatggcgggaaagtggcgtttcattaattgctactgtaactcgtctaatggagaggttgttagattacagggactgcatgaaaatgggagaggtagatggcaaaaagattggctgcacagttagccttctgaacttctataagactgaactgaacaaggaggagatgtatatacgctacattcacaaactctatgatctgcatctcaaagcacagaactttacagaagctgcatataccctcctcttatatgacgagctactggaatggtctgatcggcccctcagggagttcctgacctaccccatgcaaacagaatggcagcgcaaagagcacctgcacctcaccatcatccagaactttgacagaggcaaatgttgggagaatggcattatcttgtgccggaagattgcagagcagtatgagagttattatgactacagaaacctgagcaagatgcggatgatggaagcctctttgtatgacaaaattatggaccagcaacgtcttgaaccagagttcttcagagttggattttatggaaaaaaatttccatttttcttaagaaataaggagtttgtgtgtcgagggcatgactacgagaggctggaagccttccaacagagaatgctgaacgagttcccccatgccatcgccatgcagcacgccaaccagcccgatgagaccatcttccaggcagaagctcagtatttgcagatatatgctgtgactcccattccagagagccaggaggtcctgcagagagagggtgttccggacaacatcaaaagcttctataaagtgaatcacatctggaaattccgctatgaccgaccatttcacaaaggcacaaaagataaagagaatgaattcaagagtctctgggtggagagaacgtcattatacttggtgcagagtttgcctggcatctctcgctggtttgaagtggaaaagcgtgaagtggtagaaatgagtcctctggaaaatgcaattgaagtgctagaaaataagaatcagcagctgaagactctgattagtcagtgtcagacaagacagatgcagaatattaatcccctgactatgtgcctgaatggagttatagatgctgcagttaatggtggcgtttccaggtatcaagaggcattctttgtcaaagaatatatcttaagtcaccctgaagatggggagaaaattgcacgattaagagagctgatgcttgagcaggcacagattctggaatttggtttggccgtgcatgagaagtttgtacctcaagatatgagaccccttcacaaaaagctggttgaccaattctttgtgatgaagtcgagcttagggatacaggagttctctgcttgtatgcaagccagtcctgtccattttcctaatggaagccctcgtgtgtgtagaaactcagcacctgcttctgtgagcccagatggtaccagggtaattcctagacgcagcccgttaagttacccagctgtcaaccgatattcttcctcctcactgtcctcacaagcttctgctgaagtaagcaatattacagggcaatcagaaagctctgatgaagtctttaacatgcagccaagtccatctacctcaagcttgagttctactcactcggcttcacctaatgtgacaagttctgctccatcgagtgccagagcttctcctttgttgtctgacaaacacaaacattcccgagaaaactcttgcctgtcaccaagagagagaccatgcagtgccatctatccaacacctgtggagccttcgcagaggatgctgtttaatcatattggagacggggccttgccacgcagtgacccaaatctctctgcacctgaaaaagctgtgaaccccacccctagcagctggagcctggacagtgggaaggaagccaagaacatgtcggatagtgggaaacttatctctccccctgtccctccaagacccacacagactgcttcaccagcaagacacacgacatcagtatccccctcgcctgccgggcgatctccattgaagggctctgtgcagtctttcaccccctctccagtggagtaccactcgccaggactcatctccaactcccctgtcttgtcgggcagctacagcagtgggatttcttctctcagccggtgcagcacgtcggaaacctcaggctttgaaaatcaggtgaatgaacagtcggcccccctgccggtgccagtgccggtgcccgtgccgagctacggcggggaggagccagtgcgcaaggagagcaagactccgcccccgtacagcgtctacgagcggactctgcggcgccccgtcccgctacttcacagcctctccatccccgtcacgtcggagccgcccgcgctgccccccaagcctctggcagcgcgatccagccacctggagaatggggcccggaggactgaccccggcccgcggcccaggcccctgccccgcaaggtctctcagttataa

SEQ ID NO: 24 - protein, 1966 aa, Homo sapiens
MWIPTEHEKYGVVIASFRGTVPYGLSLEIGDTVQILEKCDGWYRGFALKNPNIKGIFPSSYVHLKNACVKNKGQFEMVIPTEDSVITEMTSTLRDWGTMWKQLYVRNEGDLFHRLWHIMNEILDLRRQVLVGHLTHDRMKDVKRHITARLDWGNEQLGLDLVPRKEYAMVDPEDISITELYRLMEHRHRKKDTPVQASSHHLFVQMKSLMCSNLGEELEVIFSLFDSKENRPISERFFLRLNRNGLPKAPDKPERHCSLFVDLGSSELRKDIYITVHIIRIGRMGAGEKKNACSVQYRRPFGCAVLSIADLLTGETKDDLILKVYMCNTESEWYQIHENIIKKLNARYNLTGSNAGLAVSLQLLHGDIEQIRREYSSVFSHGVSITRKLGFSNIIMPGEMRNDLYITIERGEFEKGGKSVARNVEVTMFIVDSSGQTLKDFISFGSGEPPASEYHSFVLYHNNSPRWSELLKLPIPVDKFRGAHIRFEFRHCSTKEKGEKKLFGFSFVPLMQEDGRTLPDGTHELIVHKCEENTNLQDTTRYLKLPFSKGIFLGNNNQAMKATKESFCITSFLCSTKLTQNGDMLDLLKWRTHPDKITGCLSKLKEIDGSEIVKFLQDTLDTLFGILDENSQKYGSKVFDSLVHIINLLQDSKFHHFKPVMDTYIESHFAGALAYRDLIKVLKWYVDRITEAERQEHIQEVLKAQEYIFKYIVQSRRLFSLATGGQNEEEFRCCIQELLMSVRFFLSQESKGSGALSQSQAVFLSSFPAVYSELLKLFDVREVANLVQDTLGSLPTILHVDDSLQAIKLQCIGKTVESQLYTNPDSRYILLPVVLHHLHIHLQEQKDLIMCARILSNVFCLIKKNSSEKSVLEEIDVIVASLLDILLRTILEITSRPQPSSSAMRFQFQDVTGEFVACLLSLLRQMTDRHYQQLLDSFNTKEELRDFLLQIFTVFRILIRPEMFPKDWTVMRLVANNVIITTVLYLSDALRKNFLNENFDYKIWDSYFYLAVIFINQLCLQLEMFTPSKKKKVLEKYGDMRVTMGCEIFSMWQNLGEHKLHFIPALIGPFLEVTLIPQPGLRNVMIPIFHDMMDWEQRRSGNFKQVEAKLIDKLDSLMSEGKGDETYRELFNSILLKKIERETWRESGVSLIATVTRLMERLLDYRDCMKMGEVDGKKIGCTVSLLNFYKTELNKEEMYIRYIHKLYDLHLKAQNFTEAAYTLLLYDELLEWSDRPLREFLTYPMQTEWQRKEHLHLTIIQNFDRGKCWENGIILCRKIAEQYESYYDYRNLSKMRMMEASLYDKIMDQQRLEPEFFRVGFYGKKFPFFLRNKEFVCRGHDYERLEAFQQRMLNEFPHAIAMQHANQPDETIFQAEAQYLQIYAVTPIPESQEVLQREGVPDNIKSFYKVNHIWKFRYDRPFHKGTKDKENEFKSLWVERTSLYLVQSLPGISRWFEVEKREVVEMSPLENAIEVLENKNQQLKTLISQCQTRQMQNINPLTMCLNGVIDAAVNGGVSRYQEAFFVKEYILSHPEDGEKIARLRELMLEQAQILEFGLAVHEKFVPQDMRPLHKKLVDQFFVMKSSLGIQEFSACMQASPVHFPNGSPRVCRNSAPASVSPDGTRVIPRRSPLSYPAVNRYSSSSLSSQASAEVSNITGQSESSDEVFNMQPSPSTSSLSSTHSASPNVTSSAPSSARASPLLSDKHKHSRENSCLSPRERPCSAIYPTPVEPSQRMLFNHIGDGALPRSDPNLSAPEKAVNPTPSSWSLDSGKEAKNMSDSGKLISPPVPPRPTQTASPARHTTSVSPSPAGRSPLKGSVQSFTPSPVEYHSPGLISNSPVLSGSYSSGISSLSRCSTSETSGFENQVNEQSAPLPVPVPVPVPSYGGEEPVRKESKTPPPYSVYERTLRRPVPLLHSLSIPVTSEPPALPPKPLAARSSHLENGARRTDPGPRPRPLPRKVSQL

SEQ ID NO: 25 - DNA, 5901 bp, Homo sapiens
atgtggatacctacggagcacgagaaatacggcgtggttattgccagtttccgaggaaccgttccatatggcctgtcattggaaattggagatacagttcagatcctggagaagtgtgatggctggtacagaggatttgccttaaaaaacccaaatatcaagggtatatttccttccagctacgttcacttgaaaaatgcctgtgtaaagaacaaaggacaatttgaaatggttattcccactgaagactctgttatcacagaaatgacatcaacattaagagactggggaaccatgtggaaacaactctatgtgcgtaatgaaggcgatctcttccaccggctgtggcacatcatgaatgaaatcctggacctgaggcggcaggtgctggtgggccacctcacccacgaccggatgaaggacgtgaagcgccacattactgcccggcttgactggggcaatgaacaactgggactggacctggtgcctaggaaagagtacgcaatggtggatccggaagacatcagcattactgagctctaccgattgatggaacatcgacatcggaagaaagacaccccggtgcaggccagcagtcaccacctctttgtccagatgaagagcctcatgtgttccaacctgggagaggagctggaggtcatcttctcactctttgacagtaaagagaaccggccaatcagtgagagatttttcttgaggctgaatagaaacgggcttcccaaagcccctgataaaccggaacgacattgctccctctttgtggatttgggcagcagtgagctaagaaaggacatttatatcaccgtgcacattatccgaatcggtcgaatgggagcaggagaaaaaaagaatgcctgtagtgtccagtaccgacgaccctttggctgtgcagttcttagcatcgctgacctgctaacaggagagacaaaggatgacctcattctgaaagtatacatgtgtaacacagagagtgagtggtaccaaatccatgagaacatcatcaaaaagctgaatgcacgttataacttgactggctccaatgcaggtttagcagtttccttacagctattgcacggagacattgaacaaatcagaagggaatattcatcagtattttctcatggagtatccataacaaggaagctgggattttcaaatattattatgcctggtgaaatgaggaatgatttatatatcactattgaaaggggagaatttgagaaaggagggaagagcgtggccagaaatgtggaagttacgatgttcattgtagacagtagtggccaaaccctgaaggattttatctccttcggctctggggagccaccagccagtgagtaccactcctttgtgctttaccataacaacagtcccaggtggtctgaactgctgaaacttcccattcctgtggataaattccggggtgcacacatccgcttcgagtttcggcattgttccacaaaggagaaaggagagaagaagttgtttgggttttcttttgtccctctgatgcaagaagatggtaggactcttccagatggcactcatgagctcatcgtgcataagtgtgaagaaaacacaaatcttcaggatactacccgctacctcaaacttcccttttccaagggcattttccttgggaataataatcaagccatgaaggccacaaaggagtccttttgtattacatcttttctctgttccacaaaactcacacaaaatggtgatatgcttgatcttttgaaatggagaacccacccagacaagatcactggctgtctctctaaattaaaagaaattgatggctcagagatagtaaagtttctgcaggatacactggataccttatttggaattttagatgaaaattcccaaaaatatgggtctaaagtgtttgattctttggttcacataataaatttgctgcaagatagcaaatttcatcattttaaacctgtaatggacacttacattgagagtcattttgctggggcacttgcatacagagatctcatcaaagtgctcaaatggtacgtggaccggatcacagaagcagagcggcaagagcatatccaggaggtgctgaaggcacaagaatacatttttaagtatatagttcaatctcgaaggctgttttcccttgccactggtgggcaaaacgaagaggagttccgctgctgcattcaggagcttctcatgtcagtccgtttctttctttcgcaagagagcaaagggtctggagcattatctcagtcacaggctgtgtttctgagctctttccctgccgtgtactcagaactgttgaagctctttgatgtccgggaagtagccaacttggtccaggacaccctgggcagtctgccgaccatcctgcatgtggatgattccctgcaggccatcaaactgcagtgcattggcaaaaccgtggaaagccagctttataccaacccagattcccgatacattcttctgcctgtcgtgttacatcacctccacattcacttgcaagaacagaaggacctgatcatgtgtgcacgtatccttagcaacgtattttgtcttatcaagaaaaatagctcagaaaaatctgtgctggaggaaatagatgtgatagtggccagcttgctggatattctgctgaggaccatattggagatcaccagccgacctcagccatccagctcagcaatgcggttccagttccaggatgtcactggggagtttgttgcttgtctcctgtccctattacgacaaatgacagatagacattatcaacagcttcttgatagttttaatacaaaggaagaactaagggatttcctgctgcagatatttactgtgttccgaatattgatacgcccggagatgtttccaaaggactggactgttatgcgcttggttgctaacaatgttattattacaacagttctatacctctcagatgcacttcgtaagaacttcttaaatgaaaactttgattataagatctgggattcctacttttacctcgcagtcatttttataaaccagttgtgtctgcagttggagatgttcacaccttccaagaagaaaaaggtgttagaaaagtatggtgacatgcgggtaacaatgggttgtgaaattttcagcatgtggcaaaacctaggagagcacaagcttcattttatccctgccctgattggccccttcctagaagtgaccttgataccccagccaggtcttcggaatgtcatgattccaatttttcatgatatgatggactgggagcagaggcggagtggcaactttaaacaggtggaagccaagctaattgacaaactggatagcctgatgtcagaaggcaaaggtgacgaaacataccgcgagctcttcaacagtattctactaaagaaaattgagcgggaaacatggcgggaaagtggcgtttcattaattgctactgtaactcgtctaatggagaggttgttagattacagggactgcatgaaaatgggagaggtagatggcaaaaagattggctgcacagttagccttctgaacttctataagactgaactgaacaaggaggagatgtatatacgctacattcacaaactctatgatctgcatctcaaagcacagaactttacagaagctgcatataccctcctcttatatgacgagctactggaatggtctgatcggcccctcagggagttcctgacctaccccatgcaaacagaatggcagcgcaaagagcacctgcacctcaccatcatccagaactttgacagaggcaaatgttgggagaatggcattatcttgtgccggaagattgcagagcagtatgagagttattatgactacagaaacctgagcaagatgcggatgatggaagcctctttgtatgacaaaattatggaccagcaacgtcttgaaccagagttcttcagagttggattttatggaaaaaaatttccatttttcttaagaaataaggagtttgtgtgtcgagggcatgactacgagaggctggaagccttccaacagagaatgctgaacgagttcccccatgccatcgccatgcagcacgccaaccagcccgatgagaccatcttccaggcagaagctcagtatttgcagatatatgctgtgactcccattccagagagccaggaggtcctgcagagagagggtgttccggacaacatcaaaagcttctataaagtgaatcacatctggaaattccgctatgaccgaccatttcacaaaggcacaaaagataaagagaatgaattcaagagtctctgggtggagagaacgtcattatacttggtgcagagtttgcctggcatctctcgctggtttgaagtggaaaagcgtgaagtggtagaaatgagtcctctggaaaatgcaattgaagtgctagaaaataagaatcagcagctgaagactctgattagtcagtgtcagacaagacagatgcagaatattaatcccctgactatgtgcctgaatggagttatagatgctgcagttaatggtggcgtttccaggtatcaagaggcattctttgtcaaagaatatatcttaagtcaccctgaagatggggagaaaattgcacgattaagagagctgatgcttgagcaggcacagattctggaatttggtttggccgtgcatgagaagtttgtacctcaagatatgagaccccttcacaaaaagctggttgaccaattctttgtgatgaagtcgagcttagggatacaggagttctctgcttgtatgcaagccagtcctgtccattttcctaatggaagccctcgtgtgtgtagaaactcagcacctgcttctgtgagcccagatggtaccagggtaattcctagacgcagcccgttaagttacccagctgtcaaccgatattcttcctcctcactgtcctcacaagcttctgctgaagtaagcaatattacagggcaatcagaaagctctgatgaagtctttaacatgcagccaagtccatctacctcaagcttgagttctactcactcggcttcacctaatgtgacaagttctgctccatcgagtgccagagcttctcctttgttgtctgacaaacacaaacattcccgagaaaactcttgcctgtcaccaagagagagaccatgcagtgccatctatccaacacctgtggagccttcgcagaggatgctgtttaatcatattggagacggggccttgccacgcagtgacccaaatctctctgcacctgaaaaagctgtgaaccccacccctagcagctggagcctggacagtgggaaggaagccaagaacatgtcggatagtgggaaacttatctctccccctgtccctccaagacccacacagactgcttcaccagcaagacacacgacatcagtatccccctcgcctgccgggcgatctccattgaagggctctgtgcagtctttcaccccctctccagtggagtaccactcgccaggactcatctccaactcccctgtcttgtcgggcagctacagcagtgggatttcttctctcagccggtgcagcacgtcggaaacctcaggctttgaaaatcaggtgaatgaacagtcggcccccctgccggtgccagtgccggtgcccgtgccgagctacggcggggaggagccagtgcgcaaggagagcaagactccgcccccgtacagcgtctacgagcggactctgcggcgccccatcccgctacctcacagcctctccatccccgtcacgtcggagccgcccgcgctgccccccaagcctctggcagcgcgatccagccacctggagaatggggcccggaggactgaccccggcccgcggcccaggcccctgccccgcaaggtctctcagttataa

SEQ ID NO: 26 - protein, 1966 aa, Homo sapiens
MWIPTEHEKYGVVIASFRGTVPYGLSLEIGDTVQILEKCDGWYRGFALKNPNIKGIFPSSYVHLKNACVKNKGQFEMVIPTEDSVITEMTSTLRDWGTMWKQLYVRNEGDLFHRLWHIMNEILDLRRQVLVGHLTHDRMKDVKRHITARLDWGNEQLGLDLVPRKEYAMVDPEDISITELYRLMEHRHRKKDTPVQASSHHLFVQMKSLMCSNLGEELEVIFSLFDSKENRPISERFFLRLNRNGLPKAPDKPERHCSLFVDLGSSELRKDIYITVHIIRIGRMGAGEKKNACSVQYRRPFGCAVLSIADLLTGETKDDLILKVYMCNTESEWYQIHENIIKKLNARYNLTGSNAGLAVSLQLLHGDIEQIRREYSSVFSHGVSITRKLGFSNIIMPGEMRNDLYITIERGEFEKGGKSVARNVEVTMFIVDSSGQTLKDFISFGSGEPPASEYHSFVLYHNNSPRWSELLKLPIPVDKFRGAHIRFEFRHCSTKEKGEKKLFGFSFVPLMQEDGRTLPDGTHELIVHKCEENTNLQDTTRYLKLPFSKGIFLGNNNQAMKATKESFCITSFLCSTKLTQNGDMLDLLKWRTHPDKITGCLSKLKEIDGSEIVKFLQDTLDTLFGILDENSQKYGSKVFDSLVHIINLLQDSKFHHFKPVMDTYIESHFAGALAYRDLIKVLKWYVDRITEAERQEHIQEVLKAQEYIFKYIVQSRRLFSLATGGQNEEEFRCCIQELLMSVRFFLSQESKGSGALSQSQAVFLSSFPAVYSELLKLFDVREVANLVQDTLGSLPTILHVDDSLQAIKLQCIGKTVESQLYTNPDSRYILLPVVLHHLHIHLQEQKDLIMCARILSNVFCLIKKNSSEKSVLEEIDVIVASLLDILLRTILEITSRPQPSSSAMRFQFQDVTGEFVACLLSLLRQMTDRHYQQLLDSFNTKEELRDFLLQIFTVFRILIRPEMFPKDWTVMRLVANNVIITTVLYLSDALRKNFLNENFDYKIWDSYFYLAVIFINQLCLQLEMFTPSKKKKVLEKYGDMRVTMGCEIFSMWQNLGEHKLHFIPALIGPFLEVTLIPQPGLRNVMIPIFHDMMDWEQRRSGNFKQVEAKLIDKLDSLMSEGKGDETYRELFNSILLKKIERETWRESGVSLIATVTRLMERLLDYRDCMKMGEVDGKKIGCTVSLLNFYKTELNKEEMYIRYIHKLYDLHLKAQNFTEAAYTLLLYDELLEWSDRPLREFLTYPMQTEWQRKEHLHLTIIQNFDRGKCWENGIILCRKIAEQYESYYDYRNLSKMRMMEASLYDKIMDQQRLEPEFFRVGFYGKKFPFFLRNKEFVCRGHDYERLEAFQQRMLNEFPHAIAMQHANQPDETIFQAEAQYLQIYAVTPIPESQEVLQREGVPDNIKSFYKVNHIWKFRYDRPFHKGTKDKENEFKSLWVERTSLYLVQSLPGISRWFEVEKREVVEMSPLENAIEVLENKNQQLKTLISQCQTRQMQNINPLTMCLNGVIDAAVNGGVSRYQEAFFVKEYILSHPEDGEKIARLRELMLEQAQILEFGLAVHEKFVPQDMRPLHKKLVDQFFVMKSSLGIQEFSACMQASPVHFPNGSPRVCRNSAPASVSPDGTRVIPRRSPLSYPAVNRYSSSSLSSQASAEVSNITGQSESSDEVFNMQPSPSTSSLSSTHSASPNVTSSAPSSARASPLLSDKHKHSRENSCLSPRERPCSAIYPTPVEPSQRMLFNHIGDGALPRSDPNLSAPEKAVNPTPSSWSLDSGKEAKNMSDSGKLISPPVPPRPTQTASPARHTTSVSPSPAGRSPLKGSVQSFTPSPVEYHSPGLISNSPVLSGSYSSGISSLSRCSTSETSGFENQVNEQSAPLPVPVPVPVPSYGGEEPVRKESKTPPPYSVYERTLRRPIPLPHSLSIPVTSEPPALPPKPLAARSSHLENGARRTDPGPRPRPLPRKVSQL

SEQ ID NO: 27 - DNA, 5901 bp, Homo sapiens
atgtggatacctacggagcacgagaaatacggcgtggttattgccagtttccgaggaaccgttccatatggcctgtcattggaaattggagatacagttcagatcctggagaagtgtgatggctggtacagaggatttgccttaaaaaacccaaatatcaagggtatatttccttccagctacgttcacttgaaaaatgcctgtgtaaagaacaaaggacaatttgaaatggttattcccactgaagactctgttatcacagaaatgacatcaacattaagagactggggaaccatgtggaaacaactctatgtgcgtaatgaaggcgatctcttccaccggctgtggcacatcatgaatgaaatcctggacctgaggcggcaggtgctggtgggccacctcacccacgaccggatgaaggacgtgaagcgccacattactgcccggcttgactggggcaatgaacaactgggactggacctggtgcctaggaaagagtacgcaatggtggatccggaagacatcagcattactgagctctaccgattgatggaacatcgacatcggaagaaagacaccccggtgcaggccagcagtcaccacctctttgtccagatgaagagcctcatgtgttccaacctgggagaggagctggaggtcatcttctcactctttgacagtaaagagaaccggccaatcagtgagagatttttcttgaggctgaatagaaacgggcttcccaaagcccctgataaaccggaacgacattgctccctctttgtggatttgggcagcagtgagctaagaaaggacatttatatcaccgtgcacattatccgaatcggtcgaatgggagcaggagaaaaaaagaatgcctgtagtgtccagtaccgacgaccctttggctgtgcagttcttagcatcgctgacctgctaacaggagagacaaaggatgacctcattctgaaagtatacatgtgtaacacagagagtgagtggtaccaaatccatgagaacatcatcaaaaagctgaatgcacgttataacttgactggctccaatgcaggtttagcagtttccttacagctattgcacggagacattgaacaaatcagaagggaatattcatcagtattttctcatggagtatccataacaaggaagctgggattttcaaatattattatgcctggtgaaatgaggaatgatttatatatcactattgaaaggggagaatttgagaaaggagggaagagcgtggccagaaatgtggaagttacgatgttcattgtagacagtagtggccaaaccctgaaggattttatctccttcggctctggggagccaccagccagtgagtaccactcctttgtgctttaccataacaacagtcccaggtggtctgaactgctgaaacttcccattcctgtggataaattccggggtgcacacatccgcttcgagtttcggcattgttccacaaaggagaaaggagagaagaagttgtttgggttttcttttgtccctctgatgcaagaagatggtaggactcttccagatggcactcatgagctcatcgtgcataagtgtgaagaaaacacaaatcttcaggatactacccgctacctcaaacttcccttttccaagggcattttccttgggaataataatcaagccatgaaggccacaaaggagtccttttgtattacatcttttctctgttccacaaaactcacacaaaatggtgatatgcttgatcttttgaaatggagaacccacccagacaagatcactggctgtctctctaaattaaaacaaattgatggctcagagatagtaaagtttctgcaggatacactggataccttatttggaattttagatgaaaattcccaaaaatatgggtctaaagtgtttgattctttggttcacataataaatttgctgcaagatagcaaatttcatcattttaaacctgtaatggacacttacattgagagtcattttgctggggcacttgcatacagagatctcatcaaagtgctcaaatggtacgtggaccggatcacagaagcagagcggcaagagcatatccaggaggtgctgaaggcacaagaatacatttttaagtatatagttcaatctcgaaggctgttttcccttgccactggtgggcaaaacgaagaggagttccgctgctgcattcaggagcttctcatgtcagtccgtttctttctttcgcaagagagcaaagggtctggagcattatctcagtcacaggctgtgtttctgagctctttccctgccgtgtactcagaactgttgaagctctttgatgtccgggaagtagccaacttggtccaggacaccctgggcagtctgccgaccatcctgcatgtggatgattccctgcaggccatcaaactgcagtgcattggcaaaaccgtggaaagccagctttataccaacccagattcccgatacattcttctgcctgtcgtgttacatcacctccacattcacttgcaagaacagaaggacctgatcatgtgtgcacgtatccttagcaacgtattttgtcttatcaagaaaaatagctcagaaaaatctgtgctggaggaaatagatgtgatagtggccagcttgctggatattctgctgaggaccatattggagatcaccagccgacctcagccatccagctcagcaatgcggttccagttccaggatgtcactggggagtttgttgcttgtctcctgtccctattacgacaaatgacagatagacattatcaacagcttcttgatagttttaatacaaaggaagaactaagggatttcctgctgcagatatttactgtgttccgaatattgatacgcccggagatgtttccaaaggactggactgttatgcgcttggttgctaacaatgttattattacaacagttctatacctctcagatgcacttcgtaagaacttcttaaatgaaaactttgattataagatctgggattcctacttttacctcgcagtcatttttataaaccagttgtgtctgcagttggagatgttcacaccttccaagaagaaaaaggtgttagaaaagtatggtgacatgcgggtaacaatgggttgtgaaattttcagcatgtggcaaaacctaggagagcacaagcttcattttatccctgccctgattggccccttcctagaagtgaccttgataccccagccaggtcttcggaatgtcatgattccaatttttcatgatatgatggactgggagcagaggcggagtggcaactttaaacaggtggaagccaagctaattgacaaactggatagcctgatgtcagaaggcaaaggtgacgaaacataccgcgagctcttcaacagtattctactaaagaaaattgagcgggaaacatggcgggaaagtggcgtttcattaattgctactgtaactcgtctaatggagaggttgttagattacagggactgcatgaaaatgggagaggtagatggcaaaaagattggctgcacagttagccttctgaacttctataagactgaactgaacaaggaggagatgtatatacgctacattcacaaactctatgatctgcatctcaaagcacagaactttacagaagctgcatataccctcctcttatatgacgagctactggaatggtctgatcggcccctcagggagttcctgacctaccccatgcaaacagaatggcagcgcaaagagcacctgcacctcaccatcatccagaactttgacagaggcaaatgttgggagaatggcattatcttgtgccggaagattgcagagcagtatgagagttattatgactacagaaacctgagcaagatgcggatgatggaagcctctttgtatgacaaaattatggaccagcaacgtcttgaaccagagttcttcagagttggattttatggaaaaaaatttccatttttcttaagaaataaggagtttgtgtgtcgagggcatgactacgagaggctggaagccttccaacagagaatgctgaacgagttcccccatgccatcgccatgcagcacgccaaccagcccgatgagaccatcttccaggcagaagctcagtatttgcagatatatgctgtgactcccattccagagagccaggaggtcctgcagagagagggtgttccggacaacatcaaaagcttctataaagtgaatcacatctggaaattccgctatgaccgaccatttcacaaaggcacaaaagataaagagaatgaattcaagagtctctgggtggagagaacgtcattatacttggtgcagagtttgcctggcatctctcgctggtttgaagtggaaaagcgtgaagtggtagaaatgagtcctctggaaaatgcaattgaagtgctagaaaataagaatcagcagctgaagactctgattagtcagtgtcagacaagacagatgcagaatattaatcccctgactatgtgcctgaatggagttatagatgctgcagttaatggtggcgtttccaggtatcaagaggcattctttgtcaaagaatatatcttaagtcaccctgaagatggggagaaaattgcacgattaagagagctgatgcttgagcaggcacagattctggaatttggtttggccgtgcatgagaagtttgtacctcaagatatgagaccccttcacaaaaagctggttgaccaattctttgtgatgaagtcgagcttagggatacaggagttctctgcttgtatgcaagccagtcctgtccattttcctaatggaagccctcgtgtgtgtagaaactcagcacctgcttctgtgagcccagatggtaccagggtaattcctagacgcagcccgttaagttacccagctgtcaaccgatattcttcctcctcactgtcctcacaagcttctgctgaagtaagcaatattacagggcaatcagaaagctctgatgaagtctttaacatgcagccaagtccatctacctcaagcttgagttctactcactcggcttcacctaatgtgacaagttctgctccatcgagtgccagagcttctcctttgttgtctgacaaacacaaacattcccgagaaaactcttgcctgtcaccaagagagagaccatgcagtgccatctatccaacacctgtggagccttcgcagaggatgctgtttaatcatattggagacggggccttgccacgcagtgacccaaatctctctgcacctgaaaaagctgtgaaccccacccctagcagctggagcctggacagtgggaaggaagccaagaacatgtcggatagtgggaaacttatctctccccctgtccctccaagacccacacagactgcttcaccagcaagacacacgacatcagtatccccctcgcctgccgggcgatctccattgaagggctctgtgcagtctttcaccccctctccagtggagtaccactcgccaggactcatctccaactcccctgtcttgtcgggcagctacagcagtgggatttcttctctcagccggtgcagcacgtcggaaacctcaggctttgaaaatcaggtgaatgaacagtcggcccccctgccggtgccagtgccggtgcccgtgccgagctacggcggggaggagccagtgcgcaaggagagcaagactccgcccccgtacagcgtctacgagcggactctgcggcgccccgtcccgctacctcacagcctctccatccccgtcacgtcggagccgcccgcgctgccccccaagcctctggcagcgcgatccagccacctggagaatggggcccggaggactgaccccggcccgcggcccaggcccctgccccgcaaggtctctcagttataa

SEQ ID NO: 28 - protein, 1966 aa, Homo sapiens
MWIPTEHEKYGVVIASFRGTVPYGLSLEIGDTVQILEKCDGWYRGFALKNPNIKGIFPSSYVHLKNACVKNKGQFEMVIPTEDSVITEMTSTLRDWGTMWKQLYVRNEGDLFHRLWHIMNEILDLRRQVLVGHLTHDRMKDVKRHITARLDWGNEQLGLDLVPRKEYAMVDPEDISITELYRLMEHRHRKKDTPVQASSHHLFVQMKSLMCSNLGEELEVIFSLFDSKENRPISERFFLRLNRNGLPKAPDKPERHCSLFVDLGSSELRKDIYITVHIIRIGRMGAGEKKNACSVQYRRPFGCAVLSIADLLTGETKDDLILKVYMCNTESEWYQIHENIIKKLNARYNLTGSNAGLAVSLQLLHGDIEQIRREYSSVFSHGVSITRKLGFSNIIMPGEMRNDLYITIERGEFEKGGKSVARNVEVTMFIVDSSGQTLKDFISFGSGEPPASEYHSFVLYHNNSPRWSELLKLPIPVDKFRGAHIRFEFRHCSTKEKGEKKLFGFSFVPLMQEDGRTLPDGTHELIVHKCEENTNLQDTTRYLKLPFSKGIFLGNNNQAMKATKESFCITSFLCSTKLTQNGDMLDLLKWRTHPDKITGCLSKLKQIDGSEIVKFLQDTLDTLFGILDENSQKYGSKVFDSLVHIINLLQDSKFHHFKPVMDTYIESHFAGALAYRDLIKVLKWYVDRITEAERQEHIQEVLKAQEYIFKYIVQSRRLFSLATGGQNEEEFRCCIQELLMSVRFFLSQESKGSGALSQSQAVFLSSFPAVYSELLKLFDVREVANLVQDTLGSLPTILHVDDSLQAIKLQCIGKTVESQLYTNPDSRYILLPVVLHHLHIHLQEQKDLIMCARILSNVFCLIKKNSSEKSVLEEIDVIVASLLDILLRTILEITSRPQPSSSAMRFQFQDVTGEFVACLLSLLRQMTDRHYQQLLDSFNTKEELRDFLLQIFTVFRILIRPEMFPKDWTVMRLVANNVIITTVLYLSDALRKNFLNENFDYKIWDSYFYLAVIFINQLCLQLEMFTPSKKKKVLEKYGDMRVTMGCEIFSMWQNLGEHKLHFIPALIGPFLEVTLIPQPGLRNVMIPIFHDMMDWEQRRSGNFKQVEAKLIDKLDSLMSEGKGDETYRELFNSILLKKIERETWRESGVSLIATVTRLMERLLDYRDCMKMGEVDGKKIGCTVSLLNFYKTELNKEEMYIRYIHKLYDLHLKAQNFTEAAYTLLLYDELLEWSDRPLREFLTYPMQTEWQRKEHLHLTIIQNFDRGKCWENGIILCRKIAEQYESYYDYRNLSKMRMMEASLYDKIMDQQRLEPEFFRVGFYGKKFPFFLRNKEFVCRGHDYERLEAFQQRMLNEFPHAIAMQHANQPDETIFQAEAQYLQIYAVTPIPESQEVLQREGVPDNIKSFYKVNHIWKFRYDRPFHKGTKDKENEFKSLWVERTSLYLVQSLPGISRWFEVEKREVVEMSPLENAIEVLENKNQQLKTLISQCQTRQMQNINPLTMCLNGVIDAAVNGGVSRYQEAFFVKEYILSHPEDGEKIARLRELMLEQAQILEFGLAVHEKFVPQDMRPLHKKLVDQFFVMKSSLGIQEFSACMQASPVHFPNGSPRVCRNSAPASVSPDGTRVIPRRSPLSYPAVNRYSSSSLSSQASAEVSNITGQSESSDEVFNMQPSPSTSSLSSTHSASPNVTSSAPSSARASPLLSDKHKHSRENSCLSPRERPCSAIYPTPVEPSQRMLFNHIGDGALPRSDPNLSAPEKAVNPTPSSWSLDSGKEAKNMSDSGKLISPPVPPRPTQTASPARHTTSVSPSPAGRSPLKGSVQSFTPSPVEYHSPGLISNSPVLSGSYSSGISSLSRCSTSETSGFENQVNEQSAPLPVPVPVPVPSYGGEEPVRKESKTPPPYSVYERTLRRPVPLPHSLSIPVTSEPPALPPKPLAARSSHLENGARRTDPGPRPRPLPRKVSQL

SEQ ID NO: 29 - DNA, 5787 bp, Homo sapiens
atgtggatacctacggagcacgagaaatacggcgtggttattgccagtttccgaggaaccgttccatatggcctgtcattggaaattggagatacagttcagatcctggagaagtgtgatggctggtacagaggatttgccttaaaaaacccaaatatcaagggtatatttccttccagctacgttcacttgaaaaatgcctgtgtaaagaacaaaggacaatttgaaatggttattcccactgaagactctgttatcacagaaatgacatcaacattaagagactggggaaccatgtggaaacaactctatgtgcgtaatgaaggcgatctcttccaccggctgtggcacatcatgaatgaaatcctggacctgaggcggcaggtgctggtgggccacctcacccacgaccggatgaaggacgtgaagcgccacattactgcccggcttgactggggcaatgaacaactgggactggacctggtgcctaggaaagagtacgcaatggtggatccggaagacatcagcattactgagctctaccgattgatggaacatcgacatcggaagaaagacaccccggtgcaggccagcagtcaccacctctttgtccagatgaagagcctcatgtgttccaacctgggagaggagctggaggtcatcttctcactctttgacagtaaagagaaccggccaatcagtgagagatttttcttgaggctgaatagaaacgggcttcccaaagcccctgataaaccggaacgacattgctccctctttgtggatttgggcagcagtgagctaagaaaggacatttatatcaccgtgcacattatccgaatcggtcgaatgggagcaggagaaaaaaagaatgcctgtagtgtccagtaccgacgaccctttggctgtgcagttcttagcatcgctgacctgctaacaggagagacaaaggatgacctcattctgaaagtatacatgtgtaacacagagagtgagtggtaccaaatccatgagaacatcatcaaaaagctgaatgcacgttataacttgactggctccaatgcaggtttagcagtttccttacagctattgcacggagacattgaacaaatcagaagggaatattcatcagtattttctcatggagtatccataacaaggaagctgggattttcaaatattattatgcctggtgaaatgaggaatgatttatatatcactattgaaaggggagaatttgagaaaggagggaagagcgtggccagaaatgtggaagttacgatgttcattgtagacagtagtggccaaaccctgaaggattttatctccttcggctctggggagccaccagccagtgagtaccactcctttgtgctttaccataacaacagtcccaggtggtctgaactgctgaaacttcccattcctgtggataaattccggggtgcacacatccgcttcgagtttcggcattgttccacaaaggagaaaggagagaagaagttgtttgggttttcttttgtccctctgatgcaagaagatggtaggactcttccagatggcactcatgagctcatcgtgcataagtgtgaagaaaacacaaatcttcaggatactacccgctacctcaaacttcccttttccaagggcattttccttgggaataataatcaagccatgaaggccacaaaggagtccttttgtattacatcttttctctgttccacaaaactcacacaaaatggtgatatgcttgatcttttgaaatggagaacccacccagacaagatcactggctgtctctctaaattaaaagaaattgatggctcagagatagtaaagtttctgcaggatacactggataccttatttggaattttagatgaaaattcccaaaaatatgggtctaaagtgtttgattctttggttcacataataaatttgctgcaagatagcaaatttcatcattttaaacctgtaatggacacttacattgagagtcattttgctggggcacttgcatacagagatctcatcaaagtgctcaaatggtacgtggaccggatcacagaagcagagcggcaagagcatatccaggaggtgctgaaggcacaagaatacatttttaagtatatagttcaatctcgaaggctgttttcccttgccactggtgggcaaaacgaagaggagttccgctgctgcattcaggagcttctcatgtcagtccgtttctttctttcgcaagagagcaaagggtctggagcattatctcagtcacaggctgtgtttctgagctctttccctgccgtgtactcagaactgttgaagctctttgatgtccgggaagtagccaacttggtccaggacaccctgggcagtctgccgaccatcctgcatgtggatgattccctgcaggccatcaaactgcagtgcattggcaaaaccgtggaaagccagctttataccaacccagattcccgatacattcttctgcctgtcgtgttacatcacctccacattcacttgcaagaacagaaggacctgatcatgtgtgcacgtatccttagcaacgtattttgtcttatcaagaaaaatagctcagaaaaatctgtgctggaggaaatagatgtgatagtggccagcttgctggatattctgctgaggaccatattggagatcaccagccgacctcagccatccagctcagcaatgcggttccagttccaggatgtcactggggagtttgttgcttgtctcctgtccctattacgacaaatgacagatagacattatcaacagcttcttgatagttttaatacaaaggaagaactaagggatttcctgctgcagatatttactgtgttccgaatattgatacgcccggagatgtttccaaaggactggactgttatgcgcttggttgctaacaatgttattattacaacagttctatacctctcagatgcacttcgtaagaacttcttaaatgaaaactttgattataagatctgggattcctacttttacctcgcagtcatttttataaaccagttgtgtctgcagttggagatgttcacaccttccaagaagaaaaaggtgttagaaaagtatggtgacatgcgggtaacaatgggttgtgaaattttcagcatgtggcaaaacctaggagagcacaagcttcattttatccctgccctgattggccccttcctagaagtgaccttgataccccagccaggtcttcggaatgtcatgattccaatttttcatgatatgatggactgggagcagaggcggagtggcaactttaaacaggtggaagccaagctaattgacaaactggatagcctgatgtcagaaggcaaaggtgacgaaacataccgcgagctcttcaacagtattctactaaagaaaattgagcgggaaacatggcgggaaagtggcgtttcattaattgctactgtaactcgtctaatggagaggttgttagattacagggactgcatgaaaatgggagaggtagatggcaaaaagattggctgcacagttagccttctgaacttctataagactgaactgaacaaggaggagatgtatatacgctacattcacaaactctatgatctgcatctcaaagcacagaactttacagaagctgcatataccctcctcttatatgacgagctactggaatggtctgatcggcccctcagggagttcctgacctaccccatgcaaacagaatggcagcgcaaagagcacctgcacctcaccatcatccagaactttgacagaggcaaatgttgggagaatggcattatcttgtgccggaagattgcagagcagtatgagagttattatgactacagaaacctgagcaagatgcggatgatggaagcctctttgtatgacaaaattatggaccagcaacgtcttgaaccagagttcttcagagttggattttatggaaaaaaatttccatttttcttaagaaataaggagtttgtgtgtcgagggcatgactacgagaggctggaagccttccaacagagaatgctgaacgagttcccccatgccatcgccatgcagcacgccaaccagcccgatgagaccatcttccaggcagaagctcagtatttgcagatatatgctgtgactcccattccagagagccaggaggtcctgcagagagagggtgttccggacaacatcaaaagcttctataaagtgaatcacatctggaaattccgctatgaccgaccatttcacaaaggcacaaaagataaagagaatgaattcaagagtctctgggtggagagaacgtcattatacttggtgcagagtttgcctggcatctctcgctggtttgaagtggaaaagcgtgaagtggtagaaatgagtcctctggaaaatgcaattgaagtgctagaaaataagaatcagcagctgaagactctgattagtcagtgtcagacaagacagatgcagaatattaatcccctgactatgtgcctgaatggagttatagatgctgcagttaatggtggcgtttccaggtatcaagaggcattctttgtcaaagaatatatcttaagtcaccctgaagatggggagaaaattgcacgattaagagagctgatgcttgagcaggcacagattctggaatttggtttggccgtgcatgagaagtttgtacctcaagatatgagaccccttcacaaaaagctggttgaccaattctttgtgatgaagtcgagcttagggatacaggagttctctgcttgtatgcaagccagtcctgtccattttcctaatggaagccctcgtgtgtgtagaaactcagcacctgcttctgtgagcccagatggtaccagggtaattcctagacgcagcccgttaagttacccagctgtcaaccgatattcttcctcctcactgtcctcacaagcttctgctgaagtaagcaatattacagggcaatcagaaagctctgatgaagtctttaacatgcagccaagtccatctacctcaagcttgagttctactcactcggcttcacctaatgtgacaagttctgctccatcgagtgccagagcttctcctttgttgtctgacaaacacaaacattcccgagaaaactcttgcctgtcaccaagagagagaccatgcagtgccatctatccaacacctgtggagccttcgcagaggatgctgtttaatcatattggagacggggccttgccacgcagtgacccaaatctctctgcacctgaaaaagcttcaccagcaagacacacgacatcagtatccccctcgcctgccgggcgatctccattgaagggctctgtgcagtctttcaccccctctccagtggagtaccactcgccaggactcatctccaactcccctgtcttgtcgggcagctacagcagtgggatttcttctctcagccggtgcagcacgtcggaaacctcaggctttgaaaatcaggtgaatgaacagtcggcccccctgccggtgccagtgccggtgcccgtgccgagctacggcggggaggagccagtgcgcaaggagagcaagactccgcccccgtacagcgtctacgagcggactctgcggcgccccgtcccgctacctcacagcctctccatccccgtcacgtcggagccgcccgcgctgccccccaagcctctggcagcgcgatccagccacctggagaatggggcccggaggactgaccccggcccgcggcccaggcccctgccccgcaaggtctctcagttataa

SEQ ID NO: 30 - protein, 1928 aa, Homo sapiens
MWIPTEHEKYGVVIASFRGTVPYGLSLEIGDTVQILEKCDGWYRGFALKNPNIKGIFPSSYVHLKNACVKNKGQFEMVIPTEDSVITEMTSTLRDWGTMWKQLYVRNEGDLFHRLWHIMNEILDLRRQVLVGHLTHDRMKDVKRHITARLDWGNEQLGLDLVPRKEYAMVDPEDISITELYRLMEHRHRKKDTPVQASSHHLFVQMKSLMCSNLGEELEVIFSLFDSKENRPISERFFLRLNRNGLPKAPDKPERHCSLFVDLGSSELRKDIYITVHIIRIGRMGAGEKKNACSVQYRRPFGCAVLSIADLLTGETKDDLILKVYMCNTESEWYQIHENIIKKLNARYNLTGSNAGLAVSLQLLHGDIEQIRREYSSVFSHGVSITRKLGFSNIIMPGEMRNDLYITIERGEFEKGGKSVARNVEVTMFIVDSSGQTLKDFISFGSGEPPASEYHSFVLYHNNSPRWSELLKLPIPVDKFRGAHIRFEFRHCSTKEKGEKKLFGFSFVPLMQEDGRTLPDGTHELIVHKCEENTNLQDTTRYLKLPFSKGIFLGNNNQAMKATKESFCITSFLCSTKLTQNGDMLDLLKWRTHPDKITGCLSKLKEIDGSEIVKFLQDTLDTLFGILDENSQKYGSKVFDSLVHIINLLQDSKFHHFKPVMDTYIESHFAGALAYRDLIKVLKWYVDRITEAERQEHIQEVLKAQEYIFKYIVQSRRLFSLATGGQNEEEFRCCIQELLMSVRFFLSQESKGSGALSQSQAVFLSSFPAVYSELLKLFDVREVANLVQDTLGSLPTILHVDDSLQAIKLQCIGKTVESQLYTNPDSRYILLPVVLHHLHIHLQEQKDLIMCARILSNVFCLIKKNSSEKSVLEEIDVIVASLLDILLRTILEITSRPQPSSSAMRFQFQDVTGEFVACLLSLLRQMTDRHYQQLLDSFNTKEELRDFLLQIFTVFRILIRPEMFPKDWTVMRLVANNVIITTVLYLSDALRKNFLNENFDYKIWDSYFYLAVIFINQLCLQLEMFTPSKKKKVLEKYGDMRVTMGCEIFSMWQNLGEHKLHFIPALIGPFLEVTLIPQPGLRNVMIPIFHDMMDWEQRRSGNFKQVEAKLIDKLDSLMSEGKGDETYRELFNSILLKKIERETWRESGVSLIATVTRLMERLLDYRDCMKMGEVDGKKIGCTVSLLNFYKTELNKEEMYIRYIHKLYDLHLKAQNFTEAAYTLLLYDELLEWSDRPLREFLTYPMQTEWQRKEHLHLTIIQNFDRGKCWENGIILCRKIAEQYESYYDYRNLSKMRMMEASLYDKIMDQQRLEPEFFRVGFYGKKFPFFLRNKEFVCRGHDYERLEAFQQRMLNEFPHAIAMQHANQPDETIFQAEAQYLQIYAVTPIPESQEVLQREGVPDNIKSFYKVNHIWKFRYDRPFHKGTKDKENEFKSLWVERTSLYLVQSLPGISRWFEVEKREVVEMSPLENAIEVLENKNQQLKTLISQCQTRQMQNINPLTMCLNGVIDAAVNGGVSRYQEAFFVKEYILSHPEDGEKIARLRELMLEQAQILEFGLAVHEKFVPQDMRPLHKKLVDQFFVMKSSLGIQEFSACMQASPVHFPNGSPRVCRNSAPASVSPDGTRVIPRRSPLSYPAVNRYSSSSLSSQASAEVSNITGQSESSDEVFNMQPSPSTSSLSSTHSASPNVTSSAPSSARASPLLSDKHKHSRENSCLSPRERPCSAIYPTPVEPSQRMLFNHIGDGALPRSDPNLSAPEKASPARHTTSVSPSPAGRSPLKGSVQSFTPSPVEYHSPGLISNSPVLSGSYSSGISSLSRCSTSETSGFENQVNEQSAPLPVPVPVPVPSYGGEEPVRKESKTPPPYSVYERTLRRPVPLPHSLSIPVTSEPPALPPKPLAARSSHLENGARRTDPGPRPRPLPRKVSQL

SEQ ID NO: 31 - DNA, 114 bp, Homo sapiens
gtgaaccccacccctagcagctggagcctggacagtgggaaggaagccaagaacatgtcggatagtgggaaacttatctctccccctgtccctccaagacccacacagactgct

SEQ ID NO: 32 - protein, 38 aa, Homo sapiens
VNPTPSSWSLDSGKEAKNMSDSGKLISPPVPPRPTQTA